(19) United States
(12) Patent Application Publication
(10) Pub. No.: US 2004/0209960 A1
(43) Pub. Date: Oct. 21, 2004

(54) Methods of treating lower urinary tract disorders using sodium channell modulators

(75) Inventors: Edward C. Burgard, Chapel Hill, NC (US); Karl Bruce Thor, Morrisville, NC (US); Matthew Oliver Fraser, Apex, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Dynogen Pharmaceuticals, Inc., Boston, MA

(21) Appl. No.: 10/769,072

(22) Filed: Jan. 30, 2004

Related U.S. Application Data
(60) Provisional application No. 60/443,632, filed on Jan. 30, 2003.
(60) Provisional application No. 60/443,709, filed on Jan. 30, 2003.
(60) Provisional application No. 60/480,321, filed on Jun. 20, 2003.
(60) Provisional application No. 60/480,597, filed on Jun. 20, 2003.
(60) Provisional application No. 60/496,005, filed on Aug. 18, 2003.

(51) Int. Cl. A61K 31/137

(52) U.S. Cl. 514/649

(57) ABSTRACT

The invention relates to methods of using sodium channel modulators, particularly TTX-R sodium channel modulators and/or activity dependent sodium channel modulators to treat painful and non-painful lower urinary tract disorders, particularly painful and non-painful overactive bladder with and/or without loss of urine.

### CROSS-REFERENCE TO RELATED APPLICATIONS

This application claims the benefit of U.S. Provisional Application No. 60/443,632, filed Jan. 30, 2003; U.S. Provisional Application No. 60/443,709, filed Jan. 30, 2003; U.S. Provisional Application No. 60/480,321, filed Jun. 20, 2003; U.S. Provisional Application No. 60/480,597, filed Jun. 20, 2003; and U.S. Provisional Application No. 60/496,005, filed Aug. 18, 2003; all of which are hereby incorporated by reference.

### FIELD OF THE INVENTION

The invention relates to methods of using sodium channel modulators, particularly TTX-R sodium channel modulators and/or activity dependent sodium channel modulators, to treat painful and non-painful lower urinary tract disorders, particularly painful and non-painful overactive bladder.

### BACKGROUND OF THE INVENTION

Lower urinary tract disorders affect the quality of life of millions of men and women in the United States every year. Disorders of the lower urinary tract include overactive bladder, prostatitis and prostadynia, interstitial cystitis, benign prostatic hyperplasia, and, in spinal cord injured patients, and, in spinal cord injured patients, spastic bladder.

Overactive bladder is a treatable medical condition that is estimated to affect 17 to 20 million people in the United States. Current treatments for overactive bladder include medication, diet modification, programs in bladder training, electrical stimulation, and surgery. Currently, antimuscarinics (which are subtypes of the general class of anticholinergics) are the primary medication used for the treatment of overactive bladder. This treatment suffers from limited efficacy and side effects such as dry mouth, dry eyes, dry vagina, palpitations, drowsiness, and constipation, which have proven difficult for some individuals to tolerate.

In recent years, it has been recognized among those of skill in the art that OAB can be divided into urgency without any demonstrable loss of urine as well as urgency with loss of urine. For example, a recent study examined the impact of all OAB symptoms on the quality of life of a community-based sample of the United States population. (Liberman et al. (2001) Urology 57: 1044-1050). This study demonstrated that the group of individuals suffering from OAB without any demonstrable loss of urine have an impaired quality of life when compared with controls. Additionally, individuals with urgency alone have an impaired quality of life compared with controls.

Prostatitis and prostadynia are other lower urinary tract disorders that have been suggested to affect approximately 2-9% of the adult male population (Collins M M, et al., (1998) J. Urology, 159: 1224-1228). Currently, there are no established treatments for prostatitis and prostadynia. Antibiotics are often prescribed, but with little evidence of efficacy. COX-2 selective inhibitors and α-adrenergic blockers and have been suggested as treatments, but their efficacy has not been established. Hot sitz baths and anticholinergic drugs have also been employed to provide some symptomatic relief.

Interstitial cystitis is another lower urinary tract disorder of unknown etiology that predominantly affects young and middle-aged females, although men and children can also be affected. Past treatments for interstitial cystitis have included the administration of antihistamines, sodium pentosanpolysulfate, dimethylsulfoxide, steroids, tricyclic antidepressants and narcotic antagonists, although these methods have generally been unsuccessful (Sant, G. R. (1989) Interstitial cystitis: pathophysiology, clinical evaluation and treatment. Urology Annal 3: 171-196).

Benign prostatic hyperplasia (BPH) is a non-malignant enlargement of the prostate that is very common in men over 40 years of age. Invasive treatments for BPH include transurethral resection of the prostate, transurethral incision of the prostate, balloon dilation of the prostate, prostatic stents, microwave therapy, laser prostatectomy, transrectal high-intensity focused ultrasound therapy and transurethral needle ablation of the prostate. However, complications may arise through the use of some of these treatments, including retrograde ejaculation, impotence, postoperative urinary tract infection and some urinary incontinence. Non-invasive treatments for BPH include androgen deprivation therapy and the use of 5α-reductase inhibitors and α-adrenergic blockers. However, these treatments have proven only minimally to moderately effective for some patients.

Lower urinary tract disorders are particularly problematic for individuals suffering from spinal cord injury. Following spinal cord injury, the bladder is usually affected in one of two ways: 1) “spastic” or “reflex” bladder, in which the bladder fills with urine and a reflex automatically triggers the bladder to empty; or 2) “flaccid” or “non-reflex” bladder, in which the reflexes of the bladder muscles are absent or slowed. Treatment options for these disorders usually include intermittent catheterization, indwelling catheterization, or condom catheterization, but these methods are invasive and frequently inconvenient. Urinary sphincter muscles may also be affected by spinal cord injuries, resulting in an inability of urinary sphincter muscles to relax when the bladder contracts (“dyssynergia”). Traditional treatments for dyssynergia include medications that have been somewhat inconsistent in their efficacy or surgery.

Because existing therapies and treatments for lower urinary tract disorders are associated with limitations as described above, new therapies and treatments are therefore desirable.

### SUMMARY OF THE INVENTION

Compositions and methods for treating painful and non-painful lower urinary tract disorders, particularly painful and non-painful overactive bladder with and/or without loss of urine, are provided. Compositions of the invention comprise sodium channel modulators, particularly tetrodotoxin-resistant (TTX-R) sodium channel modulators and/or activity-dependent sodium channel modulators as well as pharmaceutically acceptable, pharmacologically active salts, enantiomers, analogs, esters, amides, prodrugs, metabolites, and derivatives. TTX-R sodium channel modulators for use in the present invention include but are not limited to compounds that modulate or interact with Nav.1.8 and/or Naᵥ1.9 channels.

The compositions are administered in therapeutically effective amounts to a patient in need thereof for treating painful and non-painful lower urinary tract disorders, in mammals, particularly humans. It is recognized that the compositions may be administered by any means of administration as long as an effective amount for the treatment of painful and non-painful symptoms associated with lower urinary tract disorders is delivered. The compositions may be formulated, for example, for sustained, continuous, or as-needed administration.

### BRIEF DESCRIPTION OF THE DRAWINGS

FIG. 1. FIG. 1 depicts bladder capacity before (Sal) and after (remaining groups) bladder hyperactivity caused by continuous intravesical dilute acetic acid infusion. Ambroxol was administered intraduodenally at increasing doses. Note that Ambroxol was capable of partially reversing the reduction in bladder capacity caused by acetic acid in a dose-dependent fashion. Responses from each individual were normalized to their respective post-irritation vehicle control values and the data are expressed as Mean±SEM.

FIG. 2. FIG. 2 depicts bladder capacity before (Sal) and after (remaining groups) bladder hyperactivity caused by continuous intravesical dilute acetic acid infusion. Ralfinamide was administered intraduodenally at increasing doses. Note that Ralfinamide was capable of partially reversing the reduction in bladder capacity caused by acetic acid in a dose-dependent fashion. Responses from each individual were normalized to their respective post-irritation vehicle control values and the data are expressed as Mean±SEM.

FIG. 3. FIG. 3 depicts bladder capacity before (Sal) and after (remaining groups) bladder hyperactivity caused by continuous intravesical dilute acetic acid infusion. Carbamazepine was administered intraduodenally at increasing doses. Note that Carbamazepine was capable of partially reversing the reduction in bladder capacity caused by acetic acid in a dose-dependent fashion. Responses from each individual were normalized to their respective post-irritation vehicle control values and the data are expressed as Mean±SEM.

FIG. 4. FIG. 4 depicts bladder capacity before (Sal) and after (remaining groups) bladder hyperactivity caused by continuous intravesical dilute acetic acid infusion. Topiramate was administered intraduodenally at increasing doses. Note that Topiramate was capable of partially reversing the reduction in bladder capacity caused by acetic acid in a dose-dependent fashion. Responses from each individual were normalized to their respective post-irritation vehicle control values and the data are expressed as Mean±SEM.

FIG. 5. FIG. 5 depicts bladder capacity before (Sal) and after (remaining groups) bladder hyperactivity caused by continuous intravesical dilute acetic acid infusion. Sipatrigine was administered intraduodenally at increasing doses. Note that Sipatrigine was capable of partially reversing the reduction in bladder capacity caused by acetic acid in a dose-dependent fashion. Responses from each individual were normalized to their respective post-irritation vehicle control values and the data are expressed as Mean±SEM.

FIG. 6. FIG. 6 depicts bladder capacity before (Sal) and after (remaining groups) bladder hyperactivity caused by continuous intravesical dilute acetic acid infusion. Losigamone was administered intraduodenally at increasing doses. Note that Losigamone was capable of partially reversing the reduction in bladder capacity caused by acetic acid in a dose-dependent fashion. Responses from each individual were normalized to their respective post-irritation vehicle control values and the data are expressed as Mean±SEM.

FIG. 7. FIG. 7 depicts bladder capacity before (Sal) and after (remaining groups) bladder hyperactivity caused by continuous intravesical dilute acetic acid infusion. Mexiletine was administered intraduodenally at increasing doses. Note that Mexiletine was capable of partially reversing the reduction in bladder capacity caused by acetic acid in a dose-dependent fashion. Responses from each individual were normalized to their respective post-irritation vehicle control values and the data are expressed as Mean±SEM.

FIG. 8. FIG. 8 depicts bladder capacity before (Sal) and after (remaining groups) bladder hyperactivity caused by continuous intravesical dilute acetic acid infusion. Lidocaine was administered intravenously at increasing doses. Note that Lidocaine was capable of partially reversing the reduction in bladder capacity caused by acetic acid in a dose-dependent fashion. Responses from each individual were normalized to their respective post-irritation vehicle control values and the data are expressed as Mean±SEM.

FIG. 9. FIG. 9 depicts bladder capacity before (Sal) and after (remaining groups) bladder hyperactivity caused by continuous intravesical dilute acetic acid infusion. Vinpocetine was administered intraduodenally at increasing doses. Note that Vinpocetine was not capable of significantly reversing the reduction in bladder capacity caused by acetic acid. Responses from each individual were normalized to their respective post-irritation vehicle control values and the data are expressed as Mean±SEM.

FIG. 10. FIG. 10 depicts bladder capacity before (Sal) and after (remaining groups) bladder hyperactivity caused by continuous intravesical dilute acetic acid infusion. Tolperisone was administered intravenously at increasing doses. Note that Tolperisone was not capable of significantly reversing the reduction in bladder capacity caused by acetic acid. Responses from each individual were normalized to their respective post-irritation vehicle control values and the data are expressed as Mean±SEM.

FIG. 11. FIG. 11A depicts representative TTX-R sodium currents recorded from a labeled bladder afferent neuron before and during bath application of Ambroxol. FIG. 11B depicts a reversible, concentration-dependent reduction in current amplitude following 2-3 minute application of Ambroxol.

FIG. 12. FIG. 12 depicts a typical inward TTX-R sodium current recorded from a labeled bladder afferent neuron before and during bath application of ralfinamide.

FIG. 13. FIG. 13 depicts a typical inward TTX-R sodium current recorded from a labeled bladder afferent neuron before and during bath application of topiramate.

FIG. 14. FIG. 14A depicts a typical inward TTX-R sodium current recorded from a labeled bladder afferent neuron before and during bath application of sipatrigine. FIG. 14B depicts a summary bar chart showing the combined effects of sipatrigine on 2-5 separate bladder afferent neurons.

FIG. 15. FIG. 15A depicts a typical response to lamotrigine under both slow and fast stimulation of sodium currents. FIG. 15B depicts summary data obtained from three neurons under control conditions and during application of 100 μM lamotrigine.

### DETAILED DESCRIPTION OF THE INVENTION

Overview and Definitions

The present invention provides compositions and methods for treating painful and non-painful lower urinary tract disorders, including such disorders as overactive bladder with and/or without loss of urine, urinary frequency, urinary urgency, and nocturia. The compositions comprise a therapeutically effective dose of sodium channel modulators, particularly tetrodotoxin-resistant (TTX-R) sodium channel modulators and/or activity-dependent sodium channel modulators. The methods are accomplished by administering, for example, various compositions and formulations that contain quantities of a sodium channel modulator, particularly a tetrodotoxin-resistant (TTX-R) sodium channel modulator and/or activity-dependent sodium channel modulator.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

It must be noted that as used in this specification and the appended embodiments, the singular forms “a,” an” and “the” include plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

By “non-painful” is intended sensations or symptoms including mild or general discomfort that a patient subjectively describes as not producing or resulting in pain.

By “painful” is intended sensations or symptoms that a patient subjectively describes as producing or resulting in pain.

By “lower urinary tract” is intended all parts of the urinary system except the kidneys. By “lower urinary tract disorder” is intended any disorder involving the lower urinary tract, including but not limited to overactive bladder, prostatitis, interstitial cystitis, benign prostatic hyperplasia, and, in spinal cord injured patients, spastic bladder. By “non-painful lower urinary tract disorder” is intended any lower urinary tract disorder involving sensations or symptoms, including mild or general discomfort, that a patient subjectively describes as not producing or resulting in pain. By “painful lower urinary tract disorder” is intended any lower urinary tract disorder involving sensations or symptoms that a patient subjectively describes as producing or resulting in pain.

By “bladder disorder” is intended any condition involving the urinary bladder. By “non-painful bladder disorder” is intended any bladder disorder involving sensations or symptoms, including mild or general discomfort, that a patient subjectively describes as not producing or resulting in pain. By “painful bladder disorder” is intended any bladder disorder involving sensations or symptoms that a patient subjectively describes as producing or resulting in pain.

By “overactive bladder” is intended any form of lower urinary tract disorder characterized by increased frequency of micturition or the desire to void, whether complete or episodic, and where loss of voluntary control ranges from partial to total and whether there is loss of urine (incontinence) or not. By “painful overactive bladder” is intended any form of overactive bladder, as defined above, involving sensations or symptoms that a patient subjectively describes as producing or resulting in pain. By “non-painful overactive bladder” is intended any form of overactive bladder, as defined above, involving sensations or symptoms, including mild or general discomfort, that a patient subjectively describes as not producing or resulting in pain. Non-painful symptoms can include, but are not limited to, urinary urgency, incontinence, urge incontinence, stress incontinence, urinary frequency, and nocturia.

“OAB wet” is used herein to describe overactive bladder in patients with incontinence, while “OAB dry” is used herein to describe overactive bladder in patients without incontinence.

By “urinary urgency” is intended sudden strong urges to urinate with little or no chance to postpone the urination. By “incontinence” is meant the inability to control excretory functions, including urination (urinary incontinence). By “urge incontinence” or “urinary urge incontinence” is intended the involuntary loss of urine associated with an abrupt and strong desire to void. By “stress incontinence” or “urinary stress incontinence” is intended a medical condition in which urine leaks when a person coughs, sneezes, laughs, exercises, lifts heavy objects, or does anything that puts pressure on the bladder. By “urinary frequency” is intended urinating more frequently than the patient desires. As there is considerable interpersonal variation in the number of times in a day that an individual would normally expect to urinate, “more frequently than the patient desires” is further defined as a greater number of times per day than that patient's historical baseline. “Historical baseline” is further defined as the median number of times the patient urinated per day during a normal or desirable time period. By “nocturia” is intended being awakened from sleep to urinate more frequently than the patient desires.

By “neurogenic bladder” or “neurogenic overactive bladder” is intended overactive bladder as described further herein that occurs as the result of neurological damage due to disorders including but not limited to stroke, Parkinson's disease, diabetes, multiple sclerosis, peripheral neuropathy, or spinal cord lesions.

By “detrusor hyperreflexia” is intended a condition characterized by uninhibited detrusor, wherein the patient has some sort of neurologic impairment. By “detrusor instability” or “unstable detrusor” is intended conditions where there is no neurologic abnormality.

By “prostatitis” is intended any type of disorder associated with an inflammation of the prostate, including chronic bacterial prostatitis and chronic non-bacterial prostatitis. By “non-painful prostatitis” is intended prostatitis involving sensations or symptoms, including mild or general discomfort, that a patient subjectively describes as not producing or resulting in pain. By “painful prostatitis” is intended prostatitis involving sensations or symptoms that a patient subjectively describes as producing or resulting in pain.

“Chronic bacterial prostatitis” is used in its conventional sense to refer to a disorder associated with symptoms that include inflammation of the prostate and positive bacterial cultures of urine and prostatic secretions. “Chronic non-bacterial prostatitis” is used in its conventional sense to refer to a disorder associated with symptoms that include inflammation of the prostate and negative bacterial cultures of urine and prostatic secretions. “Prostadynia” is used in its conventional sense to refer to a disorder generally associated with painful symptoms of chronic non-bacterial prostatitis as defined above, without inflammation of the prostate. “Interstitial cystitis” is used in its conventional sense to refer to a disorder associated with symptoms that include irritative voiding symptoms, urinary frequency, urgency, nocturia, and suprapubic or pelvic pain related to and relieved by voiding.

“Benign prostatic hyperplasia” is used in its conventional sense to refer to a disorder associated with benign enlargement of the prostate gland.

“Spastic bladder” or “reflex bladder” is used in its conventional sense to refer to a condition following spinal cord injury in which bladder emptying has become unpredictable.

“Flaccid bladder” or “non-reflex bladder” is used in its conventional sense to refer to a condition following spinal cord injury in which the reflexes of the bladder muscles are absent or slowed.

“Dyssynergia” is used in its conventional sense to refer to a condition following spinal cord injury in which patients characterized by an inability of urinary sphincter muscles to relax when the bladder contracts.

The terms “active agent” and “pharmacologically active agent” are used interchangeably herein to refer to a chemical compound that induces a desired effect, i.e., in this case, treatment of painful and non-painful lower urinary tract disorders, such as painful and non-painful overactive bladder with and/or without loss of urine. The primary active agents herein are compounds that interact with TTX-R sodium channels, including but not limited to sodium channel modulators, particularly tetrodotoxin-resistant (TTX-R) sodium channel modulators and/or activity-dependent sodium channel modulators, including compounds that modulate or interact with Nav1.8 and/or Naᵥ1.9 channels. In addition, a combination therapy wherein a sodium channel modulator, particularly a tetrodotoxin-resistant (TTX-R) sodium channel modulator and/or activity-dependent sodium channel modulator is administered with one or more additional active agents is also within the scope of the present invention. Such combination therapy may be carried out by administration of the different active agents in a single composition, by concurrent administration of the different active agents in different compositions, or by sequential administration of the different active agents. Included are salts, enantiomers, analogs, esters, amides, prodrugs, active metabolites, and derivatives of those compounds or classes of compounds specifically mentioned that also induce the desired effect.

The term “sodium channel modulator” as used herein is intended to include agents that interact with the channel pore itself (e.g., a binding event), or that may act as an allosteric modulator of the channel by interacting with a site on the channel complex (e.g., a binding event), as well as salts, esters, amides, prodrugs, active metabolites, and other derivatives thereof. Further, it is understood that any salts, enantiomers, analogs, esters, amides, prodrugs, metabolites, or derivatives are pharmaceutically acceptable as well as pharmacologically active.

The term TTX-R sodium channel modulator as used herein is intended to include agents that interact with TTX-R sodium channels and/or any protein associated with a TTX-R sodium channels (e.g., a binding event) to produce a physiological effect, such as opening, closing, blocking, up-regulating expression, or down-regulating expression of the channel, but not antisense or knockout technologies. “Agents that interact with TTX-R sodium channels and/or any protein associated with a TTX-R sodium channel” include but are not limited to, amino acid compounds, peptide, nonpeptide, peptidomimetic, small molecular weight organic compounds, and other compounds that modulate or interact with TTX-R sodium channels (e.g., a binding event) or proteins associates with TTX-R sodium channels (e.g., a binding event) such as anchor proteins, as well as salts, esters, amides, prodrugs, active metabolites, and other derivatives thereof. “Agents that interact with TTX-R sodium channels and/or any protein associated with a TTX-R sodium channel” also include but are not limited to, amino acid compounds, peptide, nonpeptide, peptidomimetic, small molecular weight organic compounds, and other compounds that modulate or interact with Nav.1.8 and/or Na_{v.}1.9 channels (e.g., a binding event) or proteins associated with Nav.1.8 and/or Na_{v.}1.9 channels (e.g., a binding event), such as anchor proteins, as well as salts, esters, amides, prodrugs, active metabolites, and other derivatives thereof. Further, it is understood that any salts, enantiomers, analogs, esters, amides, prodrugs, metabolites, or derivatives are pharmaceutically acceptable as well as pharmacologically active.

The term “activity-dependent sodium channel modulator” or “use-dependent sodium channel modulator” as used herein is intended an agent that preferentially modulates the activity of a sodium channel that has been activated or opened, and exhibits its effect either by modifying the activity of the open channel, or by modifying the activity of the inactivated state of the channel as described in Hille B. (1992) Ionic Channels in Excitable Membranes. 2nd ed. Sinauer Associates, Sunderland, Mass., pp. 390-422. Unless otherwise indicated, the term “activity-dependent sodium channel modulator” is intended to include agents that interact with the channel pore itself (e.g., a binding event), or that may act as an allosteric modulator of the channel by interacting with a site on the channel complex (e.g., a binding event), as well as salts, esters, amides, prodrugs, active metabolites, and other derivatives thereof. Further, it is understood that any salts, enantiomers, analogs, esters, amides, prodrugs, metabolites, or derivatives are pharmaceutically acceptable as well as pharmacologically active.

The term “peptidomimetic” is used in its conventional sense to refer to a molecule that mimics the biological activity of a peptide but is no longer peptidic in chemical nature, including molecules that lack amide bonds between amino acids, as well as pseudo-peptides, semi-peptides and peptoids. Peptidomimetics according to this invention provide a spatial arrangement of reactive chemical moieties that closely resembles the three-dimensional arrangement of active groups in the peptide on which the peptidomimetic is based. As a result of this similar active-site geometry, the peptidomimetic has effects on biological systems that are similar to the biological activity of the peptide.

The term “anticholinergic agent” as used herein refers to any acetylcholine receptor antagonist, including antagonists of nicotinic and/or muscarinic acetylcholine receptors. The term “antinicotinic agent” as used herein is intended any nicotinic acytylcholine receptor antagonist. The term “antimuscarinic agent” as used herein is intended any muscarinic acetylcholine receptor antagonist. Unless otherwise indicated, the terms “anticholinergic agent,” “antinicotinic agent,” and “antimuscarinic agent” are intended to include anticholinergic, antinicotinic, and antimuscarinic agents as disclosed further herein, as well as salts, esters, amides, prodrugs, active metabolites, and other derivatives thereof. Further, it is understood that any salts, enantiomers, analogs, esters, amides, prodrugs, metabolites, or derivatives are pharmaceutically acceptable as well as pharmacologically active.

The term “β3 adrenergic agonist” is used in its conventional sense to refer to a compound that agonizes β3 adrenergic receptors. Unless otherwise indicated, the term “β3 adrenergic agonist” is intended to include β3 adrenergic agonist agents as disclosed further herein, as well as salts, enantiomers, analogs, esters, amides, prodrugs, metabolites, or derivatives thereof. Further, it is understood that any salts, enantiomers, analogs, esters, amides, prodrugs, metabolites, or derivatives are pharmaceutically acceptable as well as pharmacologically active.

The term “spasmolytic” (also known as “antispasmodic”) is used in its conventional sense to refer to a compound that relieves or prevents muscle spasms, especially of smooth muscle. Unless otherwise indicated, the term “spasmolytic” is intended to include spasmolytic agents as disclosed further herein, as well as salts, enantiomers, analogs, esters, amides, prodrugs, metabolites, or derivatives thereof. Further, it is understood that any salts, enantiomers, analogs, esters, amides, prodrugs, metabolites, or derivatives are pharmaceutically acceptable as well as pharmacologically active.

The term “neurokinin receptor antagonist” is used in its conventional sense to refer to a compound that antagonizes neurokinin receptors. Unless otherwise indicated, the term “neurokinin receptor antagonist” is intended to include neurokinin receptor antagonist agents as disclosed further herein, as well as salts, esters, amides, prodrugs, active metabolites, and other derivatives thereof. Further, it is understood that any salts, enantiomers, analogs, esters, amides, prodrugs, metabolites, or derivatives are pharmaceutically acceptable as well as pharmacologically active.

The term “bradykinin receptor antagonist” is used in its conventional sense to refer to a compound that antagonizes bradykinin receptors. Unless otherwise indicated, the term “bradykinin receptor antagonist” is intended to include bradykinin receptor antagonist agents as disclosed further herein, as well as salts, esters, amides, prodrugs, active metabolites, and other derivatives thereof. Further, it is understood that any salts, enantiomers, analogs, esters, amides, prodrugs, metabolites, or derivatives are pharmaceutically acceptable as well as pharmacologically active.

The term “nitric oxide donor” is used in its conventional sense to refer to a compound that releases free nitric oxide when administered to a patient. Unless otherwise indicated, the term “nitric oxide donor” is intended to include nitric oxide donor agents as disclosed further herein, as well as salts, esters, amides, prodrugs, active metabolites, and other derivatives thereof. Further, it is understood that any salts, enantiomers, analogs, esters, amides, prodrugs, metabolites, or derivatives are pharmaceutically acceptable as well as pharmacologically active.

The terms “treating” and “treatment” as used herein refer to relieving the painful or non-painful symptoms or lessening the discomfort associated with lower urinary tract disorders, particularly painful or non-painful overactive bladder as well as overactive bladder with and/or without loss of urine, in mammals, particularly humans.

By an “effective” amount or a “therapeutically effective amount” of a drug or pharmacologically active agent is meant a nontoxic but sufficient amount of the drug or agent to provide the desired effect, i.e., relieving the painful and non-painful symptoms or lessening the discomfort associated with lower urinary tract disorders, particularly painful and non-painful overactive bladder, as explained above.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative or metabolite, refers to a derivative or metabolite having the same type of pharmacological activity as the parent compound. When the term “pharmaceutically acceptable” is used to refer to a derivative (e.g., a salt or an analog) of an active agent, it is to be understood that the compound is pharmacologically active as well, i.e., therapeutically effective for treating painful and non-painful lower urinary tract disorders, such as overactive bladder with and/or without loss of urine, in mammals, particularly humans.

By “continuous” dosing is meant the chronic administration of a selected active agent.

By “as-needed” dosing, also known as “pro re nata” “prn” dosing, and “on demand” dosing or administration is meant the administration of a single dose of the active agent at some time prior to commencement of an activity wherein suppression of the painful and non-painful symptoms of a lower urinary tract disorder, such as overactive bladder with and/or without loss of urine, would be desirable. Administration can be immediately prior to such an activity, including about 0 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, or about 10 hours prior to such an activity, depending on the formulation.

By “short-term” is intended any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

By “rapid-offset” is intended any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

The term “controlled release” is intended to refer to any drug-containing formulation in which release of the drug is not immediate, i.e., with a “controlled release” formulation, oral administration does not result in immediate release of the drug into an absorption pool. The term is used interchangeably with “non-immediate release” as defined in Remington: The Science and Practice of Pharmacy, Twentieth Ed. (Philadelphia, Pa.: Lippincott Williams & Wilkins, 2000).

The “absorption pool” represents a solution of the drug administered at a particular absorption site, and kᵣ, kₐ, and kₑ are first-order rate constants for: 1) release of the drug from the formulation; 2) absorption; and 3) elimination, respectively. For immediate release dosage forms, the rate constant for drug release kᵣ is far greater than the absorption rate constant kₐ. For controlled release formulations, the opposite is true, i.e., kᵣ<<<kₐ, such that the rate of release of drug from the dosage form is the rate-limiting step in the delivery of the drug to the target area. The term “controlled release” as used herein includes any nonimmediate release formulation, including but not limited to sustained release, delayed release and pulsatile release formulations.

The term “sustained release” is used in its conventional sense to refer to a drug formulation that provides for gradual release of a drug over an extended period of time, and that preferably, although not necessarily, results in substantially constant blood levels of a drug over an extended time period such as up to about 72 hours, about 66 hours, about 60 hours, about 54 hours, about 48 hours, about 42 hours, about 36 hours, about 30 hours, about 24 hours, about 18 hours, about 12 hours, about 10 hours, about 8 hours, about 7 hours, about 6 hours, about 5 hours, about 4 hours, about 3 hours, about 2 hours, or about 1 hour after drug administration.

The term “delayed release” is used in its conventional sense to refer to a drug formulation that provides for an initial release of the drug after some delay following drug administration and that preferably, although not necessarily, includes a delay of up to about 10 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, or about 12 hours.

The term “pulsatile release” is used in its conventional sense to refer to a drug formulation that provides release of the drug in such a way as to produce pulsed plasma profiles of the drug after drug administration. The term “immediate release” is used in its conventional sense to refer to a drug formulation that provides for release of the drug immediately after drug administration.

The term “immediate release” is used in its conventional sense to refer to a drug formulation that provides for release of the drug immediately after drug administration.

By the term “transdermal” drug delivery is meant delivery by passage of a drug through the skin or mucosal tissue and into the bloodstream.

The term “topical administration” is used in its conventional sense to mean delivery of a topical drug or pharmacologically active agent to the skin or mucosa.

The term “oral administration” is used in its conventional sense to mean delivery of a drug through the mouth and ingestion through the stomach and digestive tract.

The term “inhalation administration” is used in its conventional sense to mean delivery of an aerosolized form of the drug by passage through the nose or mouth during inhalation and passage of the drug through the walls of the lungs.

The term “intravesical administration” is used in its conventional sense to mean delivery of a drug directly into the bladder.

By the term “parenteral” drug delivery is meant delivery by passage of a drug into the blood stream without first having to pass through the alimentary canal, or digestive tract. Parenteral drug delivery may be “subcutaneous,” referring to delivery of a drug by administration under the skin. Another form of parenteral drug delivery is “intramuscular,” referring to delivery of a drug by administration into muscle tissue. Another form of parenteral drug delivery is “intradermal,” referring to delivery of a drug by administration into the skin. An additional form of parenteral drug delivery is “intravenous,” referring to delivery of a drug by administration into a vein. An additional form of parenteral drug delivery is “intra-arterial,” referring to delivery of a drug by administration into an artery. Another form of parenteral drug delivery is “transdermal,” referring to delivery of a drug by passage of the drug through the skin and into the bloodstream.

Still another form of parenteral drug delivery is “transmucosal,” referring to administration of a drug to the mucosal surface of an individual so that the drug passes through the mucosal tissue and into the individual's blood stream. Transmucosal drug delivery may be “buccal” or “transbuccal,” referring to delivery of a drug by passage through an individual's buccal mucosa and into the bloodstream. Another form of transmucosal drug delivery herein is “lingual” drug delivery, which refers to delivery of a drug by passage of a drug through an individual's lingual mucosa and into the bloodstream. Another form of transmucosal drug delivery herein is “sublingual” drug delivery, which refers to delivery of a drug by passage of a drug through an individual's sublingual mucosa and into the bloodstream. Another form of transmucosal drug delivery is “nasal” or “intranasal” drug delivery, referring to delivery of a drug through an individual's nasal mucosa and into the bloodstream. An additional form of transmucosal drug delivery herein is “rectal” or “transrectal” drug delivery, referring to delivery of a drug by passage of a drug through an individual's rectal mucosa and into the bloodstream. Another form of transmucosal drug delivery is “urethral” or “transurethral” delivery, referring to delivery of the drug into the urethra such that the drug contacts and passes through the wall of the urethra. An additional form of transmucosal drug delivery is “vaginal” or “transvaginal” delivery, referring to delivery of a drug by passage of a drug through an individual's vaginal mucosa and into the bloodstream. An additional form of transmucosal drug delivery is “perivaginal” delivery, referring to delivery of a drug through the vaginolabial tissue into the bloodstream.

In order to carry out the method of the invention, a selected active agent is administered to a patient suffering from a painful or non-painful lower urinary tract disorder, such as painful or non-painful overactive bladder as well as overactive bladder with and/or without loss of urine. A therapeutically effective amount of the active agent may be administered orally, intravenously, subcutaneously, transmucosally (including buccally, sublingually, transurethrally, and rectally), topically, transdermally, by inhalation, intravesically or using any other route of administration.

Lower Urinary Tract Disorders

Lower urinary tract disorders affect the quality of life of millions of men and women in the United States every year. While the kidneys filter blood and produce urine, the lower urinary tract is concerned with storage and elimination of this waste liquid and includes all other parts of the urinary tract except the kidneys. Generally, the lower urinary tract includes the ureters, the urinary bladder, and the urethra. Disorders of the lower urinary tract include painful and non-painful overactive bladder, prostatitis and prostadynia, interstitial cystitis, benign prostatic hyperplasia, and, in spinal cord injured patients, spastic bladder.

Overactive bladder is a treatable medical condition that is estimated to affect 17 to 20 million people in the United States. Symptoms of overactive bladder include urinary frequency, urgency, nocturia (the disturbance of nighttime sleep because of the need to urinate) and urge incontinence (accidental loss of urine) due to a sudden and unstoppable need to urinate. As opposed to stress incontinence, in which loss of urine is associated with physical actions such as coughing, sneezing, exercising, or the like, urge incontinence is usually associated with an overactive detrusor muscle (the smooth muscle of the bladder which contracts and causes it to empty).

There is no single etiology for overactive bladder. Neurogenic overactive bladder (or neurogenic bladder) occurs as the result of neurological damage due to disorders such as stroke, Parkinson's disease, diabetes, multiple sclerosis, peripheral neuropathy, or spinal cord lesions. In these cases, the overactivity of the detrusor muscle is termed detrusor hyperreflexia. By contrast, non-neurogenic overactive bladder can result from non-neurological abnormalities including bladder stones, muscle disease, urinary tract infection or drug side effects.

Due to the enormous complexity of micturition (the act of urination) the exact mechanism causing overactive bladder is unknown. Overactive bladder may result from hypersensitivity of sensory neurons of the urinary bladder, arising from various factors including inflammatory conditions, hormonal imbalances, and prostate hypertrophy. Destruction of the sensory nerve fibers, either from a crushing injury to the sacral region of the spinal cord, or from a disease that causes damage to the dorsal root fibers as they enter the spinal cord may also lead to overactive bladder. In addition, damage to the spinal cord or brain stem causing interruption of transmitted signals may lead to abnormalities in micturition. Therefore, both peripheral and central mechanisms may be involved in mediating the altered activity in overactive bladder.

In spite of the uncertainty regarding whether central or peripheral mechanisms, or both, are involved in overactive bladder, many proposed mechanisms implicate neurons and pathways that mediate non-painful visceral sensation. Pain is the perception of an aversive or unpleasant sensation and may arise through a variety of proposed mechanisms. These mechanisms include activation of specialized sensory receptors that provide information about tissue damage (nociceptive pain), or through nerve damage from diseases such as diabetes, trauma or toxic doses of drugs (neuropathic pain) (See, e.g., A. I. Basbaum and T. M. Jessell (2000) The perception of pain. In Principles of Neural Science, 4th. ed.; Benevento et al. (2002) Physical Therapy Journal 82:601-12). Nociception may give rise to pain, but not all stimuli that activate nociceptors are experienced as pain (A. I. Basbaum and T. M. Jessell (2000) The perception of pain. In Principles of Neural Science, 4th. ed.). Somatosensory information from the bladder is relayed by nociceptive Aδ and C fibers that enter the spinal cord via the dorsal root ganglion (DRG) and project to the brainstem and thalamus via second or third order neurons (Andersson (2002) Urology 59:18-24; Andersson (2002) Urology 59:43-50; Morrison, J., Steers, W. D., Brading, A., Blok, B., Fry, C., de Groat, W. C., Kakizaki, H., Levin, R., and Thor, K. B., “Basic Urological Sciences” In: Incontinence (vol. 2) Abrams, P. Khoury, S., and Wein, A. (Eds.) Health Publications, Ltd., Plymbridge Distributors, Ltd., Plymouth, UK., (2002)). A number of different subtypes of sensory afferent neurons may be involved in neurotransmission from the lower urinary tract. These may be classified as, but not limited to, small diameter, medium diameter, large diameter, myelinated, unmyelinated, sacral, lumbar, peptidergic, non-peptidergic, IB4 positive, IB4 negative, C fiber, Aδ fiber, high threshold or low threshold neurons. Nociceptive input to the DRG is thought to be conveyed to the brain along several ascending pathways, including the spinothalamic, spinoreticular, spinomesencephalic, spinocervical, and in some cases dorsal column/medial lemniscal tracts (A. I. Basbaum and T. M. Jessell (2000) The perception of pain. In Principles of Neural Science, 4th. ed.). Central mechanisms, which are not fully understood, are thought to convert some, but not all, nociceptive information into painful sensory perception (A. I. Basbaum and T. M. Jessell (2000) The perception of pain. In Principles of Neural Science, 4th. ed.). Although many compounds have been explored as treatments for disorders involving pain of the bladder or other pelvic visceral organs, relatively little work has been directed toward treatment of non-painful sensory symptoms associated with bladder disorders such as overactive bladder.

The compounds of the present invention are useful in the treatment of both painful and non-painful overactive bladder. Current treatments for overactive bladder include medication, diet modification, programs in bladder training, electrical stimulation, and surgery. Currently, antimuscarinics (which are subtypes of the general class of anticholinergics) are the primary medication used for the treatment of overactive bladder. This treatment suffers from limited efficacy and side effects such as dry mouth, dry eyes, dry vagina, palpitations, drowsiness, and constipation, which have proven difficult for some individuals to tolerate. Therefore, the compounds of the present invention meet an existing need for new treatments for both painful and non-painful overactive bladder.

Overactive bladder (or OAB) can occur with or without incontinence. In recent years, it has been recognized among those of skill in the art that the cardinal symptom of OAB is urgency without regard to any demonstrable loss of urine. For example, a recent study examined the impact of all OAB symptoms on the quality of life of a community-based sample of the United States population. (Liberman et al. (2001) Urology 57: 1044-1050). This study demonstrated that individuals suffering from OAB without any demonstrable loss of urine have an impaired quality of life when compared with controls. Additionally, individuals with urgency alone have an impaired quality of life compared with controls.

Although urgency is now believed to be the primary symptom of OAB, to date it has not been evaluated in a quantified way in clinical studies. Corresponding to this new understanding of OAB, however, the terms OAB Wet (with incontinence) and OAB Dry (without incontinence) have been proposed to describe these different patient populations (see, e.g., WO03/051354). The prevalence of OAB Wet and OAB Dry is reported to be similar in men and women, with a prevalence rate in the United States of 16.6% (Stewart et al., “Prevalence of Overactive Bladder in the United States: Results from the NOBLE Program,” Abstract Presented at the Second International Consultation on Incontinence, July 2001, Paris, France). In particular, the compounds of the present invention are useful in the treatment of OAB Wet and OAB Dry.

Prostatitis and prostadynia are other lower urinary tract disorders that have been suggested to affect approximately 2-9% of the adult male population (Collins M M, et al., (1998) “How common is prostatitis? A national survey of physician visits,” Journal of Urology, 159: 1224-1228). Prostatitis is associated with an inflammation of the prostate, and may be subdivided into chronic bacterial prostatitis and chronic non-bacterial prostatitis. Chronic bacterial prostatitis is thought to arise from bacterial infection and is generally associated with such symptoms as inflammation of the prostate, the presence of white blood cells in prostatic fluid, and/or pain. Chronic non-bacterial prostatitis is an inflammatory and painful condition of unknown etiology characterized by excessive inflammatory cells in prostatic secretions despite a lack of documented urinary tract infections, and negative bacterial cultures of urine and prostatic secretions. Prostadynia (chronic pelvic pain syndrome) is a condition associated with the painful symptoms of chronic non-bacterial prostatitis without an inflammation of the prostate.

The compounds of the present invention are useful for the treatment of prostatitis and prostadynia. Currently, there are no established treatments for prostatitis and prostadynia. Antibiotics are often prescribed, but with little evidence of efficacy. COX-2 selective inhibitors and α-adrenergic blockers and have been suggested as treatments, but their efficacy has not been established. Hot sitz baths and anticholinergic drugs have also been employed to provide some symptomatic relief. Therefore, the compounds of the present invention meet an existing need for new treatments for prostatitis and prostadynia.

Interstitial cystitis is another lower urinary tract disorder of unknown etiology that predominantly affects young and middle-aged females, although men and children can also be affected. Symptoms of interstitial cystitis may include irritative voiding symptoms, urinary frequency, urgency, nocturia and suprapubic or pelvic pain related to and relieved by voiding. Many interstitial cystitis patients also experience headaches as well as gastrointestinal and skin problems. In some extreme cases, interstitial cystitis may also be associated with ulcers or scars of the bladder.

The compounds of the present invention are useful for the treatment of interstitial cystitis. Past treatments for interstitial cystitis have included the administration of antihistamines, sodium pentosanpolysulfate, dimethylsulfoxide, steroids, tricyclic antidepressants and narcotic antagonists, although these methods have generally been unsuccessful (Sant, G. R. (1989) Interstitial cystitis: pathophysiology, clinical evaluation and treatment. Urology Annal 3: 171-196). Therefore, the compounds of the present invention meet an existing need for new treatments for interstitial cystitis.

Benign prostatic hyperplasia (BPH) is a non-malignant enlargement of the prostate that is very common in men over 40 years of age. BPH is thought to be due to excessive cellular growth of both glandular and stromal elements of the prostate. Symptoms of BPH include urinary frequency, urge incontinence, nocturia, and reduced urinary force and speed of flow.

The compounds of the present invention are useful for the treatment of BPH. Invasive treatments for BPH include transurethral resection of the prostate, transurethral incision of the prostate, balloon dilation of the prostate, prostatic stents, microwave therapy, laser prostatectomy, transrectal high-intensity focused ultrasound therapy and transurethral needle ablation of the prostate. However, complications may arise through the use of some of these treatments, including retrograde ejaculation, impotence, postoperative urinary tract infection and some urinary incontinence. Non-invasive treatments for BPH include androgen deprivation therapy and the use of 5α-reductase inhibitors and α-adrenergic blockers. However, these treatments have proven only minimally to moderately effective for some patients. Therefore, the compounds of the present invention meet an existing need for new treatments for BPH.

The compounds of the present invention are also useful for treating lower urinary tract disorders in spinal cord injured patients. After spinal cord injury, the kidneys continue to make urine, and urine can continue to flow through the ureters and urethra because they are the subject of involuntary neural and muscular control, with the exception of conditions where bladder to smooth muscle dyssenergia is present. By contrast, bladder and sphincter muscles are also subject to voluntary neural and muscular control, meaning that descending input from the brain through the spinal cord drives bladder and sphincter muscles to completely empty the bladder. Following spinal cord injury, such descending input may be disrupted such that individuals may no longer have voluntary control of their bladder and sphincter muscles. Spinal cord injuries can also disrupt sensory signals that ascend to the brain, preventing such individuals from being able to feel the urge to urinate when their bladder is full.

Following spinal cord injury, the bladder is usually affected in one of two ways. The first is a condition called “spastic” or “reflex” bladder, in which the bladder fills with urine and a reflex automatically triggers the bladder to empty. This usually occurs when the injury is above the T12 level. Individuals with spastic bladder are unable to determine when, or if, the bladder will empty. The second is “flaccid” or “non-reflex” bladder, in which the reflexes of the bladder muscles are absent or slowed. This usually occurs when the injury is below the T12/L1 level. Individuals with flaccid bladder may experience over-distended or stretched bladders and “reflux” of urine through the ureters into the kidneys. Treatment options for these disorders usually include intermittent catheterization, indwelling catheterization, or condom catheterization, but these methods are invasive and frequently inconvenient. Therefore, the compounds of the present invention meet an existing need for new treatments for spastic bladder and flaccid bladder.

Urinary sphincter muscles may also be affected by spinal cord injuries, resulting in a condition known as “dyssynergia.” Dyssynergia involves an inability of urinary sphincter muscles to relax when the bladder contracts, including active contraction in response to bladder contraction, which prevents urine from flowing through the urethra and results in the incomplete emptying of the bladder and “reflux” of urine into the kidneys. Traditional treatments for dyssynergia include medications that have been somewhat inconsistent in their efficacy or surgery. Therefore, the compounds of the present invention meet an existing need for new treatments for dyssynergia.

Peripheral vs. Central Effects

The mammalian nervous system comprises a central nervous system (CNS, comprising the brain and spinal cord) and a peripheral nervous system (PNS, comprising sympathetic, parasympathetic, sensory, motor, and enteric neurons outside of the brain and spinal cord). Where an active agent according to the present invention is intended to act centrally (i.e., exert its effects via action on neurons in the CNS), the active agent must either be administered directly into the CNS or be capable of bypassing or crossing the blood-brain barrier. The blood-brain barrier is a capillary wall structure that effectively screens out all but selected categories of substances present in the blood, preventing their passage into the CNS. The unique morphologic characteristics of the brain capillaries that make up the blood-brain barrier are: 1) epithelial-like high resistance tight junctions which literally cement all endothelia of brain capillaries together within the blood-brain barrier regions of the CNS; and 2) scanty pinocytosis or transendothelial channels, which are abundant in endothelia of peripheral organs. Due to the unique characteristics of the blood-brain barrier, many hydrophilic drugs and peptides that readily gain access to other tissues in the body are barred from entry into the brain or their rates of entry are very low.

The blood-brain barrier can be bypassed effectively by direct infusion of the active agent into the brain, or by intranasal administration or inhalation of formulations suitable for uptake and retrograde transport of the active agent by olfactory neurons.

The most common procedure for administration directly into the CNS is the implantation of a catheter into the ventricular system or intrathecal space. Alternatively, the active agent can be modified to enhance its transport across the blood-brain barrier. This generally requires some solubility of the drug in lipids, or other appropriate modification known to one of skill in the art. For example, the active agent may be truncated, derivatized, latentiated (converted from a hydrophilic drug into a lipid-soluble drug), conjugated to a lipophilic moiety or to a substance that is actively transported across the blood-brain barrier, or modified using standard means known to those skilled in the art. See, for example, Pardridge, Endocrine Reviews 7: 314-330 (1986) and U.S. Pat. No. 4,801,575.

Where an active agent according to the present invention is intended to act exclusively peripherally (i.e., exert its effects via action either on neurons in the PNS or directly on target tissues), it may be desirable to modify the compounds of the present invention such that they will not pass the blood-brain barrier. The principle of blood-brain barrier permeability can therefore be used to design active agents with selective potency for peripheral targets. Generally, a lipid-insoluble drug will not cross the blood-brain barrier, and will not produce effects on the CNS. A basic drug that acts on the nervous system may be altered to produce a selective peripheral effect by quaternization of the drug, which decreases its lipid solubility and makes it virtually unavailable for transfer to the CNS. For example, the charged antimuscarinic drug methscopalamine bromide has peripheral effects while the uncharged antimuscarinic drug scopolamine acts centrally. One of skill in the art can select and modify active agents of the present invention using well-known standard chemical synthetic techniques to add a lipid impermeable functional group such a quaternary amine, sulfate, carboxylate, phosphate, or sulfonium to prevent transport across the blood-brain barrier. Such modifications are by no means the only way in which active agents of the present invention may be modified to be impermeable to the blood-brain barrier; other well known pharmaceutical techniques exist and would be considered to fall within the scope of the present invention.

Agents

Compounds useful in the present invention include any active agent as defined elsewhere herein. Such active agents include, for example, sodium channel modulators, including TTX-R sodium channel modulators and/or activity dependent sodium channel modulators. TTX-R sodium channel modulators for use in the present invention include but are not limited to compounds that modulate or interact with Nav.1.8 and/or Naᵥ1.9 channels.

Voltage gated sodium channels, also known as voltage dependent sodium channels, are membrane-spanning proteins which permit controlled sodium influx from an extracellular environment into the interior of a cell. Opening and closing (gating) of voltage gated sodium channels is controlled by a voltage sensitive region of the protein containing charged amino acids that move within an electric field. The movement of these charged groups leads to conformational changes in the structure of the channel resulting in conducting (open/activated) or non-conducting (closed/inactivated) states.

Voltage gated sodium channels are present in a variety of tissues and are implicated in several vital processes in animals. Changes in sodium influx into cells mediated through voltage dependent sodium channels have been implicated in various human disorders such as epilepsy, pain, anaesthesia, neuroprotection, arrhythmia, and migraine (See, e.g., U.S. Pat. No. 6,479,498).

At least nine distinct voltage gated sodium channels have been identified in mammals (A. I. Goldin (2001) Annu. Rev. Physiol., 63: 871-94). Although most voltage gated sodium channels are tetrodotoxin-sensitive (TTX-S), tetrodotoxin-resistant (TTX-R) sodium channels have also been identified. Two of these TTX-R sodium channels, Naᵥ1.8 and Naᵥ1.9, are thought to be specific to sensory neurons, including neurons of the dorsal root ganglia (DRG). Antisense and knockout technologies have suggested a possible role for TTX-R sodium channels in painful bladder disorders (See e.g., N. Yoshimura et al. (2001) J. Neurosci. 21: 8690-6; N. Yoshimura et al. (2001) Urology 57: 116-7).

Compounds have been described that modulate sodium channels in an activity-dependent manner, meaning that these compounds preferentially modulate the activity of a sodium channel that has been activated or opened, and exhibit their effect either by modifying the activity of the open channel, or by modifying the activity of the inactivated state of the channel as described in Hille B. (1992) Ionic Channels in Excitable Membranes. 2nd ed. Sinauer Associates, Sunderland, Mass., pp. 390-422. Generally, this activity-dependent sodium channel modulation will alter the release of neurotransmitters under conditions that would normally cause sustained depolarization of neurons and/or repetitive firing of action potentials. Compounds that modulate sodium channels in an activity-dependent manner may include agents that interact with the sodium channel pore itself, as well as those that act as allosteric modulators of the channel by interacting with to a site on the channel complex.

Some sodium channel modulators may selectively modulate TTX-R sodium channels, while others may act non-selectively on sodium channels. Likewise, some activity dependent sodium channel modulators may selectively modulate TTX-R sodium channels, while others may act non-selectively on sodium channels, or on non-TTX-R sodium channels.

Agents useful in the practice of the invention include, but are not limited to propionamides such as Ralfinamide (NW-1029) (as disclosed in U.S. Pat. No. 5,236,957 and U.S. Pat. No. 5,391,577), which is also known as (+)-2(S)-[4-(2-Fluorobenzyloxy)benzylamino]propionamide and is represented by the following structure:

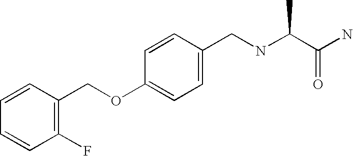

It is understood that the present invention also encompasses any salts, enantiomers, analogs, esters, amides, and derivatives of Ralfinamide, including:

a. Safinamide (as disclosed in U.S. Pat. No. 5,236,957 and U.S. Pat. No. 5,391,577), which is also known as 2(S)-[4-(3-Fluorobenzyloxy)benzylamino]propionamide methanesulfonate and is represented by the following structure:

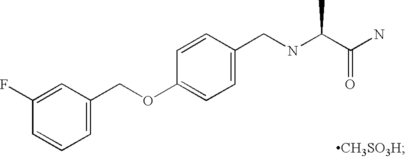

b. Other N-phenylalkyl substituted α-amino carboxamide derivatives in addition to Ralfinamide and Salfinamide as disclosed in U.S. Pat. No. 5,236,957;

c. Other N-phenylalkyl substituted α-amino carboxamide derivatives in addition to Ralfinamide and Salfinamide as disclosed in U.S. Pat. No. 5,391,577;

d. Substituted 2-benzylamino-2-phenyl-acetamide compounds as disclosed in U.S. Pat. No. 6,303,819, including agents with the following structural structure:

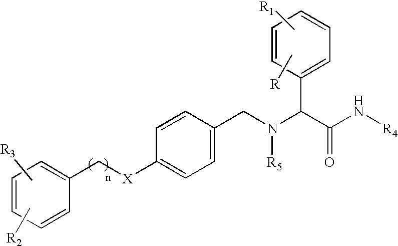

wherein:

n is zero, 1, 2, or 3;

X is —O—, —S—, —CH₂—, or —NH—;

each of R, R₁, R₂, and R₃, independently, is hydrogen, C₁-C₆ alkyl, halogen, hydroxyl, C₁-C₆ alkyl, halogen, hydroxyl, C₁-C₆ alkoxy, or trifluoromethyl;

each of R₄and R₅, independently, is hydrogen, C₁-C₆ alkyl or C₃-C₇ cycloalkyl; or a pharmaceutically acceptable salt thereof; and

e. 2-(4-Substituted)-benzylamino-2-methyl-propanamide derivatives as disclosed in U.S. Pat. No. 5,945,454, including agents with the following structural structure:

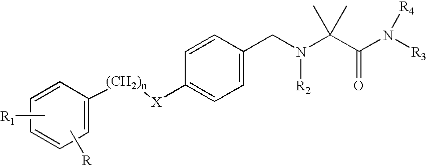

wherein:

n is zero, 1, 2,or3;

X is —O—, —S—, —CH₂—, or —NH—;

each or R and R₁ independently is hydrogen, C₁-C₆ alkyl, halogen, hydroxyl, C₁-C₄ alkoxy, or trifluoromethyl;

each of R₂, R₃, and R₄ independently is hydrogen, C₁-C₆ alkyl, or C₃-C₇ cycloalkyl; or

a pharmaceutically acceptable salt thereof with a proviso that when X is —S— and R, R₁, R₂, R₃, and R₄ are hydrogen, n is not zero.

It is further understood that the present invention also encompasses any salts, enantiomers, analogs, esters, amides, and derivatives of any of the aforementioned compounds.

Additional agents useful in the practice of the invention include, but are not limited to, aryldiazines and aryltriazines such as:

a. Sipatrigine (BW-619C; as disclosed in U.S. Pat. No. 5,684,005), which is also known as 4-Amino-2-(4-methylpiperazin-1-yl)-5-(2,3,5-trichlorophenyl)pyrimidine; 2-(4-Methylpiperazin-1-yl)-5 -(2,3,5-trichlorophenyl)pyrimidine-4-amine and is represented by the following structure:

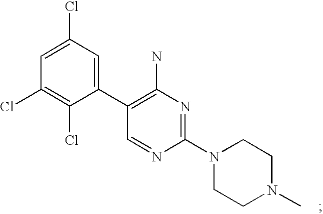

b. Lamotrigine (as disclosed in U.S. Pat. No. 4,602,017), which is also known as 6-(2,3-Dichlorophenyl)-1,2,4-triazine-3,5-diamine and is represented by the following structure:

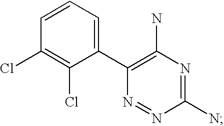

c. GW-273293 (as disclosed in U.S. Pat. No. 6,599,905), which is also known as 3-(2,3,5-Trichlorophenyl)pyrazine-2,6-diamine and is represented by the following structure:

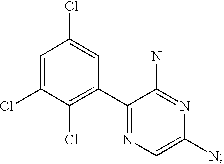

d. 4030W92 (as disclosed in U.S. Pat. No. 6,124,308), which is also known as 5-(2,3-Dichlorophenyl)-6-(fluoromethyl)pyrimidine-2,4-diamine and is represented by the following structure:

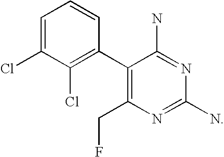

It is understood that the present invention also encompasses any salts, enantiomers, analogs, esters, amides, and derivatives of the aforementioned agents.

Additional agents useful in the practice of the invention include, but are not limited to, dibenzazepines such as:

a. Carbamazepine (as disclosed in U.S. Pat. No. 2,948,718), which is also known as 5H-Dibenz[d,f]azepine-5-carboxamide and is represented by the following structure:

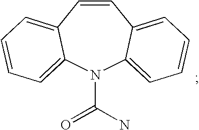

b. Oxcarbazepine (as disclosed in U.S. Pat. No. 3,642,775), which is also known as 10-Oxo-10,11-dihydro-5H-dibenz[b,f]azepine-5-carboxamide and is represented by the following structure:

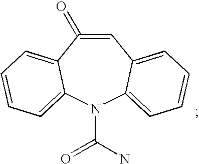

c. Licarbazepine (as disclosed in DE 2011045), which is also known as (±)-10-Hydroxy-10,11-dihydro-5H-dibenz[b,f]azepine-5-carboxamide and is represented by the following structure:

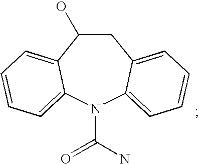

d. BIA-2-093 (as disclosed in U.S. Pat. No. 5,753,646), which is also known as Acetic acid 5-carbamoyl-10,11-dihydro-5H-dibenzo[b,f]azepin-10(S)-yl ester and is represented by the following structure:

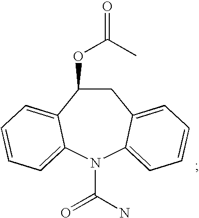

and

e. ADCI (as disclosed in U.S. Pat. No. 5,196,415), which is also known as (±)-5,10-Imino-10,11-dihydro-5H-dibenzo[a,d]cycloheptene-5-carboxamide and is represented by the following structure:

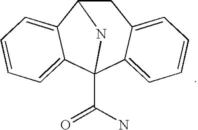

It is understood that the present invention also encompasses any salts, enantiomers, analogs, esters, amides, and derivatives of the aforementioned agents.

Additional agents useful in the practice of the invention include, but are not limited to, hydantoins such as:

a. Phenytoin sodium (as disclosed in U.S. Pat. No. 2,409,754) and OROS®-Phenytoin (as disclosed in U.S. Pat. No. 4,260,769), which are also known as 5,5-Diphenylhydantoin sodium salt and 5,5-Diphenyl-2,4-imidazolidinedione salt, respectively, and represented by the following structure:

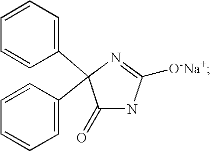

and

b. Fosphenytoin sodium (as disclosed in U.S. Pat. No. 4,260,769) and phosphenytoin sodium, which are also known as 3-(Hydroxymethyl)-5,5-diphenylhydantoin phosphate ester disodium salt and 5,5-Diphenyl-3-[(phosphonooxy)methyl]-2,4-imidazolidinedione disodium salt and are represented by the following structure:

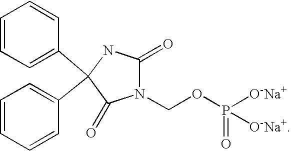

It is understood that the present invention also encompasses any salts, enantiomers, analogs, esters, amides, and derivatives of the aforementioned agents.

Additional agents useful in the practice of the invention include, but are not limited to, 3 and 4 atom spaced phenyl amines such as:

a. Pilsicainide hydrochloride and analogs thereof (as disclosed in U.S. Pat. No. 4,564,624), which is also known as N-(2,6-Dimethylphenyl)-8-pyrrolizidineacetamide hydrochloride; N-(2,6-Dimethylphenyl)-1-azabicyclo[3.3.0]octane-5-acetamide hydrochloride and is represented by the following structure:

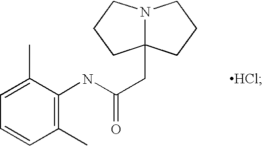

b. Tocainide (as disclosed in DE 2235745), which is also known as 2-Amino-N-(2,6-dimethylphenyl)propanamide hydrochloride and is represented by the following structure:

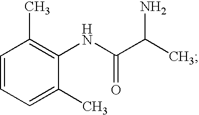

c. Flecainide (as disclosed in U.S. Pat. No. 3,900,481), which is also known as N-(2-Piperidylmethyl)-2,5-bis(2,2,2-trifluoroethoxy)benzamide monoacetate and is represented by the following structure:

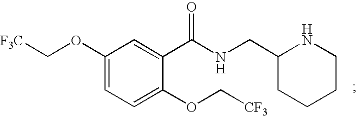

d. Mexiletine hydrochloride (as disclosed in U.S. Pat. No. 3,954,872), which is also known as 1-(2,6-Dimethylphenoxy)-2-propanamine hydrochloride and is represented by the following structure:

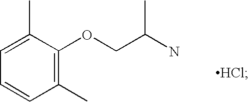

e. Ropivacaine hydrochloride (as disclosed in PCT Publication No. WO 85/00599), which is also known as (−)-(S)-N-(n-Propyl)piperidine-2-carboxylic acid 2,6-xylidide hydrochloride monohydrate; (−)-(S)-N-(2,6-Dimethylphenyl)-1-propylpiperidine-2-carboxamide hydrochloride monohydrate; (−)-(S)-1-Propyl-2′,6′-pipecoloxylidide hydrochloride monohydrate and is represented by the following structure:

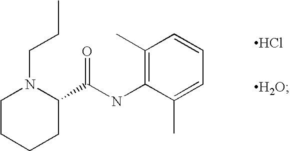

f. Lidocaine (as disclosed in U.S. Pat. No. 2,441,498), which is also known as 2-(diethylamino)-N-(2,6-dimethylphenyl)acetamide and is represented by the following structure:

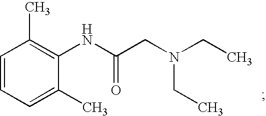

g. Mepivacaine (as disclosed in U.S. Pat. No. 2,799,679), which is also known as N-(2,6-dimethylphenyl)-1-methyl-2-piperidinecarboxamide and is represented by the following structure:

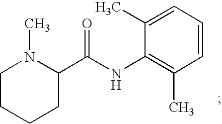

h. Bupivacaine (as disclosed in U.S. Pat. No. 2,955,111), which is also known as 1-butyl-N-(2,6-dimethylphenyl)-2-piperidinecarboxamide and is represented by the following structure:

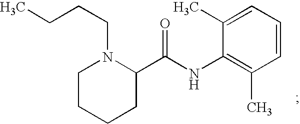

i. Prilocaine (as disclosed in U.S. Pat. No. 3,160,662), also known as N-(2-methylphenyl)-2-(propylamino)propanamide and is represented by the following structure:

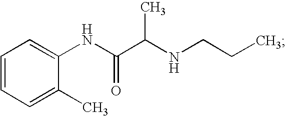

j. Etidocaine (as disclosed in U.S. Pat. No. 3,812,147), which is also known as N-(2,6-dimethylphenyl)-1-methyl-2-piperidinecarboxamide and is represented by the following structure:

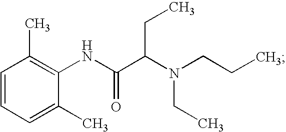

k. Tetracaine (as disclosed in U.S. Pat. No. 1,889,645), which is also known as 4-(butylamino)benzoic acid 2-(diethylamino)ethyl ester and is represented by the following structure:

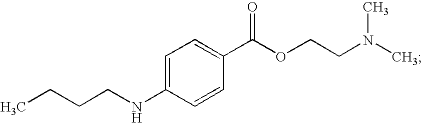

l. Dibucaine (as disclosed in U.S. Pat. No. 1,825,623), which is also known as 2-butoxy-N-[2-(diethylamino)-ethyl]-4-quinolinecarboxamide and is represented by the following structure:

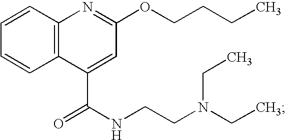

m. Soretolide, which is also known as 2,6-Dimethyl—N-(5-methylisozaxol-3-yl)benzamide and is represented by the following structure:

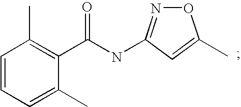

and

n. RS-132943 (as disclosed in U.S. Pat. No. 6,110,937), which is also known as 3(S)-(4-Bromo-2,6-dimethylphenoxymethyl)-1-methylpiperidine hydrochloride and is represented by the following structure:

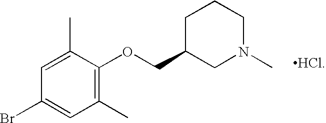

It is understood that the present invention also encompasses any salts, enantiomers, analogs, esters, amides, and derivatives of the aforementioned agents.

Additional agents useful in the practice of the invention include, but are not limited to, anticonvulsants such as:

a. Losigamone (as disclosed in U.S. Pat. No. 3,855,320), which is also known as (5R*)-5-[(alphaS*)-o-Chloro-alpha-hydroxybenzyl]-4-methoxy-2(5H)-furanone and is represented by the following structure:

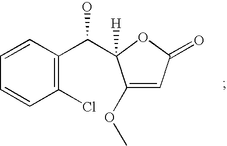

b. Zonisamide (as disclosed in U.S. Pat. No. 4,172,896), which is also known as 3-(Sulfamoylmethyl)-1,2-benzisoxazole; 1,2-Benzisoxazole-3-methanesulfonamide and is represented by the following structure:

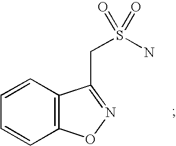

c. Topiramate (as disclosed in U.S. Pat. No. 4,513,006 ), which is also known as 2,3:4,5-Bis-O-(1-methylethylidene)-1-O-sulfamoyl-beta-D-fructopyranose; 2,3:4,5-Bis-O-(1-methylethylidene)-beta-D-fructopyranose sulfamate and is represented by the following structure:

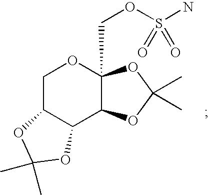

d. Rufinamide (as disclosed in U.S. Pat. No. 4,789,680), which is also known as 1-(2,6-Difluorobenzyl)-1H-1,2,3-triazole-4-carboxamide and is represented by the following structure:

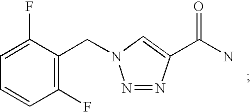

e. BW-534U87 (as disclosed in U.S. Pat. No. 5,166,209), which is also known as 4-Amino-1-(2,6-difluorobenzyl)-1H-1,2,3-triazolo[4,5-c]pyridine hydrochloride and is represented by the following structure:

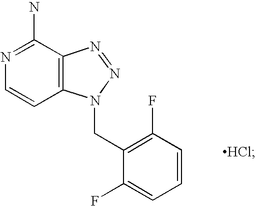

f. AWD-140-190 (as disclosed in U.S. Pat. No. 5,502,051), which is also known as 4-(4-Bromophenyl)-3-(morpholin-4-yl)pyrrole-2-carboxylic acid methyl ester and is represented by the following structure:

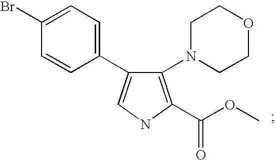

g. Harkoseride (as disclosed in U.S. Pat. No. 5,773,475), which is also known as erlosamide and 2(R)-Acetamido-N-benzyl-3-methoxypropionamide and is represented by the following structure:

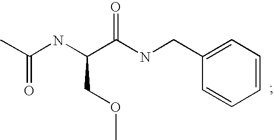

h. Memantine hydrochloride (as disclosed in U.S. Pat. No. 3,391,142) which is also known as 3,5-Dimethyl-1-adamantanamine hydrochloride and is represented by the following structure:

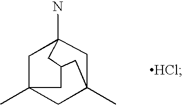

i. Felbamate (as disclosed in U.S. Pat. No. 2,884,444), which is also known as 2-Phenyl-1,3-propanediol dicarbamate and is represented by the following structure:

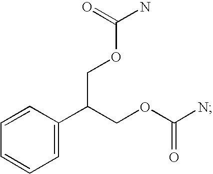

j. Valproate, which is also known as 2-Propylpentanoic acid sodium salt and is represented by the following structure:

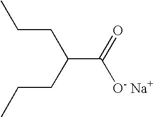

It is understood that the present invention also encompasses any salts, enantiomers, analogs, esters, amides, and derivatives of the aforementioned agents.

Additional agents useful in the practice of the invention include, but are not limited to, peptide toxins and/or insecticides such as:

a. μ-conotoxin SmIIIA from Conus stercusmuscarum as disclosed in West et al. (2002) Biochemistry 41:15388-15393;

b. Toxins as disclosed in Tan et al. (2001) Neuropharmacology 40:352-357;

c. Tarantula venom toxins ProTx-I and ProTx-II as disclosed in Middleton et al. (2002) Biochemistry 41:14734-14747;

d. Scorpion neurotoxin BmK IT2;

e. Pacific Ciguatoxin-1 (P-CTX-1);

f. Indoxacarb (as disclosed in WO 9211249), which is also known as methyl (S)-N-[7-chloro-2,3,4a,5-tetrahydro-4a-(methoxycarbonyl)indeno[1,2-e][1,3,4]oxadiazin-2-ylcarbonyl-4′-(trifluoromethoxy)carbanilate and is represented by the following structure:

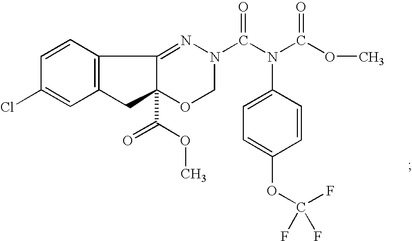

g. The DCJW metabolite of indoxacarb;

h. RH-3421 (as disclosed in Tsurubuchi et al., Neurotoxicology 22:443-453, 2001), which is also known as methyl 3-(4-chlorophenyl)-1-[N-(4-trifluoromethyl-phenyl)carbamoyl]-4-methyl-2-pyrazole-4-carboxylate and is represented by the following structure:

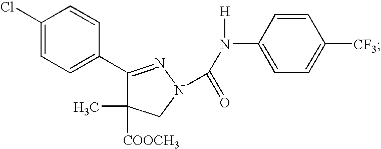

i. Deltamethrin (as disclosed in DE 2439177), which is also known as (S)-α-cyano-3-phenoxybenzyl (1R,3R)-3-(2,2-dibromovinyl)-2,2-dimethylcyclopropanecarboxylate and is represented by the following structure:

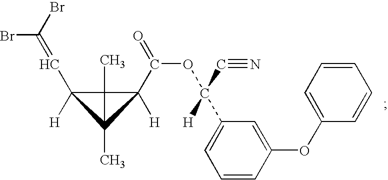

j. Tetramethrin (as disclosed in U.S. Pat. No. 3,268,398), which is also known as cyclonex-1-ene-1,2-dicarboximidomethyl (1RS,3RS;1RS,3SR)-2,2-dimethyl-3-(2-methylprop-1-enyl)cyclopropanecarboxylate and is represented by the following structure:

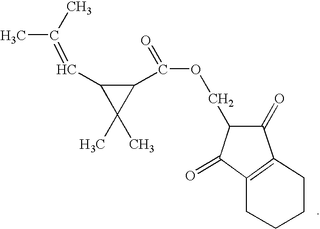

It is understood that the present invention also encompasses any salts, enantiomers, analogs, esters, amides, and derivatives of the aforementioned agents.

Additional agents useful in the practice of the invention include, but are not limited to:

a. Tetrodotoxin, which is also known as (4R,4aR,5R,7S,9S,10S,10aR,11S,12S)-Octahydro-12-(hydroxymethyl)-2-imino-5,9:7,10a-dimethano-10aH-[1,3]dioxocino[6,5-d]pyrimidine-4,7,10,11,12-pentol and is represented by the following structure:

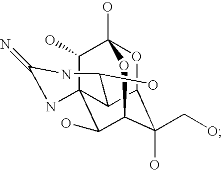

b. Ambroxol (as disclosed in U.S. Pat. No. 3,536,713), which is also known as 4-[[2-amino-3,5-dibromophenyl)methyl]amino]cyclohexanol and is represented by the following structure:

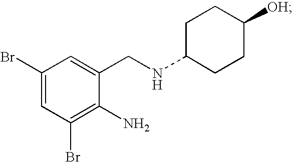

c. Enecadin hydrochloride (as disclosed in U.S. Pat. No. 6,191,149), which is also known as 4-(4-Fluorophenyl)-2-methyl-6-[5-(1-piperidinyl)pentyloxy]pyrimidine hydrochloride and is represented by the following structure:

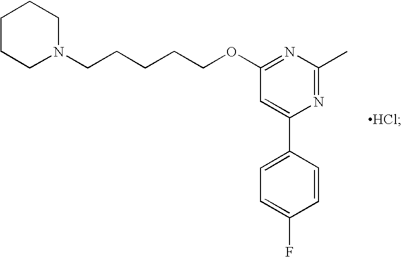

d. Fluphenazine hydrochloride (as disclosed in U.S. Pat. No. 3,058,979), which is also known as 4-[3-[2-(Trifluoromethyl)phenothiazin-10-yl]propyl]-1-piperazineethanol dihydrochloride and is represented by the following structure:

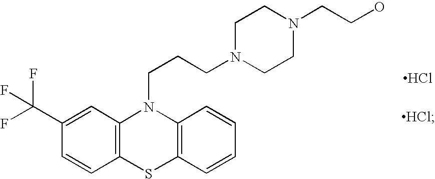

e. Trimebutine maleate (as disclosed in FR 1344455), which is also known as 3,4,5-Trimethoxybenzoic acid 2-(dimethylamino)-2-phenylbutyl ester maleate and is represented by the following structure:

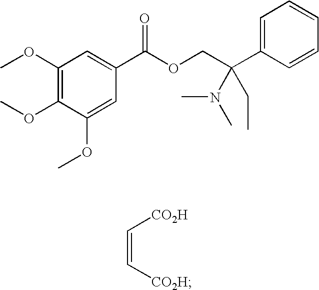

f. Riluzole (as disclosed in EP 0050551), which is also known as 2-Amino-6-(trifluoromethoxy)benzothiazole; 6-(Trifluoromethoxy)benzothiazol-2-amine and is represented by the following structure:

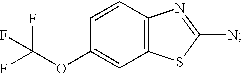

g. Silperisone hydrochloride (as disclosed in U.S. Pat. No. 5,198,446), which is also known as 1-(4-Fluorophenyl)-2,2-dimethyl-3-piperidino-2-silapropane hydrochloride; 1-[(4-Fluorobenzyl)dimethylsilylmethyl]piperidine hydrochloride and is represented by the following structure:

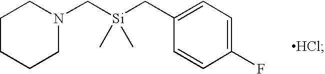

h. RSD-921 (as disclosed in U.S. Pat. No. 5,506,257), which is also known as (1R,2R)-N-Methyl-N-[2-(1-pyrrolidinyl)cyclohexyl]benzo[b]thiophene-4-acetamide and is represented by the following structure:

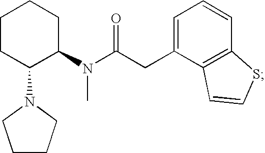

i. Crobenetine hydrochloride (as disclosed in U.S. Pat. No. 6,455,538), which is also known as (2R,6S)-3-[2(S)-Benzyloxypropyl]-6,11,11-trimethyl-1,2,3,4,5,6-hexahydro-2,6-methano-3-benzazocin-10-ol hydrochloride and is represented by the following structure:

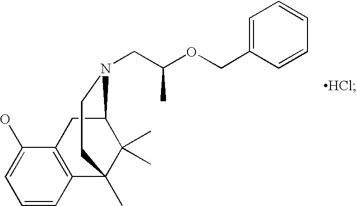

j. DL-017 (as disclosed in U.S. Pat. No. 5,340,814), which is also known as 3-[4-(2-Methoxyphenyl)piperazin-1-ylmethyl]-5-(methylsulfanyl)-2,3-dihydroimidazo[1,2-c]quinazoline and is represented by the following structure:

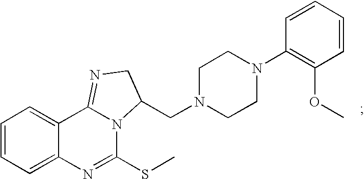

k. SUN—N8075 (as disclosed in U.S. Pat. No. 6,407,099), which is also known as 1-(4-Amino-2,3,5-trimethylphenoxy)-3-[4-[4-(4-fluorobenzyl)phenyl]piperazin-1-yl]propan-2(S)-ol dimethanesulfonate and is represented by the following structure:

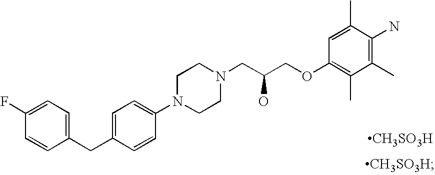

l. Amitriptyline (as disclosed in U.S. Pat. No. 3,205,264), which is also known as 3-(10,11-dihydro-5H-dibenzo [a,d]-cyclohepten-5-ylidene)-N,N-dimethyl-1-propanamine and is represented by the following structure:

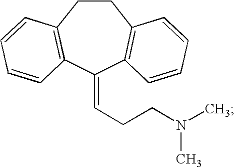

m. Compounds as disclosed in Oda et al. (2000) Anesth. Analg. 91:1213-1220;

n. Benzocaine, which is also known as 4-aminobenzoic acid ethyl ester, and is represented by the following structure:

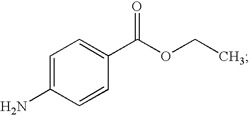

o. Compounds that inhibit the binding of Annexin II light chain or FHF1B to TTX-R sodium channels as disclosed in Liu et al., (2001) J. Biol. Chem. 276:18925-18933;

p. Thimerosal (as disclosed in U.S. Pat. No. 1,672,615), which is also known as ethyl[2-mercaptobenzoato(2-)-O,S]mercurate(1-) sodium and is represented by the following structure:

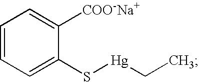

q. Vincamine, which is also known as (3α,14β,16α-14,15-dihydro-14-hydroxyebumamenine-14-carboxylic acid methyl ester and represented by the following structure:

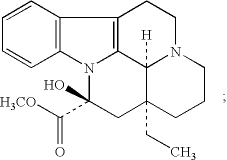

r. Quinidine, which is also known as 1(R)-(6-Methoxy-4-quinolinyl)-1-[(2R,4S,5R)-5-vinyl-1-azabicyclo[2.2.2]oct-2-yl]methanol and is represented by the following structure:

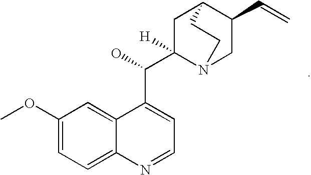

It is understood that the present invention also encompasses any salts, enantiomers, analogs, esters, amides, and derivatives of the aforementioned agents.

Other agents useful in the present invention include, but are not limited to, other compounds that interact with or modulate sodium channels, including synthetic peptides, peptidomimetics, or members of the same series or toxins from the same or related species as those compounds specifically listed above. Sodium channel modulators not intended for use in the present invention are tolperisone and vinpocetine. In addition, where the lower urinary tract disorder is OAB Wet, sodium channel modulators not intended for use in the present invention are semicarbazones and thiosemicarbazones, such as those claimed in U.S. patent application 20030225080.

The identification of other agents that have affinity for TTX-R sodium channels or proteins associated with TTX-R sodium channels and would be useful in the present invention can be determined by methods that measure functional TTX-R channel activity such as sodium flux as disclosed in Stallcup, WB (1979) J. Physiol. 286: 525-40 or electrophysiological approaches as disclosed in Weiser and Wilson (2002) Mol. Pharmacol. 62: 433-438. The identification of other agents that exhibit activity-dependent modulation of sodium channels and would be useful in the present invention can be determined by methods as disclosed in Li et al., (1999) Molecular Pharmacology 55:134-141.

One or more additional active agents can be administered with a sodium channel modulator, particularly a tetrodotoxin-resistant (TTX-R) sodium channel modulator and/or activity-dependent sodium channel modulator, either simultaneously or sequentially. The additional active agent will generally, although not necessarily, be one that is effective in treating overactive bladder, and/or an agent that augments the effect of the sodium channel modulator, particularly a tetrodotoxin-resistant (TTX-R) sodium channel modulator and/or activity-dependent sodium channel modulator. Suitable secondary agents include but are not limited to, for example, antispasmodics, tricyclic antidepressants, duloxetine, venlafaxine, monoamine reuptake inhibitors (including selective serotonin reuptake inhibitors (SSRI's) and serotonin/norepinephrin reuptake inhibitors (SNRI's)), spasmolytics, anticholinergics (particularly antimuscarinics), gabapentin, pregabalin, substituted aminomethyl-phenyl-cyclohexane derivatives including tramadol, 5-HT₃ antagonists, 5-HT₄ antagonists, β3 adrenergic agonists, neurokinin receptor antagonists, bradykinin receptor antagonists, nitric oxide donors and/or any agent that does not inhibit the action of the sodium channel modulator, particularly a tetrodotoxin-resistant (TTX-R) sodium channel modulator and/or activity-dependent sodium channel modulator.

Antispasmodic drugs that may be employed as additional active agents may include, for example, Alibendol, Ambucetamide, Aminopromazine, Apoatropine, Bevonium Methyl Sulfate, Bietamiverine, Butaverine, Butropium Bromide, N-Butylscopolammonium Bromide, Caroverine, Cimetropium Bromide, Cinnamedrine, Clebopride, Coniine Hydrobromide, Coniine Hydrochloride, Cyclonium Iodide, Difemerine, Diisopromine, Dioxaphetyl Butyrate, Diponium Bromide, Drofenine, Emepronium Bromide, Ethaverine, Feclemine, Fenalamide, Fenoverine, Fenpiprane, Fenpiverinium Bromide, Fentonium Bromide, Flavoxate, Flopropione, Gluconic Acid, Guaiactamine, Hydramitrazine, Hymecromone, Leiopyrrole, Mebeverine, Moxaverine, Nafiverine, Octamylamine, Octaverine, Pentapiperide, Phenamacide Hydrochloride, Phloroglucinol, Pinaverium Bromide, Piperilate, PipoxolanHydrochloride, Pramiverin, Prifinium Bromide, Properidine, Propivane, Propyromazine, Prozapine, Racefemine, Rociverine, Spasmolytol, Stilonium Iodide, Sultroponium, Tiemonium Iodide, Tiquizium Bromide, Tiropramide, Trepibutone, Tricromyl, Trifolium, Trimebutine, N,N-1Trimethyl-3,3-diphenyl-propylamine, Tropenzile, Trospium Chloride, and Xenytropium Bromide.

Spasmolytics are compounds that relieve, prevent, or lessen muscle spasms, especially of smooth muscle. In general, spasmolytics have been implicated as having efficacy in the treatment of visceral disorders (See. e.g., Takeda et al. (2000) J. Pharmacol. Exp. Ther. 293: 939-45).

Any spasmolytic agent is also useful as an additional active agent in the present invention. Compounds that have been identified as spasmolytic agents and are useful as an additional active agent in the present invention include, but are not limited to:

a. α-α-diphenylacetic acid-4-(N-methyl-piperidyl) esters as disclosed in U.S. Pat. No. 5,897,875;

b. Human and porcine spasmolytic polypeptides in glycosylated form and variants thereof as disclosed in U.S. Pat. No. 5,783,416;

c. Dioxazocine derivatives as disclosed in U.S. Pat. No. 4,965,259;

d. Quaternary 6,11-dihydro-dibenzo-[b,e]-thiepine-11-N-alkylnorscopine ethers as disclosed in U.S. Pat. No. 4,608,377;

e. Quaternary salts of dibenzo[1,4]diazepinones, pyrido-[1,4]benzodiazepinones, pyrido[1,5]benzodiazepinones as disclosed in U.S. Pat. No. 4,594,190;

f. Endo-8,8-dialkyl-8-azoniabicyclo (3.2.1) octane-6,7-exo-epoxy-3-alkyl-carboxylate salts as disclosed in U.S. Pat. No. 4,558,054;

g. Pancreatic spasmolytic polypeptides as disclosed in U.S. Pat. No. 4,370,317;

h. Triazinones as disclosed in U.S. Pat. No. 4,203,983;

i. 2-(4-Biphenylyl)-N-(2-diethylamino alkyl)propionamide as disclosed in U.S. Pat. No. 4,185,124;

j. Piperazino-pyrimidines as disclosed in U.S. Pat. No. 4,166,852;

k. Aralkylamino carboxylic acids as disclosed in U.S. Pat. No. 4,163,060;

l. Aralkylamino sulfones as disclosed in U.S. Pat. No. 4,034,103;

m. Smooth muscle spasmolytic agents as disclosed in U.S. Pat. No. 6,207,852; and

n. papaverine.

The identification of further compounds that have spasmolytic activity and would therefore be useful as an additional active agent in the present invention can be determined by performing bladder strip contractility studies as described in U.S. Pat. No. 6,207,852; Noronha-Blob et al. (1991) J. Pharmacol. Exp. Ther.256: 562-567; and/or Kachur et al. (1988) J. Pharmacol. Exp. Ther.247: 867-872.

Acetylcholine is a chemical neurotransmitter in the nervous systems of all animals. “Cholinergic neurotransmission” refers to neurotransmission that involves acetylcholine, and has been implicated in the control of functions as diverse as locomotion, digestion, cardiac rate, “fight or flight” responses, and learning and memory (Salvaterra (February 2000) Acetylcholine. In Encyclopedia of Life Sciences. London: Nature Publishing Group, http:/www.els.net). Receptors for acetylcholine are classified into two general categories based on the plant alkaloids that preferentially bind to them: 1) nicotinic (nicotine binding); or 2) antimuscarinic (muscarine binding) (See, e.g., Salvaterra, Acetylcholine, supra).

The two general categories of acetylcholine receptors may be further divided into subclasses based upon differences in their pharmacological and electrophysiological properties. Nicotinic receptors are ligand gated ion channels composed of a variety of subunits that are used to identify the following subclasses: 1) muscle nicotinic acetylcholine receptors; 2) neuronal nicotinic acetylcholine receptors that do not bind the snake venom α-bungarotoxin; and 3) neuronal nicotinic acetylcholine receptors that do bind the snake venom α-bungarotoxin (Dani et al. (July 1999) Nicotinic Acetylcholine Receptors in Neurons. In Encyclopedia of Life Sciences. London: Nature Publishing Group, http:/www.els.net; Lindstrom (October 2001) Nicotinic Acetylcholine Receptors. In Encyclopedia of Life Sciences. London: Nature Publishing Group, http:/www.els.net). By contrast, muscarinic receptors may be divided into five subclasses, labeled M₁-M₅, and preferentially couple with specific G-proteins (M₁, M₃, and M₅ with G_{q}; M₂ and M₄ with Gᵢ/Gₒ) (Nathanson (July 1999) Muscarinic Acetylcholine Receptors. In Encyclopedia of Life Sciences. London: Nature Publishing Group, http:/www.els.net). In general, muscarinic receptors have been implicated in smooth muscle function (See, e.g., Appell (2002) Cleve. Clin. J. Med. 69: 761-9; Diouf et al. (2002) Bioorg. Med. Chem. Lett. 12: 2535-9; Crandall (2001) J. Womens Health Gend. Based Med. 10: 735-43; Chapple (2000) Urology 55: 33-46).

Any anticholinergic agent, specifically, any antimuscarinic agent, is useful as an additional active agent in the present invention. Compounds that have been identified as antimuscarinic agents and are useful as an additional active agent in the present invention include, but are not limited to:

a. Darifenacin (Daryon®);

b. YM-905 (solifenacin succinate);

c. Oxybutynin (Ditropan®);

d. S-Oxybutynin;

e. N-desethyl-oxybutynin;

f. Tolterodine (Detrol®);

g. Trospium (Uraplex®, Spasmex®);

h. Propiverine (Detrunorm®);

i. Propantheline bromide (Pro-Banthine®);

j. Hyoscyamine sulfate (Levsin®, Cystospaz®);

k. Dicyclomine hydrochloride (Bentyl®);

l. Flavoxate hydrochloride (Urispas®);

m. d,l (racemic) 4-diethylamino-2-butynyl phenylcyclohexylglycolate;

n. (R)—N,N-diisopropyl-3-(2-hydroxy-5-methylphenyl)-3-phenylpropanamine L-hydrogen tartrate;

o. (+)-(1S,3′R)-quinuclidin-3′-yl 1-phenyl-1,2,3,4-tetrahydroisoquinoline-2-carboxylate monosuccinate;

p. alpha(+)-4-(Dimethylamino)-3-methyl-1,2-diphenyl-2-butanol proprionate;

q. 1-methyl-4-piperidyl diphenylpropoxyacetate;

r. 3″-hydroxyspiro[1″H,5″H-nortropane-8,1′-pyrrolidinium benzilate;

s. 4 amino-piperidine containing compounds as disclosed in Diouf et al. (2002) Bioorg. Med. Chem. Lett. 12: 2535-9;

t. pirenzipine;

u. methoctramine;

v. 4-diphenylacetoxy-N-methyl piperidine methiodide;

w. tropicamide;

x. (2R)-N-[1-(6-aminopyridin-2-ylmethyl)piperidin-4-yl]-2-[(1R)-3,3-difluorocyclopentyl]-2-hydroxy-2-phenylacetamide;

y. PNU-200577 ((R)—N, N-diisopropyl-3-(2-hydroxy-5-hydroxymethylphenyl)-3-phenylpropanamine); and

z. NS-21

The identification of further compounds that have antimuscarinic activity and would therefore be useful as an additional active agent in the present invention can be determined by performing muscarinic receptor binding specificity studies as described by Nilvebrant (2002) Pharmacol. Toxicol. 90: 260-7 or cystometry studies as described by Modiri et al. (2002) Urology 59: 963-8.

Adrenergic receptors are cell-surface receptors for two major catecholamine hormones and neurotransmitters: noradrenaline and adrenaline. (Malbon et al. (February 2000) Adrenergic Receptors. In Encyclopedia of Life Sciences. London: Nature Publishing Group, http:/www.els.net). Adrenergic receptors have been implicated in critical physiological processes, including blood pressure control, myocardial and smooth muscle contractility, pulmonary function, metabolism, and central nervous system activity (See, e.g., Malbon et al., Adrenergic Receptors, supra). Two classes of adrenergic receptors have been identified, α and β, that may be further subdivided into three major families (α1, α2, and β), each with at least three subtypes (α1A, B, and, D; α2A, B, and C; and β1, β2, and β3) based upon their binding characteristics to different agonists and molecular cloning techniques. (See, e.g., Malbon et al., Adrenergic Receptors, supra). It has been shown that β3 adrenergic receptors are expressed in the detrusor muscle, and that the detrusor muscle relaxes with a β3-agonist (Takeda, M. et al. (1999) J.Pharmacol.Exp.Ther. 288: 1367-1373), and in general, β3 adrenergic receptors have been implicated in bladder function (See, e.g., Takeda et al. (2002) Neuourol. Urodyn. 21: 558-65; Takeda et al. (2000) J. Pharmacol. Exp. Ther. 293: 939-45.

Other agents useful in the present invention include any β3 adrenergic agonist agent. Compounds that have been identified as β3 adrenergic agonist agents and are useful in the present invention include, but are not limited to:

a. TT-138 and phenylethanolamine compounds as disclosed in U.S. Pat. No. 6,069,176, PCT Publication No. WO 97/15549 and available from Mitsubishi Pharma Corp.;

b. FR-149174 and propanolamine derivatives as disclosed in U.S. Pat. Nos. 6,495,546 and 6,391,915 and available from Fujisawa Pharmaceutical Co.;

c. KUC-7483, available from Kissei Pharmaceutical Co.,

d. 4′-hydroxynorephedrine derivatives such as 2-2-chloro-4-(2-((1S,2R)-2-hydroxy-2-(4-hydroxyphenyl)-1-methylethylamino)ethyl)phenoxy acetic acid as disclosed in Tanaka et al. (2003) J. Med. Chem. 46: 105-12;

e. 2-amino-1-phenylethanol compounds, such as BRL35135 ((R*R*)-(.+-.)-[4-[2-[2-(3-chlorophenyl)-2-ydroxyethylamino]propyl]phenoxy]acetic acid methyl ester hydrobromide salt as disclosed in Japanese Patent Publication No. 26744 of 1988 and European Patent Publication No. 23385), and SR58611A ((RS)-N-(7-ethoxycarbonylmethoxy-1,2,3,4-tetrahydronaphth-2-yl)-2-(3-chlorophenyl)-2-hydroxyethanamine hydrochloride as disclosed in Japanese Laid-open Patent Publication No. 66152 of 1989 and European Laid-open Patent Publication No. 255415);

f. GS 332 (Sodium (2R)-[3-[3-[2-(3 Chlorophenyl)-2-hydroxyethylamino]cyclohexyl]phenoxy]acetate) as disclosed in Iizuka et al. (1998) J. Smooth Muscle Res. 34: 139-49;

g. BRL-37,344 (4-[-[(2-hydroxy-(3-chlorophenyl) ethyl)-amino]propyl]phenoxyacetate) as disclosed in Tsujii et al. (1998) Physiol. Behav. 63: 723-8 and available from Glaxosmithkline;

h. BRL-26830A as disclosed in Takahashi et al. (1992) Jpn Circ. J. 56: 936-42 and available from Glaxosmithkline;

i. CGP 12177 (4-[3-t-butylamino-2-hydroxypropoxy]benzimidazol-2-one) (a β1/β2 adrenergic antagonist reported to act as an agonist for the β3 adrenergic receptor) as described in Tavernier et al. (1992) J. Pharmacol. Exp. Ther. 263: 1083-90 and available from Ciba-Geigy;

j. CL 316243 (R,R-5-[2-[[2-(3-chlorophenyl)-2-hydroxyethyl]amino]propyl]-1,3-benzodioxole-2,2-dicarboxylate) as disclosed in Berlan et al. (1994) J. Pharmacol. Exp. Ther. 268: 1444-51;

k. Compounds having β3 adrenergic agonist activity as disclosed in U.S. patent application 20030018061;

l. ICI 215,001 HCl ((S)-4-[2-Hydroxy-3-phenoxypropylaminoethoxy]phenoxyacetic acid hydrochloride) as disclosed in Howe (1993) Drugs Future 18: 529 and available from AstraZeneca/ICI Labs;

m. ZD 7114 HCl (ICI D7114; (S)-4-[2-Hydroxy-3-phenoxypropylaminoethoxy]-N-(2-methoxyethyl)phenoxyacetamide HCl) as disclosed in Howe (1993) Drugs Future 18: 529 and available from AstraZeneca/ICI Labs;

n. Pindolol (1-(1H-Indol-4-yloxy)-3-[(1-methylethyl)amino]-2-propanol) as disclosed in Blin et al (1994) Mol.Pharmacol. 44: 1094;

o. (S)-(−)-Pindolol ((S)-1-(1H-indol-4-yloxy)-3-[(1-methylethyl)amino]-2-propanol) as disclosed in Walter et al (1984) Naunyn-Schmied.Arch.Pharmacol. 327: 159 and Kalkman (1989) Eur.J.Pharmacol. 173: 121;

p. SR 59230A HCl (1-(2-Ethylphenoxy)-3-[[(1S)-1,2,3,4-tetrahydro-1-naphthalenyl]amino]-(2S)-2-propanol hydrochloride) as disclosed in Manara et al. (1995) Pharmacol. Comm. 6: 253 and Manara et al. (1996) Br. J. Pharmacol. 117: 435 and available from Sanofi-Midy; and

q. SR 58611 (N[2s)7-carb-ethoxymethoxy-1,2,3,4-tetra-hydronaphth]-(2r)-2-hydroxy-2(3-chlorophenyl) ethamine hydrochloride) as disclosed in Gauthier et al. (1999) J. Pharmacol. Exp. Ther. 290: 687-693 and available from Sanofi Research.

The identification of further compounds that have β3 adrenergic agonist activity and would therefore be useful in the present invention can be determined by performing radioligand binding assays and/or contractility studies as described by Zilberfarb et al. (1997) J. Cell Sci. 110: 801-807; Takeda et al. (1999) J. Pharmacol. Exp. Ther. 288: 1367-1373; and Gauthier et al. (1999) J. Pharmacol. Exp. Ther. 290: 687-693.

Tachykinins (TKs) are a family of structurally related peptides that include substance P, neurokinin A (NKA) and neurokinin B (NKB). Neurons are the major source of TKs in the periphery. An important general effect of TKs is neuronal stimulation, but other effects include endothelium-dependent vasodilation, plasma protein extravasation, mast cell recruitment and degranulation and stimulation of inflammatory cells (See Maggi, C. A. (1991) Gen. Pharmacol., 22: 1-24). In general, tachykinin receptors have been implicated in bladder function (See, e.g., Kamo et al. (2000) Eur. J. Pharmacol. 401: 235-40 and Omhura et al. (1997) Urol. Int. 59: 221-5).

Substance P activates the neurokinin receptor subtype referred to as NK₁. Substance P is an undecapeptide that is present in sensory nerve terminals. Substance P is known to have multiple actions that produce inflammation and pain in the periphery after C-fiber activation, including vasodilation, plasma extravasation and degranulation of mast cells (Levine, J. D. et. al. (1993) J. Neurosci. 13: 2273).

Neurokinin A is a peptide which is colocalized in sensory neurons with substance P and which also promotes inflammation and pain. Neurokinin A activates the specific neurokinin receptor referred to as NK₂ (Edmonds-Alt, S., et. al. (1992) Life Sci. 50: PL101). In the urinary tract, TKs are powerful spasmogens acting through only the NK₂ receptor in the human bladder, as well as the human urethra and ureter (Maggi, C. A. (1991) Gen. Pharmacol., 22: 1-24).

Other agents useful in the present invention include any neurokinin receptor antagonist agent. Suitable neurokinin receptor antagonists for use in the present invention that act on the NK₁ receptor include, but are not limited to: 1-imino-2-(2-methoxy-phenyl)-ethyl)-7,7-diphenyl-4-perhydroisoindolone(3aR ,7aR) (“RP 67580”); 2S,3S-cis-3-(2-methoxybenzylamino)-2-benzhydrylquinuclidine (“CP 96,345”); and (aR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione)(“TAK-637”). Suitable neurokinin receptor antagonists for use in the present invention that act on the NK₂ receptor include but are not limited to: ((S)-N-methyl—N-4-(4-acetylamino-4-phenylpiperidino)-2-(3,4-dichloropheny l)butylbenzamide (“SR 48968”); Met-Asp-Trp-Phe-Dap-Leu (“MEN 10,627”); and cyc(Gln-Trp-Phe-Gly-Leu-Met) (“L 659,877”). The identification of further compounds that have neurokinin receptor antagonist activity and would therefore be useful in the present invention can be determined by performing binding assay studies as described in Hopkins et al. (1991) Biochem. Biophys. Res. Comm. 180: 1110-1117; and Aharony et al. (1994) Mol. Pharmacol. 45: 9-19.

Bradykinin receptors generally are divided into bradykinin₁ (B₁) and bradykinin₂ (B₂) subtypes. Studies have shown that acute peripheral pain and inflammation produced by bradykinin are mediated by the B₂ subtype whereas bradykinin-induced pain in the setting of chronic inflammation is mediated via the B₁ subtype (Perkins, M. N., et. al. (1993) Pain 53: 191-97); Dray, A., et. al. (1993) Trends Neurosci. 16: 99-104). In general, bradykinin receptors have been implicated in bladder function (See, e.g., Meini et al. (2000) Eur. J. Pharmacol. 388: 177-82 and Belichard et al. (1999) Br. J. Pharmacol. 128: 213-9).

Other agents useful in the present invention include any bradykinin receptor antagonist agent. Suitable bradykinin receptor antagonists for use in the present invention that act on the B₁ receptor include but are not limited to: des-arg¹⁰HOE 140 (available from Hoechst Pharmaceuticals) and des-Arg⁹bradykinin (DABK). Suitable bradykinin receptor antagonists for use in the present invention that act on the B₂ receptor include but are not limited to: D-Phe⁷-BK; D-Arg-(Hyp³-Thi^{5,8}-D-Phe⁷)-BK (“NPC 349”); D-Arg-(Hyp³-D-Phe⁷)-BK (“NPC 567”); D-Arg-(Hyp³-Thi⁵-D-Tic⁷-Oic⁸)-BK (“HOE 140”); H-DArg-Arg-Pro-Hyp-Gly-Thi-c(Dab-DTic-Oic-Arg)c(7gamma-10alpha)(“MEN11270”); H-DArg-Arg-Pro-Hyp-Gly-Thi-Ser-DTic-Oic-Arg-OH(“Icatibant”); (E)-3-(6-acetamido-3-pyridyl)-N-[N-[2,4-dichloro-3-[(2-methyl-8-quinolinyl)oxymethyl]phenyl]-N-methylaminocarbonylmethyl]acrylamide (“FRI73567”); and WIN 64338. These compounds are more fully described in Perkins, M. N., et. al., Pain, supra; Dray, A., et. al., Trends Neurosci., supra; and Meini et al. (2000) Eur. J. Pharmacol. 388: 177-82. The identification of further compounds that have bradykinin receptor antagonist activity and would therefore be useful in the present invention can be determined by performing binding assay studies as described in Manning et al. (1986) J. Pharmacol. Exp. Ther. 237: 504 and U.S. Pat. No. 5,686,565.

Nitric oxide donors may be included in the present invention particularly for their anti-spasm activity. Nitric oxide (NO) plays a critical role as a molecular mediator of many physiological processes, including vasodilation and regulation of normal vascular tone. The action of NO is implicated in intrinsic local vasodilation mechanisms. NO is the smallest biologically active molecule known and is the mediator of an extraordinary range of physiological processes (Nathan (1994) Cell 78: 915-918; Thomas (1997) Neurosurg. Focus 3: Article 3). NO is also a known physiologic antagonist of endothelin-1, which is the most potent known mammalian vasoconstrictor, having at least ten times the vasoconstrictor potency of angiotensin II (Yanagisawa et al. (1988) Nature 332: 411-415; Kasuya et al. (1993) J. Neurosurg. 79: 892-898; Kobayashi et al., (1991) Neurosurgery 28: 673-679). The biological half-life of NO is extremely short (Morris et al. (1994) Am. J. Physiol. 266: E829-E839; Nathan (1994) Cell 78: 915-918). NO accounts entirely for the biological effects of endothelium-derived relaxing factor (EDRF) and is an extremely potent vasodilator that is believed to work through the action of cGMP-dependent protein kinases to effect vasodilation (Henry et al. (1993) FASEB J. 7: 1124-1134; Nathan (1992) FASEB J. 6: 3051-3064; Palmer et al., (1987) Nature 327: 524-526; Snyder et al. (1992) Scientific American 266: 68-77).

Within endothelial cells, an enzyme known as NO synthase (NOS) catalyzes the conversion of L-arginine to NO which acts as a diffusible second messenger and mediates responses in adjacent smooth muscle cells. NO is continuously formed and released by the vascular endothelium under basal conditions which inhibits contractions and controls basal coronary tone and is produced in the endothelium in response to various agonists (such as acetylcholine) and other endothelium dependent vasodilators. Thus, regulation of NOS activity and the resultant levels of NO are key molecular targets controlling vascular tone (Muramatsu et. al. (1994) Coron. Artery Dis. 5: 815-820).

Other agents useful in the present invention include any nitric oxide donor agent. Suitable nitric oxide donors for the practice of the present invention include but are not limited to:

a. Nitroglycerin;

b. Sodium nitroprusside;

c. FK 409 (NOR-3);

d. FR 144420 (NOR-4);

e. 3-morpholinosydnonimine;

f. Linsidomine chlorohydrate (“SIN-1”);

g. S-nitroso-N-acetylpenicillamine (“SNAP”);

h. AZD3582 (CINOD lead compound, available from NicOx S.A.);

i. NCX 4016 (available from NicOx S.A.);

j. NCX 701 (available from NicOx S.A.);

k. NCX 1022 (available from NicOx S.A.);

l. HCT 1026 (available from NicOx S.A.);

m. NCX 1015 (available from NicOx S.A.);

n. NCX 950 (available from NicOx S.A.);

o. NCX 1000 (available from NicOx S.A.);

p. NCX 1020 (available from NicOx S.A.);

q. AZD 4717 (available from NicOx S.A.);

r. NCX 151O/NCX 1512 (available from NicOx S.A.);

s. NCX 2216 (available from NicOx S.A.);

t. NCX 4040 (available from NicOx S.A.);

u. Nitric oxide donors as disclosed in U.S. Pat. No. 5,155,137;

v. Nitric oxide donors as disclosed in U.S. Pat. No. 5,366,997;

w. Nitric oxide donors as disclosed in U.S. Pat. No. 5,405,919;

x. Nitric oxide donors as disclosed in U.S. Pat. No. 5,650,442;

y. Nitric oxide donors as disclosed in U.S. Pat. No. 5,700,830;

z. Nitric oxide donors as disclosed in U.S. Pat. No. 5,632,981;

aa. Nitric oxide donors as disclosed in U.S. Pat. No. 6,290,981;

bb. Nitric oxide donors as disclosed in U.S. Pat. No. 5,691,423;

cc. Nitric oxide donors as disclosed in U.S. Pat. No. 5,721,365;

dd. Nitric oxide donors as disclosed in U.S. Pat. No.5,714,511;

ee. Nitric oxide donors as disclosed in U.S. Pat. No. 6,511,911; and

ff. Nitric oxide donors as disclosed in U.S. Pat. No. 5,814,666.

The identification of further compounds that have nitric oxide donor activity and would therefore be useful in the present invention can be determined by release profile and/or induced vasospasm studies as described in U.S. Pat. Nos. 6,451,337 and 6,358,536, as well as Moon (2002) IBJU Int. 89: 942-9 and Fathian-Sabet et al. (2001) J. Urol. 165: 1724-9.

Gabapentin (Neurontin, or 1-(aminomethyl) cyclohexaneacetic acid) is an anticonvulsant drug with a high binding affinity for some calcium channel subunits, and is represented by the following structure:

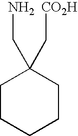

Gabapentin is one of a series of compounds of formula:

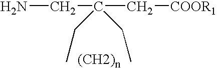

in which R₁ is hydrogen or a lower alkyl radical and n is 4, 5, or 6. Although gabapentin was originally developed as a GABA-mimetic compound to treat spasticity, gabapentin has no direct GABAergic action and does not block GABA uptake or metabolism. (For review, see Rose et al. (2002) Analgesia 57:451-462). Gabapentin has been found, however, to be an effective treatment for the prevention of partial seizures in patients who are refractory to other anticonvulsant agents (Chadwick (1991) Gabapentin, In Pedley T A, Meldrum B S (eds.), Recent Advances in Epilepsy, Churchill Livingstone, New York, pp. 211-222). Gabapentin and the related drug pregabalin interact with the α₂δ subunit of calcium channels (Gee et al. (1996) J. Biol. Chem. 271: 5768-5776).

In addition to its known anticonvulsant effects, gabapentin has been shown to block the tonic phase of nociception induced by formalin and carrageenan, and exerts an inhibitory effect in neuropathic pain models of mechanical hyperalgesia and mechanical/thermal allodynia (Rose et al. (2002) Analgesia 57: 451-462). Double-blind, placebo-controlled trials have indicated that gabapentin is an effective treatment for painful symptoms associated with diabetic peripheral neuropathy, post-herpetic neuralgia, and neuropathic pain (see, e.g., Backonja et al. (1998) JAMA 280:1831-1836; Mellegers et al. (2001) Clin. J. Pain 17:284-95).

Pregabalin, (S)-(3-aminomethyl)-5-methylhexanoic acid or (S)-isobutyl GABA, is another GABA analog whose use as an anticonvulsant has been explored (Bryans et al. (1998) J. Med. Chem. 41:1838-1845). Pregabalin has been shown to possess even higher binding affinity for the α₂δ subunit of calcium channels than gabapentin (Bryans et al. (1999) Med. Res. Rev. 19:149-177).

The substituted aminomethyl-phenyl-cyclohexane derivatives suitable for use in the invention are represented by structural Formula I:

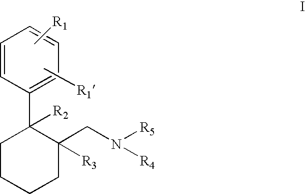

and enantiomers and mixtures thereof wherein:

R₁ and R₁′ are independently hydrogen, an aliphatic group, an aryl group, an arylalkyl group, a halogen, —CN, —OR₆, —SR₆, —NR₆R₆, —OC(O)R₆, —C(O)OR₆, —C(O)R₆ or —C(O)NR₆R₆;

R₂ is hydrogen, halogen, —OR₇ or —OC(O)R₇;

R₃ is hydrogen or an aliphatic group;

or R₂ and R₃ together form a double bond;

R₄ and R₅ are independently hydrogen, an aliphatic group, an aryl group, or an arylalkyl group;

R₆ is hydrogen, an aliphatic group, an aryl group or an arylalkyl group;

R₇ is hydrogen, an aliphatic group, an aryl group or an arylalkyl group;

or pharmaceutically acceptable salts, solvates or hydrates thereof.

In a particular embodiment of Formula I, R₂ is —OH. When R₂ is —OH, it is preferred that R₁′ is hydrogen and R₁ is OCH₃, preferably substituted at the meta position of the phenyl ring.

In a further embodiment of Formula I, R₂ is —OH, R₁ ′ is hydrogen and R₁ is —OR₆, substituted at the meta position of the phenyl ring and R₆ is an aliphatic group, for example, and alkyl group. In a particular embodiment, wherein R₂ is —OH, R₁′ is hydrogen and R₁ is —OR₆, substituted at the meta position of the phenyl ring and R₆ is an alkyl group, R₃, R₄ and R₅ can be hydrogen or an alkyl group.

In one embodiment, the substituted aminomethyl-phenyl-cyclohexane derivative

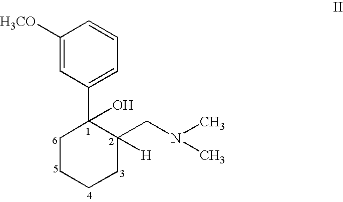

suitable for use in the invention is represented by structural Formula II:

and enantiomers and mixtures thereof or pharmaceutically acceptable salts, solvates or hydrates thereof.

In a particular embodiment, the compound of Formula II is a mixture of the (+)cis and (−)cis enantiomers, wherein the C-1 and C-2 carbons of the cyclohexyl ring are (1R,2R) and (1S,2S), respectively, and the substituents on C-1 and C-2 are in the cis orientation.

In a specific embodiment, the mixture of the (+)cis and (−)cis enantiomers is a racemic mixture. That is, the compound of Formula II is a 50:50 mixture of (+)cis and (−)cis enantiomers as shown below:

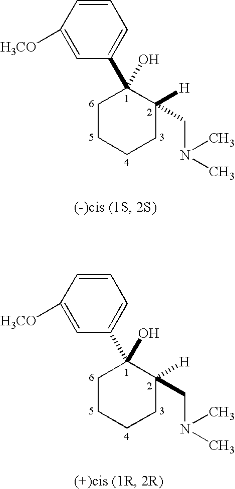

In other words, the compound of Formula II is the 50:50 mixture of (+/−)cis-2-[(dimethylamino)methyl]-1-(3-methoxyphenyl) cyclohexanol, commonly referred to as tramadol. The compound can be in the form of a pharmaceutically acceptable salt. Typically, tramadol is administered in the form of the hydrochloride salt. The tramadol hydrochloride is also known, for example, by the tradename ULTRAM®.

Tramadol in the form of the hydrochloride salt, is widely used as an analgesic. Tramadol is a centrally acting analgesic with a low affinity for opioid receptors. In contrast to other opioids, the analgesic action of tramadol is only partially inhibited by the opioid antagonist naloxone, which suggests the existence of an additional non-opioid mechanism of action. It has been found that monoaminergic activity, wherein noradrenaline and serotonin (5-HT) reuptake are inhibited, contributes significantly to the analgesic action of tramadol by blocking nociceptive impulses at the spinal level.

In a further embodiment, the administered compound is the (+)cis enantiomer of tramadol, set forth above.

In another embodiment, the substituted aminomethyl-phenyl-cyclohexane derivative is represented by the following structural Formula III in which the nitrogen of the aminomethyl group is in the form of the N-oxide:

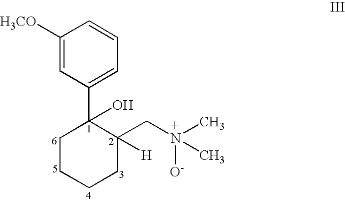

and enantiomers and mixtures thereof or pharmaceutically acceptable salts, solvates and hydrates thereof.

In a particular embodiment, the compound of Formula III is a mixture of the (+)cis and (−)cis enantiomers, wherein the C-1 and C-2 carbons of the cyclohexyl ring are (1R,2R) and (1S,2S), respectively, and the substituents on C-1 and C-2 are in the cis orientation.

In a specific embodiment, the mixture of the (+)cis and (−)cis enantiomers is a racemic mixture. That is, the compound of Formula III is a 50:50 mixture of (+)cis and (−)cis enantiomers as shown below:

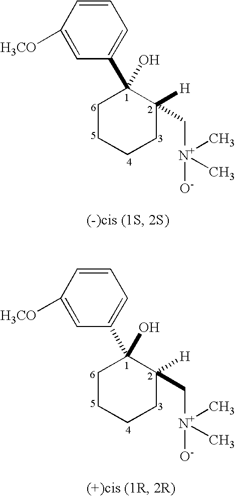

In other words, the compound of Formula III is the 50:50 mixture of the N-oxide of (+/−)cis-2-[(dimethylamino)methyl]-1-(3-methoxyphenyl) cyclohexanol.

In a further embodiment, the N-oxide is predominantly the (+)cis enantiomer, as set forth above.

In one embodiment, the substituted aminomethyl-phenyl-cyclohexane derivative suitable for use in the invention is represented by structural Formula IV:

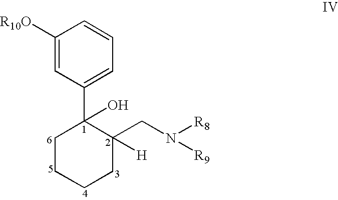

and enantiomers and mixtures thereof wherein:

R₈, R₉ and R₁₀ are independently hydrogen or an alkyl group;

or pharmaceutically acceptable salts, solvates or hydrates thereof.

In a particular embodiment, the compound of Formula IV is a mixture of the (+)cis and (−)cis enantiomers, wherein the C-1 and C-2 carbons of the cyclohexyl ring are (1R,2R) and (1S,2S), respectively, and the substituents on C-1 and C-2 are in the cis orientation.

In a specific embodiment, the mixture of the (+)cis and (−)cis enantiomers is a racemic mixture. That is, the compound of Formula IV is a 50:50 mixture of (+)cis and (−)cis enantiomers as shown below:

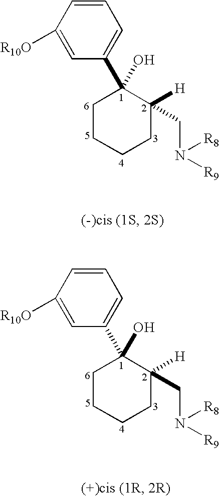

In a further embodiment, the compounds of Formula IV are predominantly the (+)cis enantiomer, as set forth above.

In a particular embodiment R₁₀ is hydrogen. In a further embodiment wherein R₁₀ is hydrogen, R₈ and R₉ are independently hydrogen or an alkyl group, for example, a methyl group. When R₁₀ is hydrogen and R₈ and R₉ are methyl groups, and Formula IV is the racemic mixture of the (+)cis and (−)cis enantiomers, the compound can be referred to as O-desmethyltramadol. The specific (+) and (−) enantiomers set forth above, can be referred to as (+)O-desmethyltramadol and (−)O-desmethyltramadol.

In yet another embodiment, R₁₀ is hydrogen, R₈ is hydrogen and R₉ is a methyl group. When R₁₀ is hydrogen, R8, is hydrogen and R9 is a methyl group, and Formula IV is the racemic mixture of the (+)cis and (−)cis enantiomers, the compound can be referred to as O-desmethyl-N-mono-desmethyl-tramadol. The specific (+)cis and (−)cis enantiomers as set forth above can be referred to as (+)O-desmethyl-N-mono-desmethyl-tramadol and (−)O-desmethyl-N-mono-desmethyl-tramadol.

In yet another embodiment, the substituted aminomethyl-phenyl-cyclohexane derivative is represented by structural Formula V:

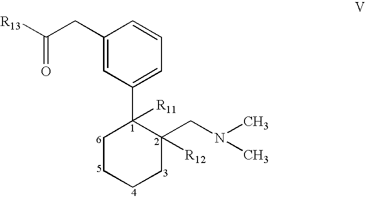

and enantiomers and mixtures thereof wherein:

R₁₁ is —OH;

R₁₂ is hydrogen or R₁₁ and R₁₂ together form a double bond;

R₁₃ is an aryl group selected from the group consisting of:

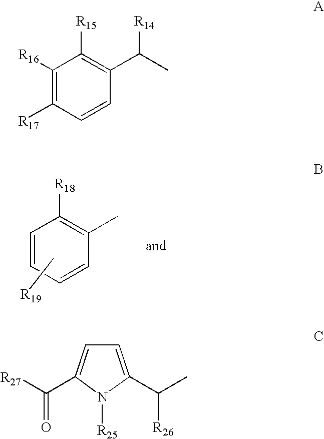

wherein:

R₁₄ is hydrogen or an alkyl group;

R₁₅ is hydrogen, —NH₂, —NHR₂₀ or —OR₂₀;

R₁₆ is hydrogen, —COR₂₀, —OR₂₀ or halogen;

R₁₇ is hydrogen, an alkyl group, —O-alkenyl, a phenyl group or R₁₆ and R₁₇ are —CH═CR₂₁—CR₂₂+CH—, forming an aromatic ring;

R₁₈ is hydrogen, —COR₂₃, —OR₂₄ or a halogen;

R₁₉ is hydrogen, halogen, an alkyl group, —O-alkyl, —NO₂ or an aryl group;

R₂₀ is a phenyl group optionally substituted by one or more of the following: halogen, —NO₂, an alkyl group, an alkenyl group, —OH or —NH₂;

R₂₁ and R₂₂ are independently hydrogen or —O-alkyl;

R₂₃ is a phenyl group optionally substituted by one or more of the following: halogen, —NO2, an alkyl group, and alkenyl group, —OH or —NH₂;

R₂₄ is hydrogen, —CO-alkyl (preferably methyl) or a phenyl group optionally substituted by one or more of the following: halogen, —NO₂, an alkyl group, and alkenyl group, —OH or —NH₂;

R₂₅ and R₂₆ are independently hydrogen, an alkyl group or form a —CH₂—CH₂-group;

R₂₇ is a phenyl group optionally substituted by one or more of the following: halogen, —NO₂, an alkyl group, an alkenyl group, —OH or —NH₂;

or pharmaceutically acceptable salts, solvates or hydrates thereof.

In a particular embodiment of Formula V, R₁₁ is —OH, R₁₂ is H and R₁₃ is:

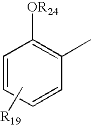

wherein:

R₂₄ is hydrogen or —COCH₃;

R₁₉ is halogen, an alkyl group, —O-alkyl or —NO₂.

It is preferred that when R₁₉ is —O-alkyl, the alkyl group is a methyl group.

It is preferred that when R₁₉ is an alkyl group, the alkyl group is substituted with one or more halogens. For example the substituted alkyl group is —CF₃.

Substituted aminomethyl-phenyl-cyclohexane derivatives in accordance with Formula V are further described in U.S. Pat. No. 6,455,585 and published PCT Application WO 01/49650, which are incorporated herein by reference.

5-HT₃ antagonists that may be employed as additional active agents in the present invention include, but are not limited to:

a. Ondansetron [1,2,3,9-tetrahydro-9-methyl-3-[(2-methyl-1H-imidazol-1-yl]methyl]-4H-carb azol-4-one (cf. Merck Index, twelfth edition, item 6979);

b. Granisetron [endo-1-methyl—N-(9-methyl-9-aza-bicyclo[3.3.1]non-3-yl)-1H-imidazole-3-carboxamide: (cf. Merck Index, twelfth edition, item 4557);

c. Dolasetron [1H-indole-3-carboxylic acid (2.alpha., 6.alpha., 8.alpha., 9.alpha.beta.)-octahydro-3-oxo-2,6methano-2H-quinolizin-8-yl ester] (cf. Merck Index, twelfth edition, item 3471);

d. Indol-3-yl-carboxylic acid-endo-8-methyl-8-aza-bicyclo[3,2,1]-oct-3-yl-ester, also known as tropisetron. (cf. Merck Index, twelfth edition, item 9914);

e. 4,5,6,7-tetrahydro-5-[(1-methyl-indol-3yl)carbonyl]benzimidazole (see also ramosetron, U.S. Pat. No. 5,344,927);

f. (+)-10-methyl-7-(5-methyl-1H-imidazol-4-ylmethyl)-6,7,8,9-tetrahydropyrido[1,2-a]indol-6-one (see also fabesetron, European Pat. No. 0 361 317);

g. [N-(1-ethyl-2-imidazolin-2-yl-methyl)-2-methoxy-4-amino-5-chlorobenzamide (see also lintopride-Chem.-Abstr.-No. 107429-63-0); and

h. 2,3,4,5-tetrahydro-5-methyl-2-[(5-methyl-1H-imidazol-4-yl)methyl]-1H-pyrido[4,3-b]indol-1-one (see also alosetron, European Pat. No. 0 306 323).

5-HT₄ agonists that may be employed as additional active agents in the present invention include, but are not limited to 2-piperazinylbenzothiazole and 2-piperazinylbenzoxazole derivatives as disclosed in Monge et al. (1994) J. Med. Chem. 37: 1320-1325.

Formulations

Formulations of the present invention may include, but are not limited to, continuous, as needed, short-term, rapid-offset, controlled release, sustained release, delayed release, and pulsatile release formulations.

Compositions of the invention comprise sodium channel modulators, particularly tetrodotoxin-resistant (TTX-R) sodium channel modulators and/or activity-dependent sodium channel modulators. TTX-R sodium channel modulators for use in the present invention include but are not limited to compounds that interact with Nav.1.8 and/or Na_{v.}1.9 channels. The compositions are administered in therapeutically effective amounts to a patient in need thereof for treating painful and non-painful lower urinary tract disorders in normal and spinal cord injured patients. It is recognized that the compositions may be administered by any means of administration as long as an effective amount for the treatment of painful and non-painful symptoms associated with lower urinary tract disorders is delivered.

Any of the active agents may be administered in the form of a salt, ester, amide, prodrug, active metabolite, derivative, or the like, provided that the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base using conventional methodology, and involves reaction with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are salts prepared with organic acids. Conversely, preparation of basic salts of acid moieties which may be present on an active agent are prepared in a similar manner using a pharmaceutically acceptable base such as sodium hydroxide, potassium hydroxide, ammonium hydroxide, calcium hydroxide, trimethylamine, or the like.

Preparation of esters involves functionalization of hydroxyl and/or carboxyl groups that may be present within the molecular structure of the drug. The esters are typically acyl-substituted derivatives of free alcohol groups, i.e., moieties that are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

Other salts, enantiomers, analogs, esters, amides, prodrugs, active metabolites, and derivatives of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

Pharmaceutical Compositions and Dosage Forms

Suitable compositions and dosage forms include tablets, capsules, caplets, pills, gel caps, troches, dispersions, suspensions, solutions, syrups, transdermal patches, gels, powders, magmas, lozenges, creams, pastes, plasters, lotions, discs, suppositories, liquid sprays for nasal or oral administration, dry powder or aerosolized formulations for inhalation, compositions and formulations for intravesical administration and the like. Further, those of ordinary skill in the art can readily deduce that suitable formulations involving these compositions and dosage forms, including those formulations as described elsewhere herein.

Oral Dosage Forms

Oral dosage forms include tablets, capsules, caplets, solutions, suspensions and/or syrups, and may also comprise a plurality of granules, beads, powders or pellets that may or may not be encapsulated. Such dosage forms are prepared using conventional methods known to those in the field of pharmaceutical formulation and described in the pertinent texts, e.g., in Remington: The Science and Practice of Pharmacy, supra). Tablets and capsules represent the most convenient oral dosage forms, in which case solid pharmaceutical carriers are employed.

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

In addition to the active agent(s), then, tablets prepared for oral administration using the method of the invention will generally contain other materials such as binders, diluents, lubricants, disintegrants, fillers, stabilizers, surfactants, preservatives, coloring agents, flavoring agents and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum. Diluents are typically necessary to increase bulk so that a practical size tablet is ultimately provided. Suitable diluents include dicalcium phosphate, calcium sulfate, lactose, cellulose, kaolin, mannitol, sodium chloride, dry starch and powdered sugar. Lubricants are used to facilitate tablet manufacture; examples of suitable lubricants include, for example, vegetable oils such as peanut oil, cottonseed oil, sesame oil, olive oil, corn oil, and oil of theobroma, glycerin, magnesium stearate, calcium stearate, and stearic acid. Stearates, if present, preferably represent at no more than approximately 2 wt. % of the drug-containing core. Disintegrants are used to facilitate disintegration of the tablet, and are generally starches, clays, celluloses, algins, gums or crosslinked polymers. Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Stabilizers are used to inhibit or retard drug decomposition reactions that include, by way of example, oxidative reactions. Surfactants may be anionic, cationic, amphoteric or nonionic surface active agents.

The dosage form may also be a capsule, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. (See, for e.g., Remington: The Science and Practice of Pharmacy, supra), which describes materials and methods for preparing encapsulated pharmaceuticals. If the active agent-containing composition is present within the capsule in liquid form, a liquid carrier is necessary to dissolve the active agent(s). The carrier must be compatible with the capsule material and all components of the pharmaceutical composition, and must be suitable for ingestion.

Solid dosage forms, whether tablets, capsules, caplets, or particulates, may, if desired, be coated so as to provide for delayed release. Dosage forms with delayed release coatings may be manufactured using standard coating procedures and equipment. Such procedures are known to those skilled in the art and described in the pertinent texts (See, for e.g., Remington: The Science and Practice of Pharmacy, supra). Generally, after preparation of the solid dosage form, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained release dosage forms provide for drug release over an extended time period, and may or may not be delayed release. Generally, as will be appreciated by those of ordinary skill in the art, sustained release dosage forms are formulated by dispersing a drug within a matrix of a gradually bioerodible (hydrolyzable) material such as an insoluble plastic, a hydrophilic polymer, or a fatty compound, or by coating a solid, drug-containing dosage form with such a material. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

Transmucosal Compositions and Dosage Forms

Although the present compositions may be administered orally, other modes of administration are suitable as well. For example, transmucosal administration may be advantageously employed. Transmucosal administration is carried out using any type of formulation or dosage unit suitable for application to mucosal tissue. For example, the selected active agent may be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

Preferred buccal dosage forms will typically comprise a therapeutically effective amount of an active agent and a bioerodible(hydrolyzable)polymeric carrier that may also serve to adhere the dosage form to the buccal mucosa. The buccal dosage unit is fabricated so as to erode over a predetermined time period, wherein drug delivery is provided essentially throughout. The time period is typically in the range of from about 1 hour to about 72 hours. Preferred buccal delivery preferably occurs over a time period of from about 2 hours to about 24 hours. Buccal drug delivery for short term use should preferably occur over a time period of from about 2 hours to about 8 hours, more preferably over a time period of from about 3 hours to about 4 hours. As needed buccal drug delivery preferably will occur over a time period of from about 1 hour to about 12 hours, more preferably from about 2 hours to about 8 hours, most preferably from about 3 hours to about 6 hours. Sustained buccal drug delivery will preferably occur over a time period of from about 6 hours to about 72 hours, more preferably from about 12 hours to about 48 hours, most preferably from about 24 hours to about 48 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver.

The “therapeutically effective amount” of the active agent in the buccal dosage unit will of course depend on the potency of the agent and the intended dosage, which, in turn, is dependent on the particular individual undergoing treatment, the specific indication, and the like. The buccal dosage unit will generally contain from about 1.0 wt. % to about 60 wt. % active agent, preferably on the order of from about 1 wt. % to about 30 wt. % active agent. With regard to the bioerodible (hydrolyzable) polymeric carrier, it will be appreciated that virtually any such carrier can be used, so long as the desired drug release profile is not compromised, and the carrier is compatible with the sodium channel modulator, particularly tetrodotoxin-resistant (TTX-R) sodium channel modulator and/or activity-dependent sodium channel modulator, to be administered and any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and co, e.g., those known as “carbomers” (Carbopol®, which may be obtained from B. F. Goodrich, is one such polymer). Other suitable polymers include, but are not limited to: hydrolyzed polyvinylalcohol; polyethylene oxides (e.g., Sentry Polyox® water soluble resins, available from Union Carbide); polyacrylates (e.g., Gantrez®, which may be obtained from GAF); vinyl polymers and copolymers; polyvinylpyrrolidone; dextran; guar gum; pectins; starches; and cellulosic polymers such as hydroxypropyl methylcellulose, (e.g., Methocel®, which may be obtained from the Dow Chemical Company), hydroxypropyl cellulose (e.g., Klucel®, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components may also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone® XL, which may be obtained from GAF), cross-linked carboxylic methylcelluloses, such as croscarmelose (e.g., Ac-di-sol®, which may be obtained from FMC), alginic acid, and sodium carboxymethyl starches (e.g., Explotab®, which may be obtained from Edward Medell Co., Inc.), methylcellulose, agar bentonite and alginic acid. Suitable diluents are those which are generally useful in pharmaceutical formulations prepared using compression techniques, e.g., dicalcium phosphate dihydrate (e.g., Di-Tab®, which may be obtained from Stauffer), sugars that have been processed by cocrystallization with dextrin (e.g., co-crystallized sucrose and dextrin such as Di-Pak®, which may be obtained from Amstar), calcium phosphate, cellulose, kaolin, mannitol, sodium chloride, dry starch, powdered sugar and the like. Binders, if used, are those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Sublingual and lingual dosage forms include tablets, creams, ointments, lozenges, pastes, and any other solid dosage form where the active ingredient is admixed into a disintegrable matrix. The tablet, cream, ointment or paste for sublingual or lingual delivery comprises a therapeutically effective amount of the selected active agent and one or more conventional nontoxic carriers suitable for sublingual or lingual drug administration. The sublingual and lingual dosage forms of the present invention can be manufactured using conventional processes. The sublingual and lingual dosage units are fabricated to disintegrate rapidly. The time period for complete disintegration of the dosage unit is typically in the range of from about 10 seconds to about 30 minutes, and optimally is less than 5 minutes.

Other components may also be incorporated into the sublingual and lingual dosage forms described herein. The additional components include, but are not limited to binders, disintegrants, wetting agents, lubricants, and the like. Examples of binders that may be used include water, ethanol, polyvinylpyrrolidone; starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like. Particularly preferred lubricants are stearates and polyethylene glycol. Additional components that may be incorporated into sublingual and lingual dosage forms are known, or will be apparent, to those skilled in this art (See, e.g., Remington: The Science and Practice of Pharmacy, supra).

For transurethral administration, the formulation comprises a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials, with polyethylene glycol and derivatives thereof particularly preferred.

Depending on the particular active agent administered, it may be desirable to incorporate a transurethral permeation enhancer in the urethral dosage form. Examples of suitable transurethral permeation enhancers include dimethylsulfoxide (“DMSO”), dimethyl formamide (“DMF”), N,N-dimethylacetamide (“DMA”), decylmethylsulfoxide (“C₁₀MSO”), polyethylene glycol monolaurate (“PEGML”), glycerol monolaurate, lecithin, the 1-substituted, azacycloheptan-2-ones, particularly 1-n-dodecylcyclazacycloheptan-2-one (available under the trademark Azone® from Nelson Research & Development Co., Irvine, Calif.), SEPA® (available from Macrochem Co., Lexington, Mass.), surfactants as discussed above, including, for example, Tergitol®, Nonoxynol-9® and TWEEN-80®, and lower alkanols such as ethanol.

Transurethral drug administration, as explained in U.S. Pat. Nos. 5,242,391, 5,474,535, 5,686,093 and 5,773,020, can be carried out in a number of different ways using a variety of urethral dosage forms. For example, the drug can be introduced into the urethra from a flexible tube, squeeze bottle, pump or aerosol spray. The drug may also be contained in coatings, pellets or'suppositories that are absorbed, melted or bioeroded in the urethra. In certain embodiments, the drug is included in a coating on the exterior surface of a penile insert. It is preferred, although not essential, that the drug be delivered from at least about 3 cm into the urethra, and preferably from at least about 7 cm into the urethra. Generally, delivery from at least about 3 cm to about 8 cm into the urethra will provide effective results in conjunction with the present method.

Urethral suppository formulations containing PEG or a PEG derivative may be conveniently formulated using conventional techniques, e.g., compression molding, heat molding or the like, as will be appreciated by those skilled in the art and as described in the pertinent literature and pharmaceutical texts. (See, e.g., Remington: The Science and Practice of Pharmacy, supra), which discloses typical methods of preparing pharmaceutical compositions in the form of urethral suppositories. The PEG or PEG derivative preferably has a molecular weight in the range of from about 200 to about 2,500 g/mol, more preferably in the range of from about 1,000 to about 2,000 g/mol. Suitable polyethylene glycol derivatives include polyethylene glycol fatty acid esters, for example, polyethylene glycol monostearate, polyethylene glycol sorbitan esters, e.g., polysorbates, and the like. Depending on the particular active agent, it may also be preferred that urethral suppositories contain one or more solubilizing agents effective to increase the solubility of the active agent in the PEG or other transurethral vehicle.

It may be desirable to deliver the active agent in a urethral dosage form that provides for controlled or sustained release of the agent. In such a case, the dosage form comprises a biocompatible, biodegradable material, typically a biodegradable polymer. Examples of such polymers include polyesters, polyalkylcyanoacrylates, polyorthoesters, polyanhydrides, albumin, gelatin and starch. As explained, for example, in PCT Publication No. WO 96/40054, these and other polymers can be used to provide biodegradable microparticles that enable controlled and sustained drug release, in turn minimizing the required dosing frequency.

The urethral dosage form will preferably comprise a suppository that is on the order of from about 2 to about 20 mm in length, preferably from about 5 to about 10 mm in length, and less than about 5 mm in width, preferably less than about 2 mm in width. The weight of the suppository will typically be in the range of from about 1 mg to about 100 mg, preferably in the range of from about 1 mg to about 50 mg. However, it will be appreciated by those skilled in the art that the size of the suppository can and will vary, depending on the potency of the drug, the nature of the formulation, and other factors.

Transurethral drug delivery may involve an “active” delivery mechanism such as iontophoresis, electroporation or phonophoresis. Devices and methods for delivering drugs in this way are well known in the art. lontophoretically assisted drug-delivery is, for example, described in PCT Publication No. WO 96/40054, cited above. Briefly, the active agent is driven through the urethral wall by means of an electric current passed from an external electrode to a second electrode contained within or affixed to a urethral probe.

Preferred transrectal dosage forms include rectal suppositories, creams, ointments, and liquid formulations (enemas). The suppository, cream, ointment or liquid formulation for transrectal delivery comprises a therapeutically effective amount of the selected phosphodiesterase inhibitor and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration is preferably in the range of from about 10 minutes to about 6 hours, and optimally is less than about 3 hours.

Other components may also be incorporated into the transrectal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite. Preferred vaginal or perivaginal dosage forms include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit can be fabricated to disintegrate rapidly or over a period of several hours. The time period for complete disintegration is preferably in the range of from about 10 minutes to about 6 hours, and optimally is less than about 3 hours.

Other components may also be incorporated into the vaginal or perivaginal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

The active agents may also be administered intranasally or by inhalation. Compositions for intranasal administration are generally liquid formulations for administration as a spray or in the form of drops, although powder formulations for intranasal administration, e.g., insufflations, are also known, as are nasal gels, creams, pastes or ointments. For liquid formulations, the active agent can be formulated into a solution, e.g., water or isotonic saline, buffered or unbuffered, or as a suspension. Preferably, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, simply by way of example, phosphate buffers. Furthermore, various devices are available in the art for the generation of drops, droplets and sprays, including droppers, squeeze bottles, and manually and electrically powered intranasal pump dispensers. Active agent containing intranasal carriers may also include nasal gels, creams, pastes or ointments with a viscosity of, e.g., from about 10 to about 6500 cps, or greater, depending on the desired sustained contact with the nasal mucosal surfaces. Such carrier viscous formulations may be based upon, simply by way of example, alkylcelluloses and/or other biocompatible carriers of high viscosity well known to the art (see e.g., Remington: The Science and Practice of Pharmacy, supra). Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation.

Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation may take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation may also comprise dry powder formulations, particularly insufflations in which the powder has an average particle size of from about 0.1 μm to about 50 μm, preferably from about 1 μm to about 25 μm.

Topical Formulations

Topical formulations may be in any form suitable for application to the body surface, and may comprise, for example, an ointment, cream, gel, lotion, solution, paste or the like, and/or may be prepared so as to contain liposomes, micelles, and/or microspheres. Preferred topical formulations herein are ointments, creams and gels.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, supra, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight (See, e.g., Remington: The Science and Practice of Pharmacy, supra).

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal“phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels-are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the topical formulations. For example, solubilizers may be used to solubilize certain active agents. For those drugs having an unusually low rate of permeation through the skin or mucosal tissue, it may be desirable to include a permeation enhancer in the formulation; suitable enhancers are as described elsewhere herein.

Transdermal Administration

The compounds of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present, the backing is preferably made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well known in the art and include, for example, those enhancers listed above in transmucosal compositions.

Parenteral Administration

Parenteral administration, if used, is generally characterized by injection, including intramuscular, intraperitoneal, intravenous (IV) and subcutaneous injection. Injectable formulations can be prepared in conventional forms, either as liquid solutions or suspensions; solid forms suitable for solution or suspension in liquid prior to injection, or as emulsions. Preferably, sterile injectable suspensions are formulated according to techniques known in the art using suitable dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. A more recently revised approach for parenteral administration involves use of a slow release or sustained release system (See, e.g., U.S. Pat. No. 3,710,795).

Intravesical Administration

Intravesical administration, if used, is generally characterized by administration directly into the bladder and may include methods as described elsewhere herein. Other methods of intravesical administration may include those described in U.S. Pat. Nos. 6,207,180 and 6,039,967, as well as other methods that are known to one of skill in the art.

Intrathecal Administration

Intrathecal administration, if used, is generally characterized by administration directly into the intrathecal space (where fluid flows around the spinal cord).

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication can then be administered directly to cells in the spinal cord involved in conveying sensory and motor signals associated with treat painful and non-painful lower urinary tract disorders.

Another system available from Medtronic that is commonly utilized for intrathecal administration is the is the fully implantable, programmable SynchroMed® Infusion System. The SynchroMed® Infusion System has two parts that are both placed in the body during a surgical procedure: the catheter and the pump. The catheter is a small, soft tube. One end is connected to the catheter port of the pump, and the other end is placed in the intrathecal space. The pump is a round metal device about one inch (2.5 cm) thick, three inches (8.5 cm) in diameter, and weighs about six ounces (205 g) that stores and releases prescribed amounts of medication directly into the intrathecal space. It is made of titanium, a lightweight, medical-grade metal. The reservoir is the space inside the pump that holds the medication. The fill port is a raised center portion of the pump through which the pump is refilled. The doctor or a nurse inserts a needle through the patient's skin and through the fill port to fill the pump. Some pumps have a side catheter access port that allows the doctor to inject other medications or sterile solutions directly into the catheter, bypassing the pump.

The SynchroMed® pump automatically delivers a controlled amount of medication through the catheter to the intrathecal space around the spinal cord, where it is most effective. The exact dosage, rate and timing prescribed by the doctor are entered in the pump using a programmer, an external computer-like device that controls the pump's memory. Information about the patient's prescription is stored in the pump's memory. The doctor can easily review this information by using the programmer. The programmer communicates with the pump by radio signals that allow the doctor to tell how the pump is operating at any given time. The doctor also can use the programmer to change your medication dosage.

Methods of intrathecal administration may include those described above available from Medtronic, as well as other methods that are known to one of skill in the art.

Additional Dosage Formulations and Drug Delivery Systems

As compared with traditional drug delivery approaches, some controlled release technologies rely upon the modification of both macromolecules and synthetic small molecules to allow them to be actively instead of passively absorbed into the body. For example, XenoPort Inc. utilizes technology that takes existing molecules and re-engineers them to create new chemical entities (unique molecules) that have improved pharmacologic properties to either: 1) lengthen the short half-life of a drug; 2) overcome poor absorption; and/or 3) deal with poor drug distribution to target tissues. Techniques to lengthen the short half-life of a drug include the use of prodrugs with slow cleavage rates to release drugs over time or that engage transporters in small and large intestines to allow the use of oral sustained delivery systems, as well as drugs that engage active transport systems. Examples of such controlled release formulations, tablets, dosage forms, and drug delivery systems, and that are suitable for use with the present invention, are described in the following published US and PCT patent applications assigned to Xenoport Inc.: US20030158254; US20030158089; US20030017964; US2003130246; WO02100172; WO02100392; WO02100347; WO02100344; WO0242414; WO0228881; WO0228882; WO0244324; WO0232376; WO0228883; and WO0228411. Some other controlled release technologies rely upon methods that promote or enhance gastric retention, such as those developed by Depomed Inc. Because many drugs are best absorbed in the stomach and upper portions of the small intestine, Depomed has developed tablets that swell in the stomach during the postprandial or fed mode so that they are treated like undigested food. These tablets therefore sit safely and neutrally in the stomach for 6, 8, or more hours and deliver drug at a desired rate and time to upper gastrointestinal sites. Specific technologies in this area include: 1) tablets that slowly erode in gastric fluids to deliver drugs at almost a constant rate (particularly useful for highly insoluble drugs); 2) bi-layer tablets that combine drugs with different characteristics into a single table (such as a highly insoluble drug in an erosion layer and a soluble drug in a diffusion layer for sustained release of both); and 3) combination tablets that can either deliver drugs simultaneously or in sequence over a desired period of time (including an initial burst of a fast acting drug followed by slow and sustained delivery of another drug). Examples of such controlled release formulations that are suitable for use with the present invention and that rely upon gastric retention during the postprandial or fed mode, include tablets, dosage forms, and drug delivery systems in the following US patents assigned to Depomed Inc.: U.S. Pat. No. 6,488,962; U.S. Pat. No. 6,451,808; U.S. Pat. No. 6,340,475; U.S. Pat. No. 5,972,389; U.S. Pat. No. 5,582,837; and U.S. Pat. No. 5,007,790. Examples of such controlled release formulations that are suitable for use with the present invention and that rely upon gastric retention during the postprandial or fed mode, include tablets, dosage forms, and drug delivery systems in the following published US and PCT patent applications assigned to Depomed Inc.: US20030147952; US20030104062; US20030104053; US20030104052; US20030091630; US20030044466; US20030039688; US20020051820; WO0335040; WO0335039; WO0156544; WO0132217; WO9855107; WO9747285; and WO9318755.

Other controlled release systems include those developed by ALZA Corporation based upon: 1) osmotic technology for oral delivery; 2) transdermal delivery via patches; 3) liposomal delivery via intravenous injection; 4) osmotic technology for long-term delivery via implants; and 5) depot technology designed to deliver agents for periods of days to a month. ALZA oral delivery systems include those that employ osmosis to provide precise, controlled drug delivery for up to 24 hours for both poorly soluble and highly soluble drugs, as well as those that deliver high drug doses meeting high drug loading requirements. ALZA controlled transdermal delivery systems provide drug delivery through intact skin for as long as one week with a single application to improve drug absorption and deliver constant amounts of drug into the bloodstream over time. ALZA liposomal delivery systems involve lipid nanoparticles that evade recognition by the immune system because of their unique polyethylene glycol (PEG) coating, allowing the precise delivery of drugs to disease-specific areas of the body. ALZA also has developed osmotically driven systems to enable the continuous delivery of small drugs, peptides, proteins, DNA and other bioactive macromolecules for up to one year for systemic or tissue-specific therapy. Finally, ALZA depot injection therapy is designed to deliver biopharmaceutical agents and small molecules for periods of days to a month using a nonaqueous polymer solution for the stabilization of macromolecules and a unique delivery profile.

Examples of controlled release formulations, tablets, dosage forms, and drug delivery systems that are suitable for use with the present invention are described in the following US patents assigned to ALZA Corporation: U.S. Pat. No. 4,367,741; U.S. Pat. No. 4,402,695; U.S. Pat. No. 4,418,038; U.S. Pat. No. 4,434,153; U.S. Pat. No. 4,439,199; U.S. P 4,450,198; U.S. Pat. No. 4,455,142; U.S. Pat. No. 4,455,144; U.S. Pat. No. 4,484,923; U.S. Pat. No. 4,486,193; U.S. Pat. No. 4,489,197; U.S. Pat. No. 4,511,353; U.S. Pat. No. 4,519,801; U.S. Pat. No. 4,526,578; U.S. Pat. No. 4,526,933; U.S. Pat. No. 4,534,757; U.S. Pat. No. 4,553,973; U.S. Pat. No. 4,559,222; U.S. Pat. No. 4,564,364; U.S. Pat. No. 4,578,075; U.S. Pat. No. 4,588,580; U.S. Pat. No. 4,610,686; U.S. Pat. No. 4,618,487; U.S. Pat. No. 4,627,851; U.S. Pat. No. 4,629,449; U.S. Pat. No. 4,642,233; U.S. Pat. No. 4,649,043; U.S. Pat. No. 4,650,484; U.S. Pat. No. 4,659,558; U.S. Pat. No. 4,661,105; U.S. Pat. No. 4,662,880; U.S. Pat. No. 4,675,174; U.S. Pat. No. 4,681,583; U.S. Pat. No. 4,684,524; U.S. Pat. No. 4,692,336; U.S. Pat. No. 4,693,895; U.S. Pat. No. 4,704,119; U.S. Pat. No. 4,705,515; U.S. Pat. No. 4,717,566; U.S. Pat. No. 4,721,613; U.S. Pat. No. 4,723,957; U.S. Pat. No. 4,725,272; U.S. Pat. No. 4,728,498; U.S. Pat. No. 4,743,248; U.S. Pat. No. 4,747,847; U.S. Pat. No. 4,751,071; U.S. Pat. No. 4,753,802; U.S. Pat. No. 4,755,180; U.S. Pat. No. 4,756,314; U.S. Pat. No. 4,764,380; U.S. Pat. No. 4,773,907; U.S. Pat. No. 4,777,049; U.S. Pat. No. 4,781,924; U.S. Pat. No. 4,786,503; U.S. Pat. No. 4,788,062; U.S. Pat. No. 4,810,502; U.S. Pat. No. 4,812,313; U.S. Pat. No. 4,816,258; U.S. Pat. No. 4,824,675; U.S. Pat. No. 4,834,979; U.S. Pat. No. 4,837,027; U.S. Pat. No. 4,842,867; U.S. Pat. No. 4,846,826; U.S. Pat. No. 4,847,093; U.S. Pat. No. 4,849,226; U.S. Pat. No. 4,851,229; U.S. Pat. No. 4,851,231; U.S. Pat. No. 4,851,232; U.S. Pat. No. 4,853,229; U.S. Pat. No. 4,857,330; U.S. Pat. No. 4,859,470; U.S. Pat. No. 4,863,456; U.S. Pat. No. 4,863,744; U.S. Pat. No. 4,865,598; U.S. Pat. No. 4,867,969; U.S. Pat. No. 4,871,548; U.S. Pat. No. 4,872,873; U.S. Pat. No. 4,874,388; U.S. Pat. No. 4,876,093; U.S. Pat. No. 4,892,778; U.S. Pat. No. 4,902,514; U.S. Pat. No. 4,904,474; U.S. Pat. No. 4,913,903; U.S. Pat. No. 4,915,949; U.S. Pat. No. 4,915,952; U.S. Pat. No. 4,917,895; U.S. Pat. No. 4,931,285; U.S. Pat. No. 4,946,685; U.S. Pat. No. 4,948,592; U.S. Pat. No. 4,954,344; U.S. Pat. No. 4,957,494; U.S. Pat. No. 4,960,416; U.S. Pat. No. 4,961,931; U.S. Pat. No. 4,961,932; U.S. Pat. No. 4,963,141; U.S. Pat. No. 4,966,769; U.S. Pat. No. 4,971,790; U.S. Pat. No. 4,976,966; U.S. Pat. No. 4,986,987; U.S. Pat. No. 5,006,346; U.S. Pat. No. 5,017,381; U.S. Pat. No. 5,019,397; U.S. Pat. No. 5,023,076; U.S. Pat. No. 5,023,088; U.S. Pat. No. 5,024,842; U.S. Pat. No. 5,028,434; U.S. Pat. No. 5,030,454; U.S. Pat. No. 5,071,656; U.S. Pat. No. 5,077,054; U.S. Pat. No. 5,082,668; U.S. Pat. No. 5,104,390; U.S. Pat. No. 5,110,597; U.S. Pat. No. 5,122,128; U.S. Pat. No. 5,125,894; U.S. Pat. No. 5,141,750; U.S. Pat. No. 5,141,752; U.S. Pat. No. 5,156,850; U.S. Pat. No. 5,160,743; U.S. Pat. No. 5,160,744; U.S. Pat. No. 5,169,382; U.S. Pat. No. 5,171,576; U.S. Pat. No. 5,176,665; U.S. Pat. No. 5,185,158; U.S. Pat. No. 5,190,765; U.S. Pat. No. 5,198,223; U.S. Pat. No. 5,198,229; U.S. Pat. No. 5,200,195; U.S. Pat. No. 5,200,196; U.S. Pat. No. 5,204,116; U.S. Pat. No. 5,208,037; U.S. Pat. No. 5,209,746; U.S. Pat. No. 5,221,254; U.S. Pat. No. 5,221,278; U.S. Pat. No. 5,229,133; U.S. Pat. No. 5,232,438; U.S. Pat. No. 5,232,705; U.S. Pat. No. 5,236,689; U.S. Pat. No. 5,236,714; U.S. Pat. No. 5,240,713; U.S. Pat. No. 5,246,710; U.S. Pat. No. 5,246,711; U.S. Pat. No. 5,252,338; U.S. Pat. No. 5,254,349; U.S. Pat. No. 5,266,332; U.S. Pat. No. 5,273,752; U.S. Pat. No. 5,284,660; U.S. Pat. No. 5,286,491; U.S. Pat. No. 5,308,348; U.S. Pat. No. 5,318,558; U.S. Pat. No. 5,320,850; U.S. Pat. No. 5,322,502; U.S. Pat. No. 5,326,571; U.S. Pat. No. 5,330,762; U.S. Pat. No. 5,338,550; U.S. Pat. No. 5,340,590; U.S. Pat. No. 5,342,623; U.S. Pat. No. 5,344,656; U.S. Pat. No. 5,348,746; U.S. Pat. No. 5,358,721; U.S. Pat. No. 5,364,630; U.S. Pat. No. 5,376,377; U.S. Pat. No. 5,391,381; U.S. Pat. No. 5,402,777; U.S. Pat. No. 5,403,275; U.S. Pat. No. 5,411,740; U.S. Pat. No. 5,417,675; U.S. Pat. No. 5,417,676; U.S. Pat. No. 5,417,682; U.S. Pat. No. 5,423,739; U.S. Pat. No. 5,424,289; U.S. Pat. No. 5,431,919; U.S. Pat. No. 5,443,442; U.S. Pat. No. 5,443,459; U.S. Pat. No. 5,443,461; U.S. Pat. No. 5,456,679; U.S. Pat. No. 5,460,826; U.S. Pat. No. 5,462,741; U.S. Pat. No. 5,462,745; U.S. Pat. No. 5,489,281; U.S. Pat. No. 5,499,979; U.S. Pat. No. 5,500,222; U.S. Pat. No. 5,512,293; U.S. Pat. No. 5,512,299; U.S. Pat. No. 5,529,787; U.S. Pat. No. 5,531,736; U.S. Pat. No. 5,532,003; U.S. Pat. No. 5,533,971; U.S. Pat. No. 5,534,263; U.S. Pat. No. 5,540,912; U.S. Pat. No. 5,543,156; U.S. Pat. No. 5,571,525; U.S. Pat. No. 5,573,503; U.S. Pat. No. 5,591,124; U.S. Pat. No. 5,593,695; U.S. Pat. No. 5,595,759; U.S. Pat. No. 5,603,954; U.S. Pat. No. 5,607,696; U.S. Pat. No. 5,609,885; U.S. Pat. No. 5,614,211; U.S. Pat. No. 5,614,578; U.S. Pat. No. 5,620,705; U.S. Pat. No. 5,620,708; U.S. Pat. No. 5,622,530; U.S. Pat. No. 5,622,944; U.S. Pat. No. 5,633,011; U.S. Pat. No. 5,639,477; U.S. Pat. No. 5,660,861; U.S. Pat. No. 5,667,804; U.S. Pat. No. 5,667,805; U.S. Pat. No. 5,674,895; U.S. Pat. No. 5,688,518; U.S. Pat. No. 5,698,224; U.S. Pat. No. 5,702,725; U.S. Pat. No. 5,702,727; U.S. Pat. No. 5,707,663; U.S. Pat. No. 5,713,852; U.S. Pat. No. 5,718,700; U.S. Pat. No. 5,736,580; U.S. Pat. No. 5,770,227; U.S. Pat. No. 5,780,058; U.S. Pat. No. 5,783,213; U.S. Pat. No. 5,785,994; U.S. Pat. No. 5,795,591; U.S. Pat. No. 5,811,465; U.S. Pat. No. 5,817,624; U.S. Pat. No. 5,824,340; U.S. Pat. No. 5,830,501; U.S. Pat. No. 5,830,502; U.S. Pat. No. 5,840,754; U.S. Pat. No. 5,858,407; U.S. Pat. No. 5,861,439; U.S. Pat. No. 5,863,558; U.S. Pat. No. 5,876,750; U.S. Pat. No. 5,883,135; U.S. Pat. No. 5,897,878; U.S. Pat. No. 5,904,934; U.S. Pat. No. 5,904,935; U.S. Pat. No. 5,906,832; U.S. Pat. No. 5,912,268; U.S. Pat. No. 5,914,131; U.S. Pat. No. 5,916,582; U.S. Pat. No. 5,932,547; U.S. Pat. No. 5,938,654; U.S. Pat. No. 5,941,844; U.S. Pat. No. 5,955,103; U.S. Pat. No. 5,972,369; U.S. Pat. No. 5,972,370; U.S. Pat. No. 5,972,379; U.S. Pat. No. 5,980,943; U.S. Pat. No. 5,981,489; U.S. Pat. No. 5,983,130; U.S. Pat. No. 5,989,590; U.S. Pat. No. 5,995,869; U.S. Pat. No. 5,997,902; U.S. Pat. No. 6,001,390; U.S. Pat. No. 6,004,309; U.S. Pat. No. 6,004,578; U.S. Pat. No. 6,008,187; U.S. Pat. No. 6,020,000; U.S. Pat. No. 6,034,101; U.S. Pat. No. 6,036,973; U.S. Pat. No. 6,039,977; U.S. Pat. No. 6,057,374; U.S. Pat. No. 6,066,619; U.S. Pat. No. 6,068,850; U.S. Pat. No. 6,077,538; U.S. Pat. No. 6,083,190; U.S. Pat. No. 6,096,339; U.S. Pat. No. 6,106,845; U.S. Pat. No. 6,110,499; U.S. Pat. No. 6,120,798; U.S. Pat. No. 6,120,803; U.S. Pat. No. 6,124,261; U.S. Pat. No. 6,130,200; U.S. Pat. No. 6,146,662; U.S. Pat. No. 6,153,678; U.S. Pat. No. 6,174,547; U.S. Pat. No. 6,183,466; U.S. Pat. No. 6,203,817; U.S. Pat. No. 6,210,712; U.S. Pat. No. 6,210,713; U.S. Pat. No. 6,224,907; U.S. Pat. No. 6,235,712; U.S. Pat. No. 6,245,357; U.S. Pat. No. 6,262,115; U.S. Pat. No. 6,264,990; U.S. Pat. No. 6,267,984; U.S. Pat. No. 6,287,598; U.S. Pat. No. 6,289,241; U.S. Pat. No. 6,331,311; U.S. Pat. No. 6,333,050; U.S. Pat. No. 6,342,249; U.S. Pat. No. 6,346,270; U.S. Pat. No. 6,365,183; U.S. Pat. No. 6,368,626; U.S. Pat. No. 6,387,403; U.S. Pat. No. 6,419,952; U.S. Pat. No. 6,440,457; U.S. Pat. No. 6,468,961; U.S. Pat. No. 6,491,683; U.S. Pat. No. 6,512,010; U.S. Pat. No. 6,514,530; U.S. Pat. No. 6534089; U.S. Pat. No. 6,544,252; U.S. Pat. No. 6,548,083; U.S. Pat. No. 6,551,613; U.S. Pat. No. 6,572,879; and U.S. Pat. No. 6,596,314.

Other examples of controlled release formulations, tablets, dosage forms, and drug delivery systems that are suitable for use with the present invention are described in the following published US patent application and PCT applications assigned to ALZA Corporation: US20010051183; WO0004886; WO0013663; WO0013674; WO0025753; WO0025790; WO0035419; WO0038650; WO0040218; WO0045790; WO0066126; WO0074650; WO0119337; WO0119352; WO0121211; WO0137815; WO0141742; WO0143721; WO0156543; WO3041684; WO03041685; WO03041757; WO03045352; WO03051341; WO03053400; WO03053401; WO9000416; WO9004965; WO9113613; WO9116884; WO9204011; WO9211843; WO9212692; WO9213521; WO9217239; WO9218102; WO9300071; WO9305843; WO9306819; WO9314813; WO9319739; WO9320127; WO9320134; WO9407562; WO9408572; WO9416699; WO9421262; WO9427587; WO9427589; WO9503823; WO9519174; WO9529665; WO9600065; WO9613248; WO9625922; WO9637202; WO9640049; WO9640050; WO9640139; WO9640364; WO9640365; WO9703634; WO9800158; WO9802169; WO9814168; WO9816250; WO9817315; WO9827962; WO9827963; WO9843611; WO9907342; WO9912526; WO9912527; WO9918159; WO9929297; WO9929348; WO9932096; WO9932153; WO9948494; WO9956730; WO9958115; and WO9962496.

Andrx Corporation has also developed drug delivery technology suitable for use in the present invention that includes: 1) a pelletized pulsatile delivery system (“PPDS”); 2) a single composition osmotic tablet system (“SCOT”); 3) a solubility modulating hydrogel system (“SMHS”); 4) a delayed pulsatile hydrogel system (“DPHS”); 5) a stabilized pellet delivery system (“SPDS”); 6) a granulated modulating hydrogel system (“GMHS”); 7) a pelletized tablet system (“PELTAB”); 8) a porous tablet system (“PORTAB”); and 9) a stabilized tablet delivery system (“STDS”). PPDS uses pellets that are coated with specific polymers and agents to control the release rate of the microencapsulated drug and is designed for use with drugs that require a pulsed release. SCOT utilizes various osmotic modulating agents as well as polymer coatings to provide a zero-order drug release. SMHS utilizes a hydrogel-based dosage system that avoids the “initial burst effect” commonly observed with other sustained-release hydrogel formulations and that provides for sustained release without the need to use special coatings or structures that add to the cost of manufacturing. DPHS is designed for use with hydrogel matrix products characterized by an initial zero-order drug release followed by a rapid release that is achieved by the blending of selected hydrogel polymers to achieve a delayed pulse. SPDS incorporates a pellet core of drug and protective polymer outer layer, and is designed specifically for unstable drugs, while GMHS incorporates hydrogel and binding polymers with the drug and forms granules that are pressed into tablet form. PELTAB provides controlled release by using a water insoluble polymer to coat discrete drug crystals or pellets to enable them to resist the action of fluids in the gastrointestinal tract, and these coated pellets are then compressed into tablets. PORTAB provides controlled release by incorporating an osmotic core with a continuous polymer coating and a water soluble component that expands the core and creates microporous channels through which drug is released. Finally, STDS includes a dual layer coating technique that avoids the need to use a coating layer to separate the enteric coating layer from the omeprazole core.

Examples of controlled release formulations, tablets, dosage forms, and drug delivery systems that are suitable for use with the present invention are described in the following US patents assigned to Andrx Corporation: U.S. Pat. No. 5,397,574; U.S. Pat. No. 5,419,917; U.S. Pat. No. 5,458,887; U.S. Pat. No. 5,458,888; U.S. Pat. No. 5,472,708; U.S. Pat. No. 5,508,040; U.S. Pat. No. 5,558,879; U.S. Pat. No. 5,567,441; U.S. Pat. No. 5,654,005; U.S. Pat. No. 5,728,402; U.S. Pat. No. 5,736,159; U.S. Pat. No.5,830,503; U.S. Pat. No. 5,834,023; U.S. Pat. No. 5,837,379; U.S. Pat. No. 5,916,595; U.S. Pat. No. 5,922,352; U.S. Pat. No. 6,099,859; U.S. Pat. No. 6,099,862; U.S. Pat. No. 6,103,263; U.S. Pat. No. 6,106,862; U.S. Pat. No. 6,156,342; U.S. Pat. No. 6,177,102; U.S. Pat. No. 6,197,347; U.S. Pat. No. 6,210,716; U.S. Pat. No. 6,238,703; U.S. Pat. No. 6,270,805; U.S. Pat. No. 6,284,275; U.S. Pat. No. 6,485,748; U.S. Pat. No. 6,495,162; U.S. Pat. No. 6,524,620; U.S. Pat. No. 6,544,556; U.S. Pat. No. 6,589,553; U.S. Pat. No. 6,602,522; and U.S. Pat. No. 6,610,326.

Examples of controlled release formulations, tablets, dosage forms, and drug delivery systems that are suitable for use with the present invention are described in the following published US and PCT patent applications assigned to Andrx Corporation: US20010024659; US20020115718; US20020156066; WO0004883; WO0009091; WO0012097; WO0027370; WO0050010; WO0132161; WO0134123; WO0236077; WO0236100; WO02062299; WO02062824; WO02065991; WO02069888; WO02074285; WO03000177; WO9521607; WO9629992; WO9633700; WO9640080; WO9748386; WO9833488; WO9833489; WO9930692; WO9947125; and WO9961005.

Some other examples of drug delivery approaches focus on non-oral drug delivery, providing parenteral, transmucosal, and topical delivery of proteins, peptides, and small molecules. For example, the Atrigel® drug delivery system marketed by Atrix Laboratories Inc. comprises biodegradable polymers, similar to those used in biodegradable sutures, dissolved in biocompatible carriers. These pharmaceuticals may be blended into a liquid delivery system at the time of manufacturing or, depending upon the product, may be added later by a physician at the time of use. Injection of the liquid product subcutaneously or intramuscularly through a small gauge needle, or placement into accessible tissue sites through a cannula, causes displacement of the carrier with water in the tissue fluids, and a subsequent precipitate to form from the polymer into a solid film or implant. The drug encapsulated within the implant is then released in a controlled manner as the polymer matrix biodegrades over a period ranging from days to months. Examples of such drug delivery systems include Atrix's Eligard®, Atridox®/Doxirobe®, Atrisorb® FreeFlow™/Atrisorb®-D FreeFlow, bone growth products, and others as described in the following published US and PCT patent applications assigned to Atrix Laboratories Inc.: US RE37950; U.S. Pat. No. 6,630,155; U.S. Pat. No. 6,566,144; U.S. Pat. No. 6,610,252; U.S. Pat. No. 6,565,874; U.S. Pat. No. 6,528,080; U.S. Pat. No. 6,461,631; U.S. Pat. No. 6,395,293; U.S. Pat. No. 6,261,583; U.S. Pat. No. 6,143,314; U.S. Pat. No. 6,120,789; U.S. Pat. No. 6,071,530; U.S. Pat. No. 5,990,194; U.S. Pat. No. 5,945,115; U.S. Pat. No. 5,888,533; U.S. Pat. No. 5,792,469; U.S. Pat. No. 5,780,044; U.S. Pat. No. 5,759,563; U.S. Pat. No. 5,744,153; U.S. Pat. No. 5,739,176; U.S. Pat. No. 5,736,152; U.S. Pat. No. 5,733,950; U.S. Pat. No. 5,702,716; U.S. Pat. No. 5,681,873; U.S. Pat. No. 5,660,849; U.S. Pat. No. 5,599,552; U.S. Pat. No. 5,487,897; U.S. Pat. No. 5,368,859; U.S. Pat. No. 5,340,849; U.S. Pat. No. 5,324,519; U.S. Pat. No. 5,278,202; U.S. Pat. No. 5,278,201; US20020114737, US20030195489; US20030133964;U.S. Pat. No. 20010042317; US20020090398; US20020001608; and US2001042317.

Atrix Laboratories Inc. also markets technology for the non-oral transmucosal delivery of drugs over a time period from minutes to hours. For example, Atrix's BEMA™ (Bioerodible Muco-Adhesive Disc) drug delivery system comprises pre-formed bioerodible discs for local or systemic delivery. Examples of such drug delivery systems include those as described in U.S. Pat. No. 6,245,345.

Other drug delivery systems marketed by Atrix Laboratories Inc. focus on topical drug delivery. For example, SMP™ (Solvent Particle System) allows the topical delivery of highly water-insoluble drugs. This product allows for a controlled amount of a dissolved drug to permeate the epidermal layer of the skin by combining the dissolved drug with a microparticle suspension of the drug. The SMP™ system works in stages whereby: 1) the product is applied to the skin surface; 2) the product near follicles concentrates at the skin pore; 3) the drug readily partitions into skin oils; and 4) the drug diffuses throughout the area. By contrast, MCA® (Mucocutaneous Absorption System) is a water-resistant topical gel providing sustained drug delivery. MCA® forms a tenacious film for either wet or dry surfaces where: 1) the product is applied to the skin or mucosal surface; 2) the product forms a tenacious moisture-resistant film; and 3) the adhered film provides sustained release of drug for a period from hours to days. Yet another product, BCP™ (Biocompatible Polymer System) provides a non-cytotoxic gel or liquid that is applied as a protective film for wound healing. Examples of these systems include Orajel®-Ultra Mouth Sore Medicine as well as those as described in the following published US patents and applications assigned to Atrix Laboratories Inc.: U.S. Pat. No. 6,537,565; U.S. Pat. No. 6,432,415; U.S. Pat. No. 6,355,657; U.S. Pat. No. 5,962,006; U.S. Pat. No.5,725,491; U.S. Pat. No. 5,722,950; U.S. Pat. No. 5,717,030; U.S. Pat. No. 5,707,647; U.S. Pat. No. 5,632,727; and US20010033853.

Dosage and Administration

The concentration of the active agent in any of the aforementioned dosage forms and compositions can vary a great deal, and will depend on a variety of factors, including the type of composition or dosage form, the corresponding mode of administration, the nature and activity of the specific active agent, and the intended drug release profile. Preferred dosage forms contain a unit dose of active agent, i.e., a single therapeutically effective dose. For creams, ointments, etc., a “unit dose” requires an active agent concentration that provides a unit dose in a specified quantity of the formulation to be applied. The unit dose of any particular active agent will depend, of course, on the active agent and on the mode of administration. For a sodium channel modulator, particularly a TTX-R sodium channel modulator and/or activity-dependent sodium channel modulator, the unit dose for oral administration will be in the range of from about 1 mg to about 10,000 mg, typically in the range of from about 100 mg to about 5,000 mg; for local administration, suitable unit doses may be lower. Alternatively, for a sodium channel modulator, particularly a TTX-R sodium channel modulator and/or activity-dependent sodium channel modulator, the unit dose for oral administration will be greater than about 1 mg, about 5 mg, about 10 mg, about 20 mg, about 30 mg, about 40 mg, about 50 mg, about 100 mg, about 200 mg, about 300 mg, about 400 mg, about 500 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, or about 9,500 mg. Those of ordinary skill in the art of pharmaceutical formulation can readily deduce suitable unit doses for sodium channel modulators, particularly TTX-R sodium channel modulators and/or activity-dependent sodium channel modulators, as well as suitable unit doses for other types of agents that may be incorporated into a dosage form of the invention.

For sodium channel modulators, particularly TTX-R sodium channel modulators and/or activity-dependent sodium channel modulators, the unit dose for transmucosal, topical, transdermal, intravesical, and parenteral administration will be in the range of from about 1 ng to about 10,000 mg, typically in the range of from about 100 ng to about 5,000 mg. Alternatively, for sodium channel modulators, particularly TTX-R sodium channel modulators and/or activity-dependent sodium channel modulators, the unit dose for transmucosal, topical, transdermal, intravesical, and parenteral administration will be greater than about 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, about 500 μg, about 1 mg, about 5 mg, about 10 mg, about 20 mg, about 30 mg, about 40 mg, about 50 mg, about 100 mg, about 200 mg, about 300 mg, about 400 mg, about 500 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, or about 9,500 mg. Those of ordinary skill in the art of pharmaceutical formulation can readily deduce suitable unit doses for sodium channel modulators, particularly TTX-R sodium channel modulators and/or activity-dependent sodium channel modulator, as well as suitable unit doses for other types of agents that may be incorporated into a dosage form of the invention.

For sodium channel modulators, particularly TTX-R sodium channel modulators and/or activity-dependent sodium channel modulators, the unit dose for intrathecal administration will be in the range of from about 1 fg to about 1 mg, typically in the range of from about 100 fg to about 1 ng. Alternatively, for sodium channel modulators, particularly TTX-R sodium channel modulators and/or activity-dependent sodium channel modulators, the unit dose for intrathecal administration will be greater than about 1 fg, about 5 fg, about 10 fg, about 20 fg, about 30 fg, about 40 fg, about 50 fg, about 100 fg, about 200 fg, about 300 fg, about 400 fg, about 500 fg, about 1 pg, about 5 pg, about 10 pg, about 20 pg, about 30 pg, about 40 pg, about 50 pg, about 100 pg, about 200 pg, about 300 pg, about 400 pg, about 500 pg, about 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, or about 500 μg. Those of ordinary skill in the art of pharmaceutical formulation can readily deduce suitable unit doses for sodium channel modulators, particularly TTX-R sodium channel modulators and/or activity-dependent sodium channel modulators, as well as suitable unit doses for other types of agents that may be incorporated into a dosage form of the invention.

A therapeutically effective amount of a particular active agent administered to a given individual will, of course, be dependent on a number of factors, including the concentration of the specific active agent, composition or dosage form, the selected mode of administration, the age and general condition of the individual being treated, the severity of the individual's condition, and other factors known to the prescribing physician.

In a preferred embodiment, drug administration is on an as-needed basis, and does not involve chronic drug administration. With an immediate release dosage form, as-needed administration may involve drug administration immediately prior to commencement of an activity wherein suppression of the symptoms of overactive bladder would be desirable, but will generally be in the range of from about 0 minutes to about 10 hours prior to such an activity, preferably in the range of from about 0 minutes to about 5 hours prior to such an activity, most preferably in the range of from about 0 minutes to about 3 hours prior to such an activity. With a sustained release dosage form, a single dose can provide therapeutic efficacy over an extended time period in the range of from about 1 hour to about 72 hours, typically in the range of from about 8 hours to about 48 hours, depending on the formulation. That is, the release period may be varied by the selection and relative quantity of particular sustained release polymers. If necessary, however, drug administration may be carried out within the context of an ongoing dosage regimen, i.e., on a weekly basis, twice weekly, daily, etc.

Packaged Kits

In another embodiment, a packaged kit is provided that contains the pharmaceutical formulation to be administered, i.e., a pharmaceutical formulation containing a therapeutically effective amount of a selected active agent for the treatment of painful and non-painful lower urinary tract disorders, such as painful and non-painful overactive bladder, a container, preferably sealed, for housing the formulation during storage and prior to use, and instructions for carrying out drug administration in a manner effective to treat painful and non-painful lower urinary tract disorders, such as painful and non-painful overactive bladder. The instructions will typically be written instructions on a package insert and/or on a label. Depending on the type of formulation and the intended mode of administration, the kit may also include a device for administering the formulation. The formulation may be any suitable formulation as described herein. For example, the formulation may be an oral dosage form containing a unit dosage of a selected active agent. The kit may contain multiple formulations of different dosages of the same agent. The kit may also contain multiple formulations of different active agents.

Insurance Claims

In general, the processing of an insurance claim for the coverage of a given medical treatment or drug therapy involves notification of the insurance company, or any other entity, that has issued the insurance policy against which the claim is being filed, that the medical treatment or drug therapy will be performed. A determination is then made as to whether the medical treatment or drug therapy that will be performed is covered under the terms of the policy. If covered, the claim is then processed, which can include payment, reimbursement, or application against a deductable.

The present invention encompasses a method for processing an insurance claim under an insurance policy for a sodium channel modulator, particularly a TTX-R sodium channel modulator and/or activity-dependent sodium channel modulator, or pharmaceutically acceptable salts, esters, amides, prodrugs, or active metabolites thereof used in the treatment of lower urinary tract disorders. This method comprises: 1) receiving notification that treatment of a lower urinary tract disorder using said sodium channel modulator, particularly a TTX-R sodium channel modulator and/or activity-dependent sodium channel modulator, or pharmaceutically acceptable salts, esters, amides, prodrugs or active metabolites thereof will be performed or receiving notification of a prescription for said sodium channel modulator to treat lower urinary tract disorders; 2) determining whether said treatment using said sodium channel modulator, particularly a TTX-R sodium channel modulator and/or activity-dependent sodium channel modulator, or pharmaceutically acceptable salts, esters, amides, prodrugs or active metabolites is covered under said insurance policy; and 3) processing said claim for treatment using said sodium channel modulator, particularly a TTX-R sodium channel modulator and/or activity-dependent sodium channel modulator or pharmaceutically acceptable salts, esters, amides, prodrugs, or active metabolites thereof, including payment, reimbursement, or application against a deductable.

The present invention also encompasses the method for processing an insurance claim described above, wherein a sodium channel modulator, particularly a TTX-R sodium channel modulator and/or activity-dependent sodium channel modulator and a secondary agent are used in the treatment of lower urinary tract disorders. Secondary agents can include an antispasmodic, a tricyclic antidepressant, duloxetine, venlafaxine, a monoamine reuptake inhibitor, a spasmolytic, an anticholinergic, gabapentin, pregabalin, a substituted aminomethyl-phenyl-cyclohexane derivative, a 5-HT₃ antagonist, a 5-HT₄ antagonist, a β3 adrenergic agonist, a neurokinin receptor antagonist, a bradykinin receptor antagonist, a nitric oxide donor, or pharmaceutically acceptable salts, esters, amides, prodrugs, or active metabolites thereof. Futhermore, the method for processing an insurance claim according to the present invention encompasses wherein said sodium channel modulator, particularly a TTX-R sodium channel modulator and/or activity-dependent sodium channel modulator and said secondary agent, or pharmaceutically acceptable salts, esters, amides, prodrugs, or active metabolites thereof, are administered sequentially, concurrently in the same composition, or concurrently in different compositions. The method for processing an insurance claim according to the present invention also encompasses the processing of claims for a sodium channel modulator, particularly a TTX-R sodium channel modulator and/or activity-dependent sodium channel modulator and one of the secondary agents described above, or pharmaceutically acceptable salts, esters, amides, prodrugs, or active metabolites thereof, when either has been prescribed separately or concurrently for the treatment of lower urinary tract disorders.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended embodiments. Although specific terms are employed herein, they are used in a generic and descriptive sense only and not for purposes of limitation.

All patents, patent applications, and publications mentioned herein are hereby incorporated by reference in their entireties.

### EXAMPLES

Methods for Treating Painful and Non-Painful Lower Urinary Tract Disorders By Administering Sodium Channel Modulators

The invention will be further described in the following examples, which do not limit the scope of the invention described in the claims. The following examples illustrate the effects of administration of sodium channel modulators on well-accepted models for urinary tract disorders. It is expected that these results will demonstrate the efficacy of sodium channel modulators for treatment of painful and non-painful lower urinary tract disorders.

These methods include the use of a well accepted model for urinary tract disorders involving the bladder using intravesically administered acetic acid as described in Sasaki et al. (2002) J. Urol. 168: 1259-64. These methods also include the use of a well accepted model for urinary tract disorders involving examination of sodium channel currents recorded from bladder sensory neurons as described in Yoshimura & de Groat (1999) J. Neurosci. 19: 4644-4653.

### Example 1

### Dilute Acetic Acid Model

Objective and Rationale

The objective of the current study was to determine the effect of TTX-R sodium channel modulators or use dependent sodium channel modulators on the ability to reverse the reduction in bladder capacity seen following continuous infusion of dilute acetic acid, a commonly used model of lower urinary tract disorders including overactive bladder.

Materials and Methods

Animal Preparation: Female rats (250-275 g BW) were anesthetized with urethane (1.2 g/kg) and a saline-filled catheter (PE-50) was inserted into either the jugular vein for intravenous (i.v.; saline vehicle) or the proximal duodenum for intraduodenal (i.d.; distilled water or 10% Tween 80 in saline as vehicle) drug administration. Via a midline lower abdominal incision, a flared-tipped PE 50 catheter was inserted into the bladder dome for bladder filling and pressure recording and secured by ligation. The abdominal cavity was moistened with saline and closed by covering with a thin plastic sheet in order to maintain access to the bladder for emptying purposes. Fine silver or stainless steel wire electrodes were inserted into the external urethral sphincter (EUS) percutaneously for electromyography (EMG).

Experimental Design: Saline was continuously infused at a rate of 0.055 ml/min via the bladder filling catheter for ≧60 minutes to obtain a baseline of lower urinary tract activity (continuous cystometry; CMG). Following the control period, a 0.25% acetic acid solution in saline was infused into the bladder at the same flow rate to induce bladder irritation. Following 30 minutes of AA infusion, 3 vehicle injections were made at 20 minute intervals to determine vehicle effects, if any. Subsequently, increasing doses (2-5) of Na⁺ channel blocking compound were administered intravenously or intraduodenaly at half-log order increments at 30 or 60 minute intervals in order to construct a cumulative dose-response relationship. At the end of the control saline cystometry period, the third vehicle, and 20-50 minutes following each subsequent treatment, the infusion pump was stopped, the bladder was emptied by fluid withdrawal via the infusion catheter and a single filling cystometrogram was performed at the same flow rate in order to determine changes in bladder capacity caused by the irritation protocol and subsequent intravenous or intraduodenal drug administration.

Data Analysis

Data were analyzed by non-parametric ANOVA for repeated measures (Friedman Test) with Dunn's Multiple Comparison test. All comparisons were made from the last vehicle measurement (AA/Veh 3) or the lowest dose of drug. P<0.050 was considered significant.

Results and Conclusions

Intraduodenal ambroxol (n=5; 30-300 mg/kg), ralfinamide (n=7; 3-30 mg/kg), carbamazepine (n=8; 10-100 mg/kg), topiramate (n=7; 10-100 mg/kg), sipatrigine (n=7; 10-100 mg/kg), losigamone (n=4; 10-300 mg/kg), mexilitine (n=4; 10-30 mg/kg) and intravenous lidocoaine (n=5, 0.3-10 mg/kg) resulted in dose-dependent, statistically significant increases in bladder capacity, as measured by filling cystometry in rats during continuous irritation (See Table 1). By contrast, neither intraduodenal vinpocetine (n=6; 3-100 mg/kg) nor intravenous tolperisone (n=4; 3-10 mg/kg) demonstrated statistically significant effects on bladder capacity as measured by filling cystometry in rats during continuous irritation (See Table 1).

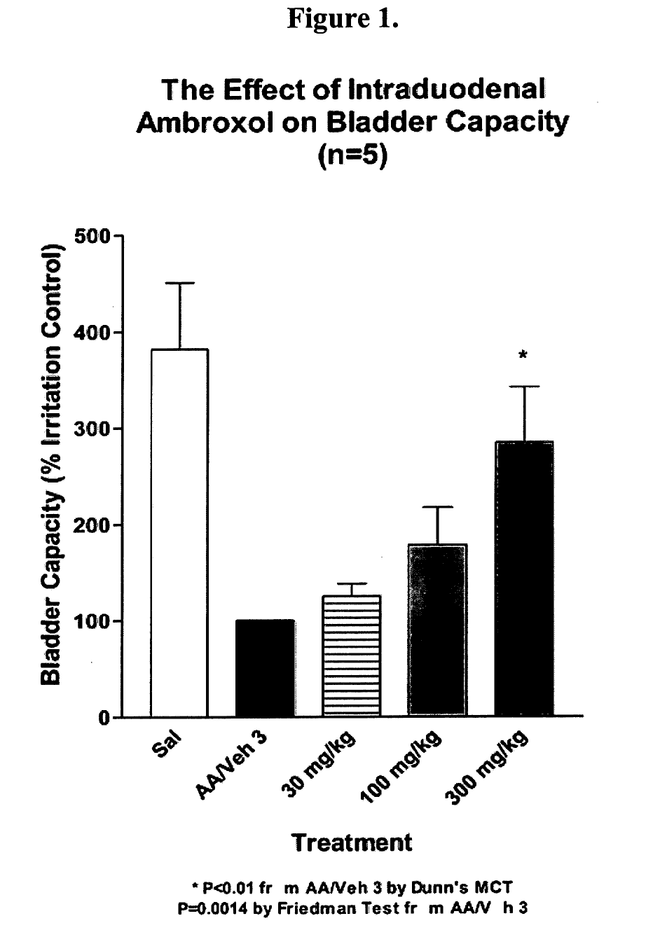

For Ambroxol, there was a dose-dependent and statistically significant reversal in the acetic acid-induced reduction of bladder capacity (FIG. 1; P=0.0014 by ANOVA). Post-test analysis revealed a statistically significant reversal of bladder capacity reduction at the 300 mg/kg dose (P<0.01).

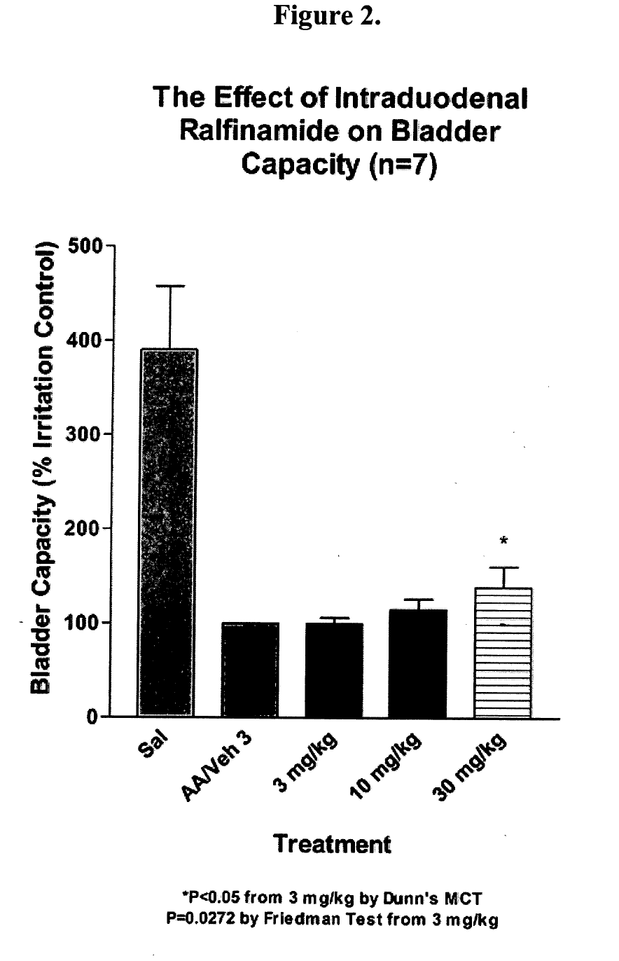

For Ralfinamide, there was a dose-dependent and statistically significant reversal in the acetic acid-induced reduction of bladder capacity (FIG. 2; P=0.0272 by ANOVA). Post-test analysis revealed a statistically significant reversal of bladder capacity reduction at the 30 mg/kg dose (P<0.05).

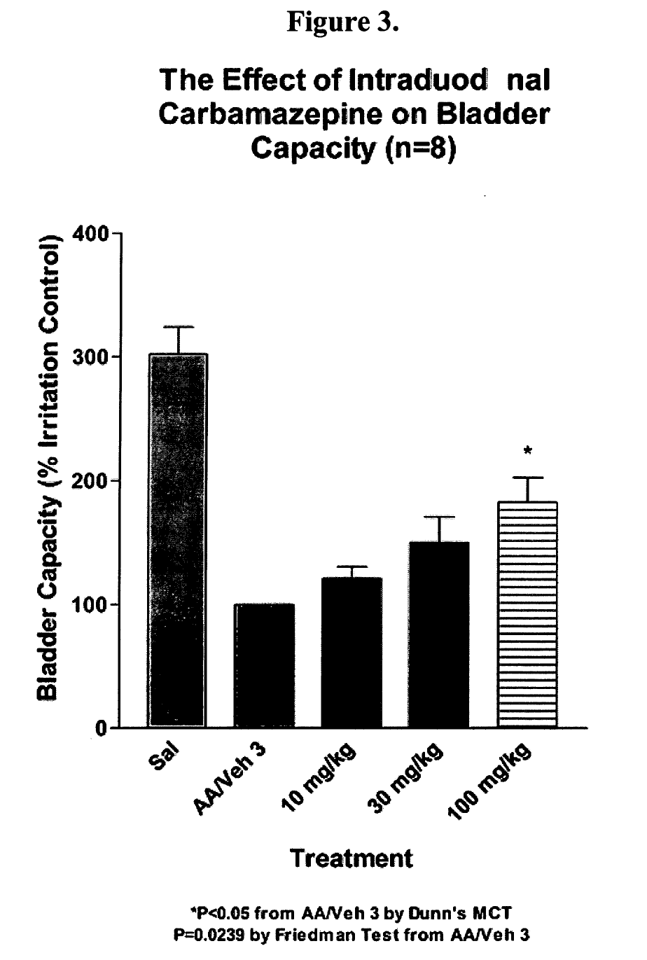

For Carbamazepine, there was a dose-dependent and statistically significant reversal in the acetic acid-induced reduction of bladder capacity (FIG. 3; P=0.0239 by ANOVA). Post-test analysis revealed a statistically significant reversal of bladder capacity reduction at the 100 mg/kg dose (P<0.05).

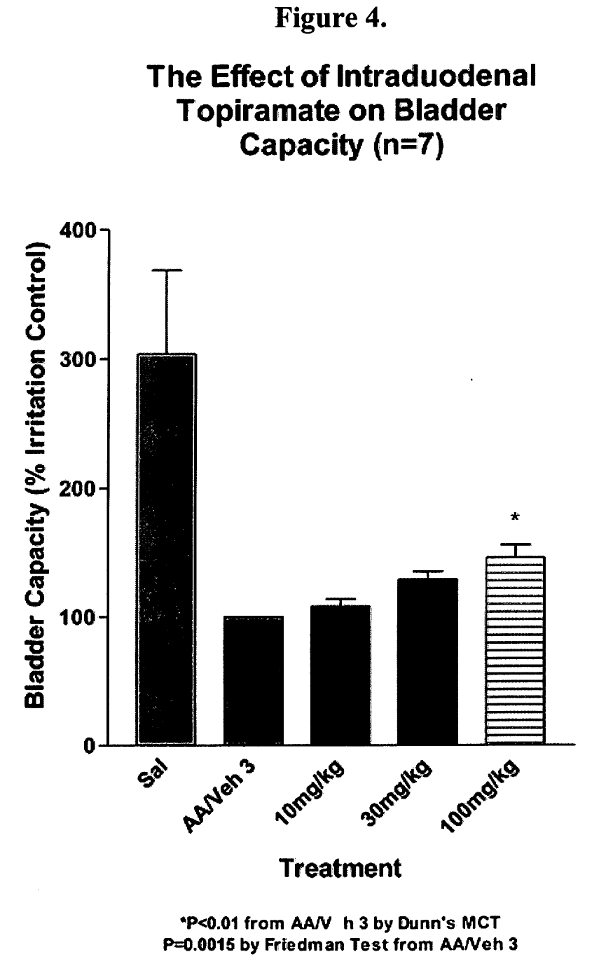

For Topiramate, there was a dose-dependent and statistically significant reversal in the acetic acid-induced reduction of bladder capacity (FIG. 4; P=0.0015 by ANOVA). Post-test analysis revealed a statistically significant reversal of bladder capacity reduction at the 100 mg/kg dose (P<0.01).

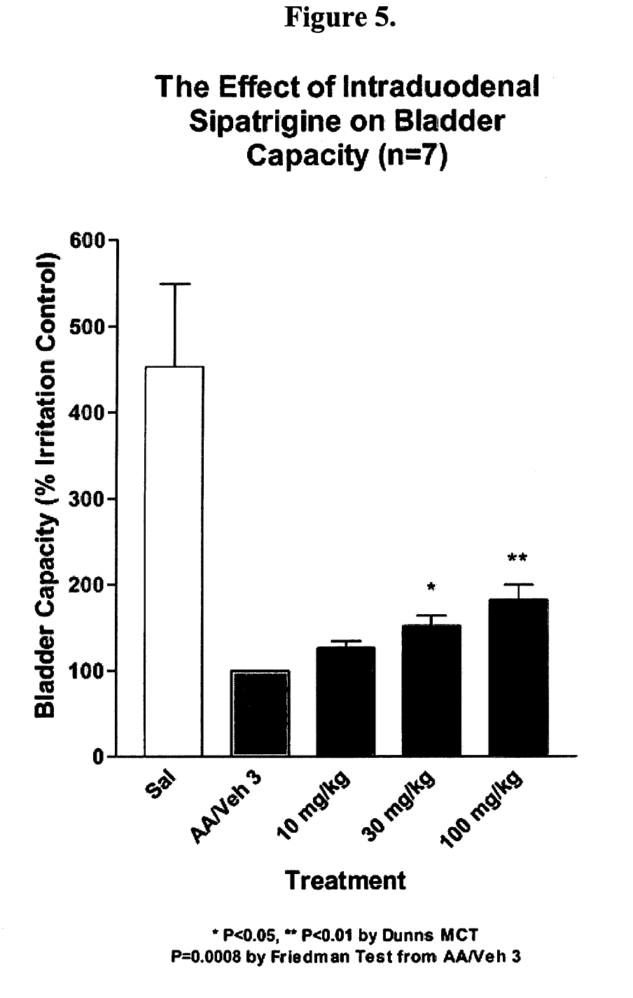

For Sipatrigine, there was a dose-dependent and statistically significant reversal in the acetic acid-induced reduction of bladder capacity (FIG. 5; P=0.0008 by ANOVA). Post-test analysis revealed statistically significant reversal of bladder capacity reduction at the 30 mg/kg dose (P<0.05) and the 100 mg/kg dose (P<0.01).

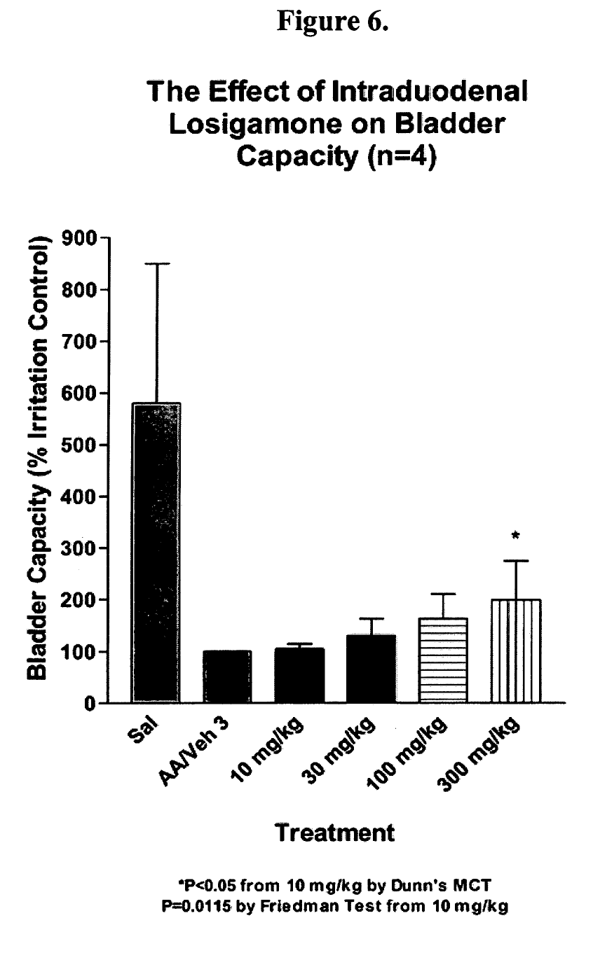

For Losigamone, there was a dose-dependent and statistically significant reversal in the acetic acid-induced reduction of bladder capacity (FIG. 6; P=0.0115 by ANOVA). Post-test analysis revealed a statistically significant reversal of bladder capacity reduction at the 300 mg/kg dose (P<0.05).

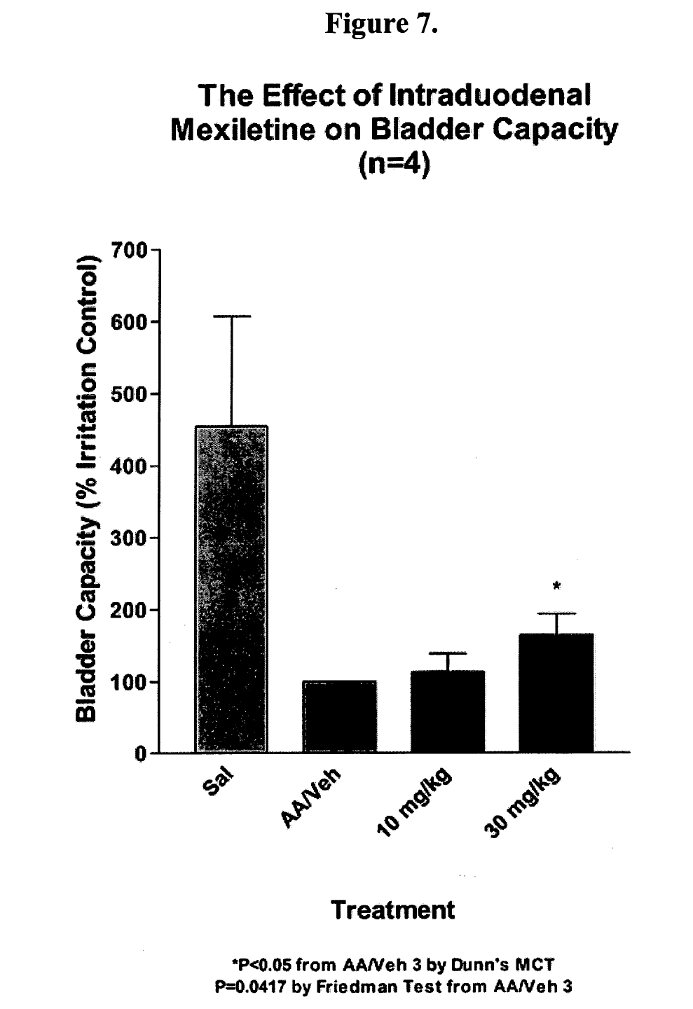

For Mexiletine, there was a dose-dependent and statistically significant reversal in the acetic acid-induced reduction of bladder capacity (FIG. 7; P=0.0417 by ANOVA). Post-test analysis revealed a statistically significant reversal of bladder capacity reduction at the 30 mg/kg dose (P<0.05).

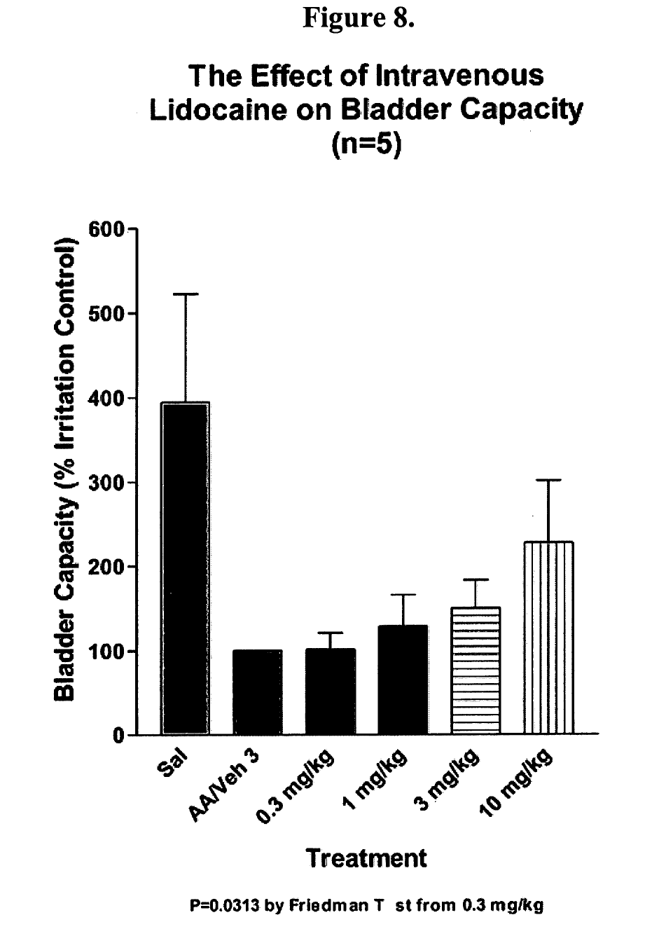

For Lidocaine, there was a dose-dependent and statistically significant reversal in the acetic acid-induced reduction of bladder capacity (FIG. 8; P=0.0313 by ANOVA).

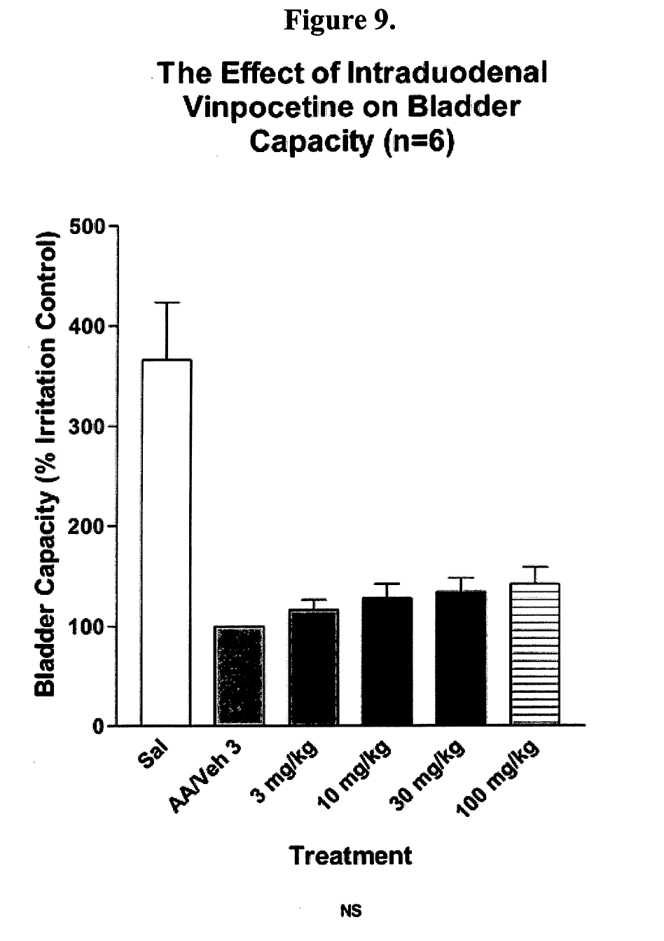

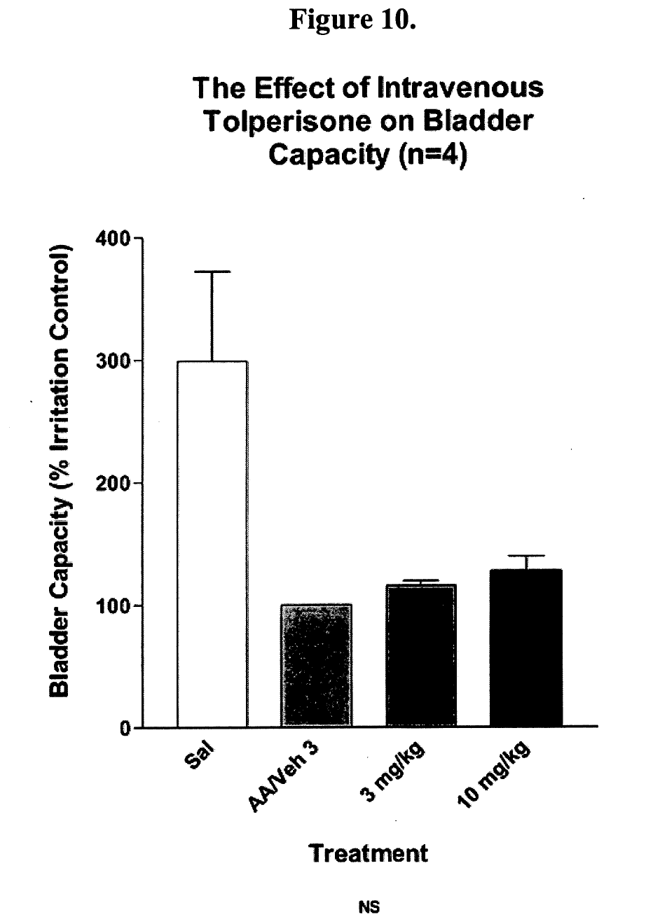

Neither Vinpocetine (FIG. 9) nor intravenous Tolperisone (FIG. 10) demonstrated statistically significant effects on bladder capacity as measured by filling cystometry in rats during continuous irritation.

The ability of agents primarily identified as sodium channel modulators to produce a dramatic reversal in acetic acid irritation-induced reduction in bladder capacity strongly indicates efficacy in mammalian forms of painful and nonpainful lower urinary tract disorders including overactive bladder.

TABLE 1

| Compound | | Route/ | Significant | Significant |
|---|---|---|---|---|
| Tested | N | Vehicle | Dose-Response | Post-test |
| Ambroxol | 5 | i.d./tween | + | + |
| Ralfinamide | 7 | i.d./dH₂O | + | + |
| Carbamazepine | 8 | i.d./tween | + | + |
| Topiramate | 7 | i.d./tween | + | + |
| Sipatrigine | 7 | i.d./tween | + | + |
| Losigamone | 4 | i.d./tween | + | + |
| Mexiletine | 4 | i.d./tween | + | + |
| Lidocaine | 5 | i.v./saline | + | − |
| Vinpocetine | 6 | i.d./tween | − | − |
| Tolperisone | 4 | i.v./saline | − | − |

### Example 2

### Bladder Sensory Neuron Sodium Channel Current Model

Objective and Rationale

The objective of the current study was to determine the effect of TTX-R sodium channel modulators or use dependent sodium channel modulators on the ability to modulate sodium currents in bladder primary afferent neurons, a commonly used model of lower urinary tract disorders including overactive bladder.

Methods

Labeling of bladder afferent neurons: Adult female Sprague-Dawley rats (150-300 g) were deeply anesthetized with pentobarbital anesthesia and placed on isoflurane maintenance anesthesia. A ventral midline incision was made through the abdominal skin and musculature, exposing the urinary bladder. Five injections of the fluorescent dye Di-I (5 μl each of 25 mg/ml Di-I in DMSO) or Fast Blue (4% w/v) were made into the bladder smooth muscle wall to label primary afferent fibers innervating the bladder. The area was rinsed with sterile saline to eliminate nonspecific spread of dye, and the incision was closed. Rats recovered for 5-12 days to allow for transport of fluorescent dye from distal terminals to the cell somata of dorsal root ganglion (DRG) neurons. Labeled neurons were identified in vitro using fluorescence optics.

Neuronal cultures: Di-I injected rats were euthanized with pentobarbital anesthesia. Lumbar (L6) and sacral (S₁) DRG were dissected from the vertebral column and placed in Dulbecco's modified Eagles medium (DMEM) containing 0.3% collagenase B for 60 min at 37° C. The cell solution was exchanged for a 0.25% trypsin in calcium/magnesium-free Dulbecco's phosphate-buffered saline solution, and further digested for 30 min at 37° C. Following a wash in fresh DMEM, ganglia were dissociated by a series of triturations using fire-polished Pasteur pipettes. DRG cells were plated on polylysine-treated glass coverslips. Cells were plated at a density of 0.5 DRG per coverslip in 1 ml DMEM supplemented with 10% FBS, NGF, and 100 U/ml penicillin/streptomycin. All experimental procedures involving rats were conducted under a protocol approved by an Institutional Animal Care and Use Committee. Small variations in the concentrations of reagents, incubation times, etc. may occur and are expected to give similar results.

In most experiments, neurons were incubated in culture medium containing the FITC-labeled lectin BSI-B4 (IB4, 10 mg/ml) at 37° C. for 5 min before recording. The coverslip was washed with extracellular recording solution for 1 min before being placed in a recording chamber mounted on the stage of an inverted microscope equipped with fluorescence optics.

Electrophysiology: Electrophysiologic evaluation of neurons occurred within 4-48 h of plating. Whole cell patch-clamp recordings were obtained from dye-labeled DRG neurons. Recordings were obtained in an extracellular recording solution (pH 7.4, 295-320 mosM) consisting of (in mM) 140 NaCl, 3 KCl, 1 CaCl2, 1 MgCl2, 0.1 CdCl2, 10 HEPES, and 10 glucose. Patch-clamp electrodes were pulled from borosilicate glass and fire polished to 2-6 MOhm tip resistance. The internal pipette recording solution (pH 7.3, 290-300 mosM) consisted of (in mM) 140 CsCl, 10 NaCl, 1 EGTA, and 10 HEPES. Tetrodotoxin (TTX, 0.3 uM) was included in the extracellular solution to block TTX-sensitive sodium currents. Variations in the concentrations and types of reagents used for solutions may occur and are expected to give similar results.

Sodium currents were recorded from DRG neurons using standard electrophysiologic protocols. Neurons were typically voltage-clamped at −50 mV. Currents were recorded using a patch-clamp amplifier and digitized at 3-10 kHz for acquisition. Neuronal input resistance and membrane capacitance were determined from the amplitude and kinetics of the current response to a voltage pulse from a holding potential of −50 mV. Series resistance was compensated 75-95% for all recordings. Leak currents were cancelled online using a standard P/4 protocol. Depolarizing steps from −90, −70, or −50 mV to 0 mV were delivered every 5 or 30 sec during drug application to determine the effects of drugs on sodium currents. For all cell types, baseline responses were recorded for a minimum of 10 min to ensure that the kinetics of the response was stable. A wash out or recovery period usually followed the drug application period. Responses that exhibited long-lasting or irreversible changes in kinetics during the experiment were considered unstable and are not used for analysis. All data acquisition and analysis was performed using standard cell electrophysiology software. Variations in the details of electrophysiologic protocols may occur and are expected to give similar results.

For conditions where agents were either Ambroxol, Ralfinamide, Topiramate, or Sipatrigine, cells were constantly perfused with extracellular solution at a rate of 0.5-2 mi/mn in the recording chamber and agents were applied through the bath to individual cells. These agents were typically applied for 2-10 minutes, or until a steady-state drug effect was achieved. In these conditions, only TTX-R sodium currents were recorded from bladder afferent neurons since all recordings were performed in extracellular solution containing TTX (300 nM). Cumulative concentration-response curves were obtained from consecutive increases in drug concentration to each cell.

For the condition involving Lamotrigine, cells were constantly perfused with extracellular solution at a rate of approximately 1 ml/min in the recording chamber. Lamotrigine was applied through the bath to individual cells until a steady-state drug effect was achieved.

All data are expressed as mean±SEM.

Results and Conclusions

Bladder afferent neurons were identified as Di-I- or Fast Blue-positive neurons in in vitro DRG cultures.

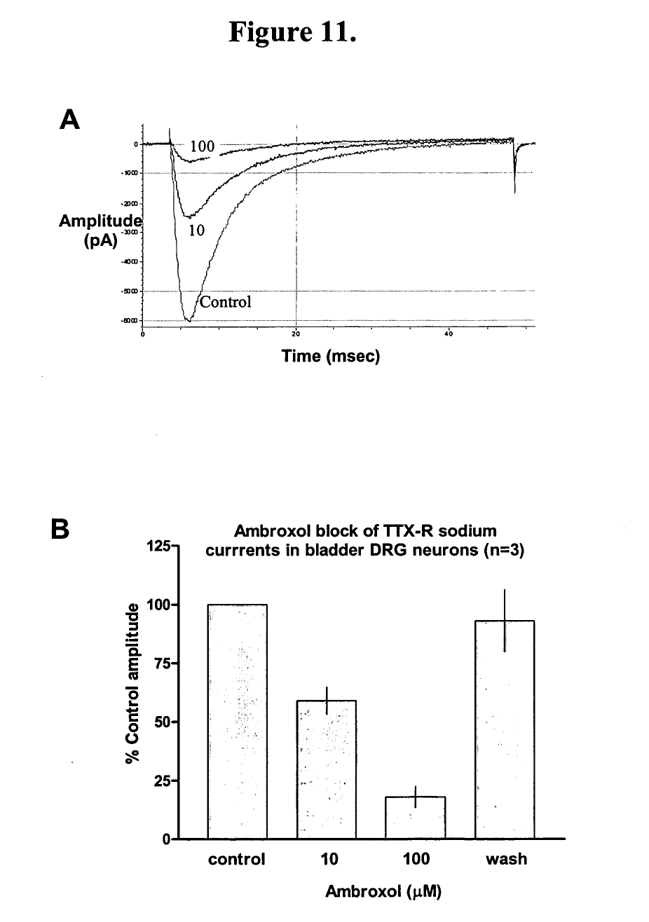

FIG. 11A shows a typical inward TTX-R sodium current recorded before (control) and during (10 and 100 μM) bath application of ambroxol. The kinetics of this and other responses recorded in similar bladder afferent neurons resembled the Nav.1.8 subtype of current. This is the “slow (Nav.1.8)” as opposed to the “persistent (Nav.1.9)” sodium current as described in Renganathan et al. (2002) J. Neurophysiol., 87:761-775. The neuron was voltage-clamped at −50 mV holding potential, and a 45 msec depolarizing pulse to 0 mV was delivered every 5 seconds. The control response was recorded prior to ambroxol application. A subsequent recording was made after a two minute application of 10 μM ambroxol, and another from the same neuron after an additional application of 100 μM ambroxol.

FIG. 11B shows that Ambroxol produced a concentration-dependent reversible block of TTX-R sodium currents in three bladder afferent neurons. The block occurred at an estimated IC50 concentration of 15 μM, consistent with selective block of TTX-R current by ambroxol (Weiser and Wilson (2002) Mol. Pharmacol. 62:433-438). Peak inward current amplitudes were measured when the responses had reached a steady-state in the presence of drug. Response amplitudes were normalized and mean +SEM are displayed. Ambroxol (2-3 minute application) produced a concentration-dependent reduction in current amplitude. The block was reversible, as response amplitudes recovered during a 2-5 minute wash period.

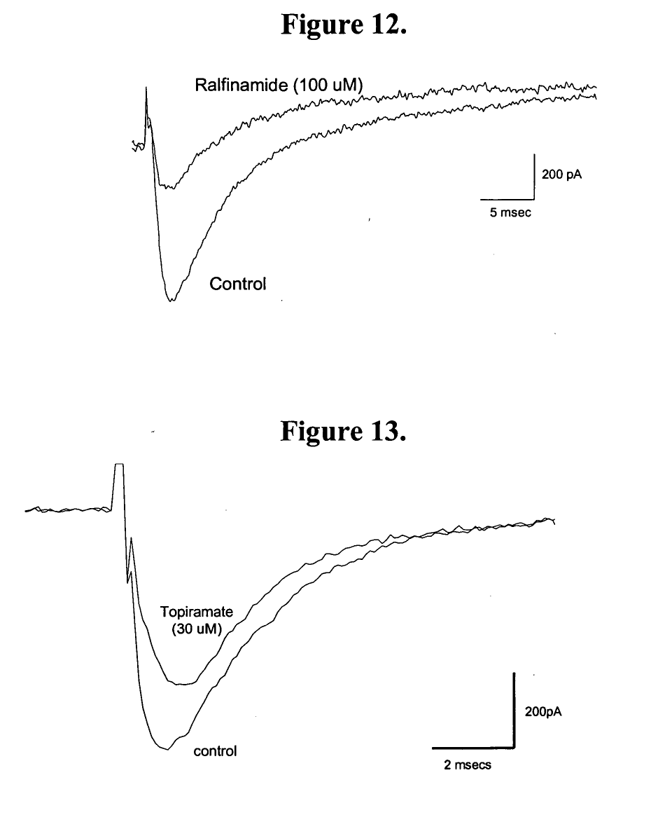

FIG. 12 shows a typical inward TTX-R sodium current recorded before (control) and during bath application of ralfinamide (100 μM). The neuron was voltage-clamped at −50 mV holding potential, and a 45 msec depolarizing pulse to 0 mV was delivered every 30 seconds. The control response was recorded prior to ralfinamide application. A subsequent recording was made after a 2 minute application of 100 μM ralfinamide. Ralfinamide blocked the current, indicative of its ability to decrease excitability of bladder afferent neurons. This effect was confirmed in three neurons where 100 μM ralfinamide blocked peak current to 36±6% of control.

FIG. 13 shows a typical inward TTX-R sodium current recorded before (control) and during bath application of topiramate (30 μM). The neuron was voltage-clamped at −70 mV holding potential, and a depolarizing pulse to +10 mV was delivered every 30 seconds. The control response was recorded prior to topiramate application. A subsequent recording was made after a 7 minute application of 30 μM topiramate. Topiramate blocked the current, indicative of its ability to decrease excitability of bladder afferent neurons.

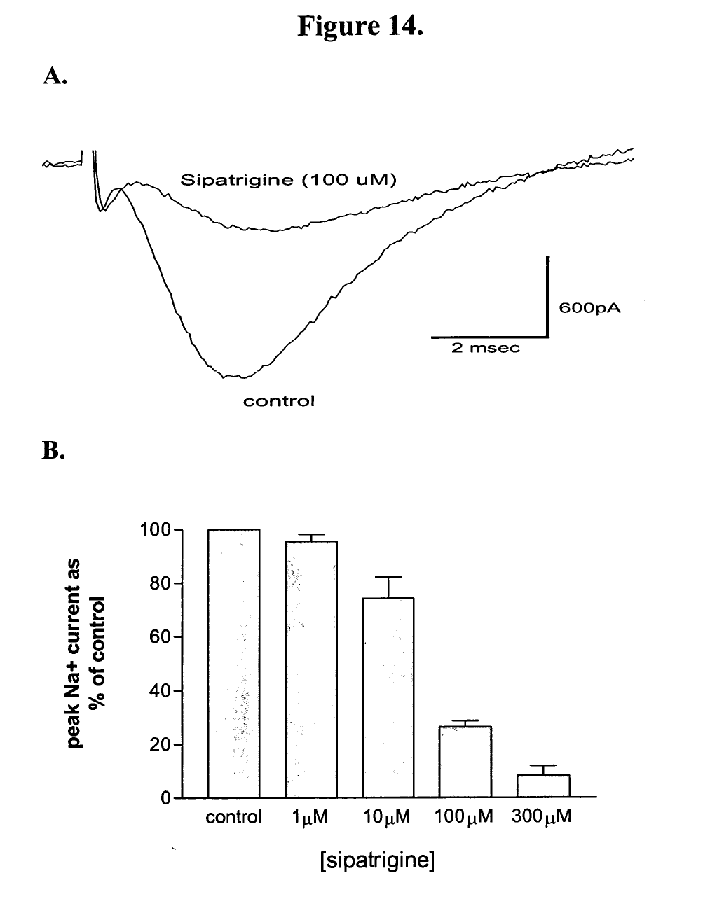

FIG. 14A shows a typical inward TTX-R sodium current recorded before (control) and during bath application of sipatrigine (100 μM). The neuron was voltage-clamped at −70 mV holding potential, and a depolarizing pulse to +10 mV was delivered every 10 seconds. The control response was recorded prior to sipatrigine application. A subsequent recording was made after a 6 minute application of 100 μM siptrigine. Sipatrigine blocked the current, indicative of its ability to decrease excitability of bladder afferent neurons.

FIG. 14B shows a summary concentration-response bar chart showing the combined effects of sipatrigine on 2-5 separate bladder afferent neurons. Peak inward current amplitudes were measured when the responses had reached a steady-state in the presence of drug. Response amplitudes were normalized and mean+SEM are displayed. Control responses were recorded before drug application. Sipatrigine produced a concentration-dependent reduction in current amplitude.

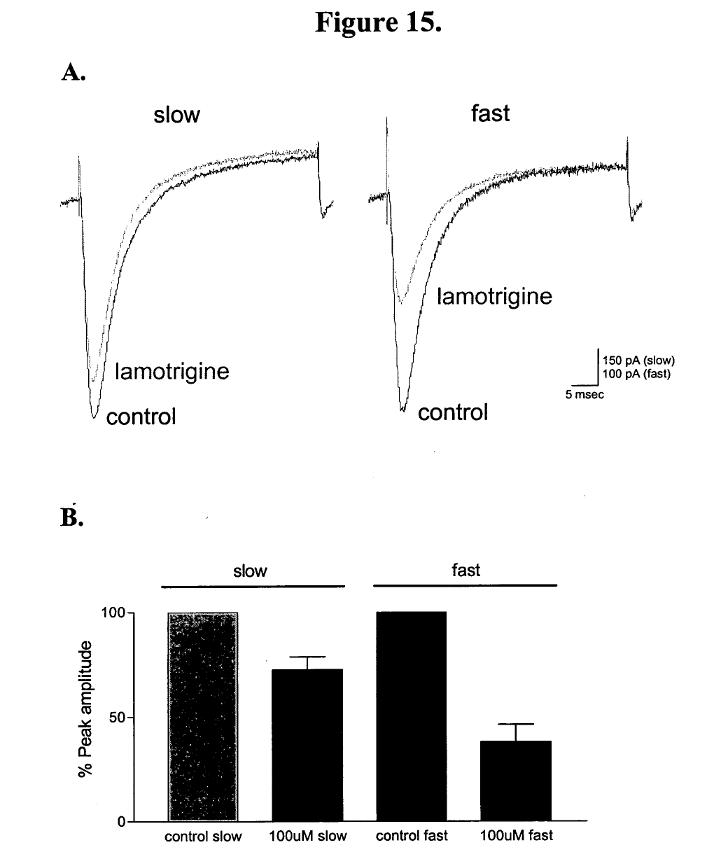

FIG. 15 demonstrates the use-dependent effects of lamotrigine (100 μM) on peak activity dependent sodium currents recorded in bladder DRG neurons. Slow activation of sodium currents consisted of step depolarizations from −50 to 0 mV delivered at a frequency of 0.2 Hz. Fast activation consisted of the same step depolarizations delivered at a frequency of 17 Hz. FIG. 1A shows a typical response to lamotrigine under both slow and fast stimulation protocols. Peak current amplitude was decreased to a greater extent under fast stimulation conditions, consistent with use-dependent modulation of bladder DRG sodium currents. FIG. 15B shows summary data obtained from three neurons. Data were obtained under control conditions and during application of 100 μM lamotrigine. The mean peak sodium current amplitude (expressed as % control amplitude) is decreased to a greater extent under fast stimulation conditions, consistent with modulation of bladder DRG sodium currents in a use-manner.

This example demonstrates the efficacy of sodium channel modulators in mammalian forms of painful and nonpainful lower urinary tract disorders including overactive bladder.

What is claimed is:

1. A method for treating a lower urinary tract disorder, which comprises administering to an individual in need thereof a therapeutically effective amount of an active agent wherein said agent is a sodium channel modulator or a pharmaceutically acceptable salt, ester, amide, prodrug, active metabolite or derivative thereof, and wherein said sodium channel modulator is not tolperisone or vinpocetine, and wherein said sodium channel modulator is not a semicarbazone or thiosemicarbazone when said lower urinary tract disorder is OAB Wet.

2. The method of claim 1, wherein the lower urinary tract disorder is selected from the group consisting of overactive bladder, prostatitis, prostadynia, interstitial cystitis, benign prostatic hyperplasia, and spastic bladder.

3. The method of claim 1, wherein the active agent is contained within a pharmaceutical formulation.

4. The method of claim 3, wherein the pharmaceutical formulation is a unit dosage formulation.

5. The method of claim 1, wherein the active agent is administered on an as-needed basis.

6. The method of claim 1, wherein the active agent is administered prior to commencement of an activity wherein suppression of the symptoms of a lower urinary tract disorder would be desirable.

7. The method of claim 6, wherein the active agent is administered from about 0 to about 3 hours prior to commencement of an activity wherein suppression of said symptoms would be desirable.

8. The method of claim 3, wherein the formulation is a controlled release dosage formulation.

9. The method of claim 8, wherein the formulation is a delayed release dosage formulation.

10. The method of claim 8, wherein the formulation is a sustained release dosage formulation.

11. The method of claim 9, wherein the formulation is a sustained release dosage formulation.

12. The method of claim 10, wherein the sustained release dosage formulation provides drug release over a time period of from about 6 hours to about 8 hours.

13. The method of claim 1, wherein the active agent is administered orally.

14. The method of claim 3, wherein the active agent is administered orally.

15. The method of claim 14, wherein the pharmaceutical formulation is selected from the group consisting of tablets, capsules, caplets, solutions, suspensions, syrups, granules, beads, powders and pellets.

16. The method of claim 1, wherein the active agent is administered transmucosally.

17. The method of claim 16, wherein the active agent is administered sublingually.

18. The method of claim 16, wherein the active agent is administered buccally.

19. The method of claim 16, wherein the active agent is administered intranasally.

20. The method of claim 16, wherein the active agent is administered transurethrally.

21. The method of claim 16, wherein the active agent is administered rectally.

22. The method of claim 16, wherein the active agent is administered by inhalation.

23. The method of claim 1, wherein the active agent is administered topically.

24. The method of claim 1, wherein the active agent is administered transdermally.

25. The method of claim 1, wherein the active agent is administered parenterally.

26. The method of claim 1, wherein the active agent is administered intrathecally.

27. The method of claim 1, wherein the lower urinary tract disorder is a painful lower urinary tract disorder.

28. The method of claim 1, wherein the lower urinary tract disorder is a non-painful lower urinary tract disorder.

29. The method of claim 28, wherein the non-painful lower urinary tract disorder is non-painful overactive bladder.

30. The method of claim 1, wherein the lower urinary tract disorder is selected from the group consisting of overactive bladder, prostatitis, prostadynia, interstitial cystitis, benign prostatic hyperplasia, and spastic bladder.

31. The method of claim 1, wherein said sodium channel modulator is:
a. a TTX-R sodium channel modulator, or a salt, enantiomer, analog, ester, amide, prodrug, active metabolite, and derivative thereof; or
b. an activity-dependent sodium channel modulator, or a salt, enantiomer, analog, ester, amide, prodrug, active metabolite, and derivative thereof.

32. The method of claim 31, wherein said TTX-R sodium channel modulator is:
a. a compound that interacts with Naᵥ1.8 channels, or a salt, enantiomer, analog, ester, amide, prodrug, active metabolite, and derivative thereof; or
b. a compound that interacts with Na_{v.}1.9 channels, or a salt, enantiomer, analog, ester, amide, prodrug, active metabolite, and derivative thereof.

33. The method of claim 1, wherein said sodium channel modulator is Ralfinamide or a salt, enantiomer, analog, ester, amide, prodrug, active metabolite, and derivative thereof.

34. The method of claim 1, wherein said sodium channel modulator is Ambroxol or a salt, enantiomer, analog, ester, amide, prodrug, active metabolite, and derivative thereof.

35. The method of claim 1, wherein said sodium channel modulator is Carbamazepine or a salt, enantiomer, analog, ester, amide, prodrug, active metabolite, and derivative thereof.

36. The method of claim 1, wherein said sodium channel modulator is Topiramate or a salt, enantiomer, analog, ester, amide, prodrug, active metabolite, and derivative thereof.

37. The method of claim 1, wherein said sodium channel modulator is Sipatrigine or a salt, enantiomer, analog, ester, amide, prodrug, active metabolite, and derivative thereof.

38. The method of claim 1, wherein said sodium channel modulator is Losigamone or a salt, enantiomer, analog, ester, amide, prodrug, active metabolite, and derivative thereof.

39. The method of claim 1, wherein said sodium channel modulator is Mexiletine or a salt, enantiomer, analog, ester, amide, prodrug, active metabolite, and derivative thereof.

40. The method of claim 1, wherein said sodium channel modulator is Lamotrigine or a salt, enantiomer, analog, ester, amide, prodrug, active metabolite, and derivative thereof.

41. The method of claim 2, wherein the pharmaceutical formulation further comprises an additional active agent.

42. The method of claim 41, wherein the additional active agent is selected from the group consisting of: an antispasmodic, a tricyclic antidepressant, duloxetine, venlafaxine, a monoamine reuptake inhibitor, a spasmolytic, an anticholinergic, gabapentin, pregabalin, a substituted aminomethyl-phenyl-cyclohexane derivative, a 5-HT₃ antagonist, a 5-HT₄ antagonist, a β3 adrenergic agonist, a neurokinin receptor antagonist, a bradykinin receptor antagonist, a nitric oxide donor, and derivatives thereof.

43. A method for treating overactive bladder, which comprises administering to an individual in need thereof a therapeutically effective amount of an active agent wherein said agent is a sodium channel modulator or a salt, enantiomer, analog, ester, amide, prodrug, active metabolite, and derivative thereof, and wherein said sodium channel modulator is not tolperisone or vinpocetine, and wherein said sodium channel modulator is not a and wherein said sodium channel modulator is not tolperisone or vinpocetine, and wherein said sodium channel modulator is not a semicarbazone or thiosemicarbazone when said lower urinary tract disorder is OAB Wet.

44. A pharmaceutical formulation for treating overactive bladder and adapted for transmucosal drug administration, comprising a therapeutically effective amount of a sodium channel modulator, or a pharmaceutically acceptable salt, ester, amide, prodrug, or active metabolite thereof, and a carrier suitable for transmucosal drug delivery buccally, sublingually, intranasally, rectally, or by inhalation, and wherein said sodium channel modulator is not tolperisone or vinpocetine, and wherein said sodium channel modulator is not a semicarbazone or thiosemicarbazone when said lower urinary tract disorder is OAB Wet.

45. A packaged kit for a patient to use in the treatment of overactive bladder, comprising: a pharmaceutical formulation of a sodium channel modulator; a container housing the pharmaceutical formulation during storage and prior to administration; and instructions for carrying out drug administration in a manner effective to treat overactive bladder, and wherein said sodium channel modulator is not tolperisone or vinpocetine, and wherein said sodium channel modulator is not a semicarbazone or thiosemicarbazone when said lower urinary tract disorder is OAB Wet.
